(19) United States
(12) Patent Application Publication
(10) Pub. No.: US 2021/0187084 A1
(43) Pub. Date: Jun. 24, 2021

(54) INJECTION TECHNIQUES FOR THE TREATMENT OF CELLULITE

(71) Applicant: ENDO GLOBAL AESTHETICS LIMITED, Dublin (IE)

(72) Inventors: Saji VIJAYAN, Chester Springs, PA (US); Matthew W. DAVIS, Malvern, PA (US); Michael MCLANE, Lansdale, PA (US); George OMBURO, Jersey City, NJ (US); Todd KIRBY, Pottstown, PA (US)

(21) Appl. No.: 17/259,784

(22) PCT Filed: Jul. 12, 2019
(86) PCT No.: PCT/IB2019/000777

§ 371 Date: Jan. 12, 2021

Related U.S. Application Data
(60) Provisional application No. 62/697,376, filed on Jul. 12, 2018.
(60) Provisional application No. 62/733,046, filed on Sep. 18, 2018.

(30) Foreign Application Priority Data
Jul. 11, 2019 (IB) PCT/IB2019/000767

(51) Int. Cl. A61K 38/48; A61K 9/00; A61P 17/00

(52) U.S. Cl. CPC A61K 38/4886; A61P 17/00; A61K 9/0019

(57) ABSTRACT

The present disclosure relates to a method of treating cellulite on a thigh or buttock in a human subject by administering an effective amount of collagenase, and then assessing the reduction in the severity of cellulite by one or more scales.

### RELATED APPLICATIONS

This application claims priority to International Application No. PCT/US19/41494 filed on Jul. 11, 2019, U.S. Provisional Application Ser. No. 62/697,376 filed on Jul. 12, 2018 and U.S. Provisional Application Ser. No. 62/733,046 filed on Sep. 18, 2018, which are incorporated herein by reference in their entirety to the full extent permitted by law.

### TECHNICAL FIELD

The present invention relates to the field of assessing and treating cellulite.

### BACKGROUND

Cellulite (also known as edematous fibrosclerotic panniculopathy (EFP)), is an aesthetic condition that can be understood as an imbalance between the structural characteristics and biomechanical properties (i.e., the delicate containment and extrusion forces) at the subdermal junction (Rudolph et al, “Structural Gender Dimorphism and the Biomechanics of the Gluteal Subcutaneous Tissue: Implications for the Pathophysiology of Cellulite,” Plast. Reconstr. Surg. 2019; 143(4):1077-1086). Accordingly, the goals of cellulite treatment are to strengthen the subdermal interface and/or to release the fibrous septae via various types of subcision (Rudolph et al., supra). The fibrous septae has been recognized as a contributory underlying cause of cellulite and as a target of treatment for cellulite by anatomical and image analyses studies (Hexsel et al, “Side-by-side comparison of areas with and without cellulite depressions using magnetic resonance imaging,” Dermatol Surg. 2009; 35(10):1471-1477; Hexsel et al. “Magnetic Resonance Imaging of Cellulite Depressed Lesions Successfully Treated by Subcision,” Dermatol Surg. 2016; 42(5):693-696; Mirrashed F, Sharp J C, Krause V, Morgan J, Tomanek B. “Pilot study of dermal subcutaneous fat structures by MRI in individuals who differ in gender, BMI, and cellulite grading,” Skin Res Technol. 2004; 10(3):161-168; Nürnberger and Müller, “So-called cellulite: an invented disease,” J Dermatol Surg Oncol. 1978; 4(3):221-229; Piérard et al, “Cellulite: from standing fat herniation to hypodermal stretch marks,” Am J Dermatopath. 2000; 22(1):34-37; Querleux et al, “Anatomy and physiology of subcutaneous adipose tissue by in vivo magnetic resonance imaging and spectroscopy: relationships with sex and presence of cellulite,” Skin Res Technol. 2002; 8(2):118-124). To effectively treat cellulite, a therapeutic approach is needed to lyse or otherwise disrupt the dermal septa, which are composed of collagen (FIG. 1) and cause the skin dimpling that is bothersome to many women.

There are therapies that have been utilized in an attempt to treat cellulite; however, there are no approved pharmacologic treatments. Despite multiple therapeutic modalities, there is little scientific evidence that any of the current non-pharmacologic treatments are beneficial. In fact, much of the evidence is anecdotal, subjective, or based only on patient self-assessment. Some of the historical treatments for EFP have included weight loss, topical agents, massage, liposuction, mesotherapy, radiofrequency, subcision, powered subcision, and laser therapies. Many of these treatments have undesirable side effects (Avram M M, “Cellulite: a review of its physiology and treatment,” J Cosmet Laser Ther. 2004; 6(4):181-185; Collis et al, “Cellulite treatment: a myth or reality: a prospective randomized, controlled trial of two therapies, endermologie and aminophylline cream,” Plast Reconstr Surg. 1999; 104(4):1110-1114; Khan M H, Victor F, Rao B, Sadick N S. “Treatment of cellulite: Part I. Pathophysiology.” J Am Acad Dermatol. 2010; 62(3):361-370; Hexsel D M, Mazzuco R. “Subcision: a treatment for cellulite.” Int J Dermatol. 2000; 39(7):539-544; Boyce et al, “Clinical evaluation of a device for the treatment of cellulite: Triactive.” Am J Cosmet Surg. 2005; 22:233-237; DiBernardo B E. “Treatment of cellulite using a 1440-nm pulsed laser with one-year follow-up.” Aesthet Surg J. 2011; 31(3):328-341). As such, many physicians are of the view that improvements for aesthetic conditions are not easily obtained. Thus, there remains an unmet need for safe and effective nonsurgical therapies to improve the aesthetic outcome in women with cellulite.

### SUMMARY

The present disclosure satisfies the above need and relates to methods of treating cellulite in human patients by the subcutaneous injection of a therapeutically effective amount of collagenase (as defined in the Detailed Description). Such methods relate to the pretreatment assessment of a patient's severity of cellulite using various scales and assessment techniques to establish the patient's baseline of cellulite severity. This is then followed by the treatment of the cellulite by the subcutaneous injection of collagenase. The dosing and administration of the collagenase may vary, and the collagenase may be in the form of a pharmaceutical composition comprising the collagenase and one or more pharmaceutically acceptable excipients. Such excipients may include sterile water for injection, pH adjusting agents and stabilizers. Post-treatment assessments are performed to confirm the efficacy of the treatment compared to baseline. The methods of treatments of the present disclosure result in significant reductions in the appearance of cellulite.

As explained in the Detailed Description, there are four phases of treatment, although they are optional and the order is not intended to be strictly limiting.

1. In a first phase, the clinician performs a selection of cellulite dimples to be treated. Next, before injection, an assessment is performed, e.g., the clinician and/or patient independently assess the pretreatment severity of cellulite using one or more of the following scales or other assessment methods (as defined in the Detailed Description):
Hexsel Cellulite Severity Scale (Hexsel CSS)
Hexsel Depression Depth Score
Likert Scale
Dimple Analysis
Clinician Reported Photonumeric Cellulite Severity Scale (CR-PCSS)
Patient Reported Photonumeric Cellulite Severity Scale (PR-PCSS)
Investigator Global Aesthetic Improvement Scale (I-GAIS)
Subject Global Aesthetic Improvement Scale (S-GAIS)
Patient Reported Cellulite Impact Scale (PR-CIS)
PR-CIS Abbreviated
Subject Self-Rating Scale (SSRS)
Subject Satisfaction with Cellulite Treatment (SSCT)
Clinician assessment of cellulite severity (photography or other imagery)
Body-Q
Fitzpatrick scale
Thigh Cellulite Efficacy Scale (PR-TCES; CR-TCES)
Any validated photonumeric or other scale used by clinicians and/or patients to assess cellulite severity, improvement, and/or patient satisfaction (e.g., Hexsel-Merz Scale (poster publication at American Academy of Dermatology meeting 2019).
   Further, the pretreatment assessment by clinicians and patients may be performed by analyzing a series of 3 to 15 photographs, illustrations, drawings, computer images, 3-D models, MRI images, thermograms, ultrasonograms, patient verbal feedback or the like each having a different cellulite severity rating or level.
   2. In a second phase of treatment, dimples to be treated are marked by the clinician with a dot or other marking (FIG. 6). It is typically placed at the nadir of the dimple, if a nadir is present. More photographs may be taken and other assessments performed.
   3. In a third phase of treatment, a therapeutically effective amount of collagenase is injected subcutaneously into the dimple(s) in a single dose or divided doses at one or more treatment areas (as defined in the Detailed Description). The doses and injection techniques vary. For example, the method may comprise deep and shallow injections according to the following procedure (illustrated in FIG. 10 (Treatment IV)):
With the needle positioned at an angle of approximately 30° to the skin surface at the injection site and directed towards subject's head, the needle is pushed all the way in (1 inch) and 0.3 mL of collagenase composition is injected by gently pushing the syringe plunger.
About half the length of the needle (½ inch) is gently withdrawn while maintaining the 30° angle of the needle to skin surface, and the needle is repositioned towards one side of the subject (Position B). About 0.3 mL collagenase composition is injected by gently pushing the syringe plunger.
The 30° angle of the needle is maintained to the skin surface, and the needle is repositioned to midway between Position A and B (Position C, towards subject's shoulder) and about 0.3 mL of collagenase composition is injected by gently pushing the syringe plunger.
The 30° angle of the needle is maintained to the skin surface, and the needle is repositioned exactly opposite to Position B (Position D), and about 0.3 mL of collagenase composition is injected by gently pushing the syringe plunger.
The 30° angle of the needle is maintained to the skin surface, and the needle is repositioned to midway between Position A and D (Position E, towards subject's other shoulder) and about 0.3 mL of collagenase composition is injected by gently pushing the syringe plunger.
A 3 mL syringe with 1.5 mL of collagenase composition is sufficient for one injection site.
Twelve (12) 3 mL syringes are used in each treatment area (each buttock or each thigh) to administer a total of 12 injections of 1.5 mL (total of 18.0 mL, 5 aliquots 0.3 mL each) at 12 injection sites.
A total of 24 injections is administered across the 2 treatment areas (2 buttocks or 2 thighs) at each treatment visit.

In another example, the needle may be injected at three angles into a dimple: At about 90 degrees to the skin surface, at about 45 degrees to the skin surface and long axis of the dimple, and at about 135 degrees to the skin surface and long axis of the dimple. In one non-limiting example, the three injections above are delivered directly to the dimple as three 0.1 mL aliquots utilizing a 1 mL syringe with 0.1 mL gradients and 30-guage ½ inch needle. Other techniques are explained in the Detailed Description.

4. In a fourth phase of treatment, post-injection assessments are performed using the above-mentioned scales and other assessment methods (e.g., bruising analysis). Efficacy of a particular collagenase treatment may be based on a single clinician rating or patient rating, or based on a composite endpoint comprising the clinician rating and the patient rating where improvement is shown in both scales for the same subject, i.e., a pre-specified level of improvement is demonstrated in both the clinician and patient scales.

The collagenase is injected in an amount of about 0.01 mg to about 20 mg in a single dose or divided doses, and has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden
   As used herein, the relevant kinetic parameters may be measured using the cuvette assays or microplate assays (e.g., the SRC cuvette assay, the SRC microplate assay, the GPA cuvette assay, and the GPA microplate assay) as described herein.

In some embodiments, the collagenase present in the composition comprises collagenase I and collagenase II in a ratio of approximately 1:1. Other ratios of collagenase I and collagenase II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of collagenase I and collagenase II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In another embodiment, the collagenase composition comprises CCH (as defined in the Detailed Description) having an AUX I and AUX II ratio of approximately 1:1. Other ratios of AUX I and AUX II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of AUX I and AUX II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In other examples, the collagenase composition may be a liquid or is reconstituted from a lyophilized solid form with a diluent. The dose of the mixture is measured by the amount of collagenase present without regard to diluent, and may comprise about 0.1 mg to about 20 mg in one or more injections. In another embodiment, the dose administered is about 0.06 mg, 0.48 mg, 0.84 mg, 1.68 mg, 2.52 mg, 3.36 mg, 4.2 mg, 5.04 mg, 5.88 mg, 6.72 mg, 7.56 mg, or 8.4 mg in one or more injections. For instance, about 0.06 mg, 0.48 mg, 0.84 mg, or 1.68 mg is administered in about 12 divided injections. The volume of collagenase composition injected may range from 0.01 mL to 3 mL per injection, or total about 0.2 mL to 150 mL per treatment visit (as defined in the Detailed Description). In a specific embodiment, the above doses are to a collagenase composition comprising CCH. In another embodiment, the above doses are to a collagenase composition having one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM^{31 1}sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another embodiment, about 0.84 mg of CCH is injected in about 12 equally divided injections per treatment area (about 0.07 mg×12 injections=about 0.84 mg). In some cases, such treatment with 0.84 mg occurs in one treatment visit, or every 10-40 days for 2, 3, 4 or 5 treatment visits. In other cases, more than one treatment area is injected with 0.84 mg every 10-40 days for 2, 3, 4 or 5 treatment visits. Such injections may be administered in more than 5 treatment visits.

Further, as described in the Detailed Description, the collagenase injections are effective in treating cellulite. For example, significant improvements in the appearance of cellulite are demonstrated by Hexsel Depression Depth Scores, Likert scale scores and by dimple analysis.

Additional embodiments of the present composition, scales, methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1 is a cross-sectional illustration of skin and subdermal tissue depicting the collagen septae.

FIG. 2 is an amino acid sequence listing for AUX-I (Seq. ID No. 5).

FIG. 3 is an amino acid sequence listing for AUX-II (Seq. No. ID 6).

FIG. 4 illustrates the Hexsel cellulite severity scale (CSS) (B) depth of depressions.

FIG. 5 illustrates the PR- and CR-Thigh Cellulite Efficacy Scale.

FIG. 6 illustrates an example of subject dimple and injection site markings on the buttock.

FIG. 7 depicts the injection technique used in Treatment I (shallow injection, 3 aliquots).

FIG. 8 depicts the injection technique used in Treatment II (shallow injection, 1 aliquot).

FIG. 9 depicts the injection technique used in Treatment III (deep injection, 1 aliquot).

FIG. 10 depicts the injection technique used in Treatment IV (deep and shallow injections, 5 aliquots).

FIG. 11 depicts the injection technique used in Treatment V (shallow injections, 4 aliquots).

FIG. 12 is an illustration of an injection technique useful in administering collagenase or placebo to a cellulite dimple.

FIG. 13 is a 3-D registration to grid (Day 1 Pre-Marking).

FIG. 14 is a color-by-distance map for image registration.

FIG. 15 is a primary dimple of the area of interest.

FIG. 16 is a transposing the primary dimple of the area of interest.

FIG. 17 depicts the outline of the normal tissue and bruised tissue at Days 4, 8, and 15 after injection in the left buttock of a subject.

FIG. 18(A) depicts the outline of the normal tissue and bruised tissue at Days 4, 8, and 15 after injection in the left buttock and provides L*, a*, and b* color measurements in those tissues.

FIG. 18(B) depicts the outline of the normal tissue and bruised tissue at Days 4, 8, and 15 after injection in the left buttock. Average color and AFs for the normal and bruised tissues are calculated based on the L*a*b* color values.

FIGS. 19(A)-19(C) depicts a dimple analysis. FIG. 19(A) illustrates the observed and change from Day 1 pre-marking image in dimple analysis parameters. FIG. 19(B) illustrates the maximum length and maximum width of the dimple. FIG. 19(C) illustrates the surface area and volume between the dimple base and interpolated surface.

FIG. 20 depicts the study flow chart of the phase 2b, randomized, double-blind, placebo-controlled study of CCH in the treatment of Edematous Fibrosclerotic Panniculopathy in thighs.

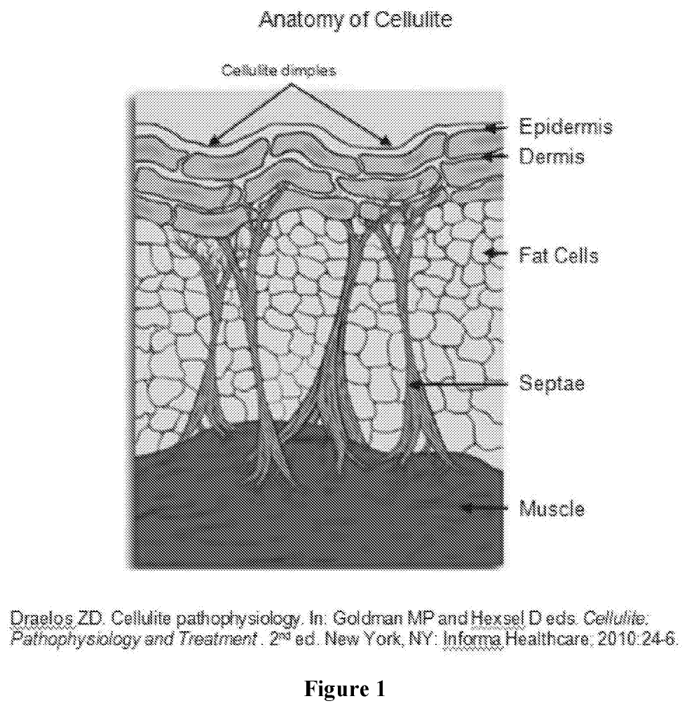

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

### A. Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

“Affected area” or “treatment area” as used herein means an area of cellulite on a human patient that is to be treated or has been treated with collagenase (defined below). This may include a quadrant (i.e., left buttock, right buttock, left posterolateral thigh, right posterolateral thigh). Affected area or treatment area is not limited to buttocks or thighs. Rather, any area of the body with cellulite can be treated as a treatment area.

“Adverse Events” or “AE” as used herein means any unfavorable or unintended change in body structure (signs), body function (symptoms), laboratory result (e.g., chemistry, ECG, X-ray, etc.), or worsening of a preexisting condition associated temporally with the use of the study medication whether or not considered related to the study medication.

“Body-Q” as used herein is a patient-reported outcome instrument that is commercially available under license from Memorial Sloan Kettering Cancer Center. It is based on patient perceptions of body contouring and/or weight loss. It measures 3 domains: appearance, health-related quality of life (HRQL), and patient experience of healthcare through 18 independently functioning scales. The patient-reported outcome instrument is described in BODY-Q: User's Manual BODY-Q: User's Manual, Version 1.0, July 2015, Memorial Sloan Kettering Cancer Center, McMaster University and Stefan Cano. The BODY-Q includes a scale to measure cellulite. See https://www.mskcc.org/sites/default/files/node/174457/documents/body-q-users-guide.pdf (accessed Jul. 3, 2019). For cellulite, there are 16 scaled items having response options ranging from “not at all” to “extremely bothered” over the timeframe of the past week and assuming a Flesch-Kincaid grade reading level. The patient is asked: “With your cellulite in mind, in the past week, how much have you been bothered by:” [16 questions follow where the patient ranks the response as 1-extremely bothered; 2-moderately bothered; 3-a little bothered; 4-not bothered at all]. The score ranges from 16 (extremely bothered) to 64 (not at all).

Conventionally, clinical examination of bruising comprises a visual examination of the bruised and surrounding areas in conjunction with an evaluation of the subject's medical, surgical, and concomitant medication histories. The results of this interpretation are subjective and affected by several unrelated factors, including viewing geometry, ambient lighting, color of unexposed surrounding skin, and the experience and visual acuity of the observer. “Bruising Analysis” as used herein means the detection of visible change in skin color as evaluated from the images of the collagenase-treated areas in a subject using the objective image capture and tracking methodologies disclosed in U.S. Patent Publication No. 2019/0035080 applied uniformly to all subject images. This objective analysis has the potential to aid or even replace visual and clinical examination of the bruising by the health care provider by providing the ability to quantify, differentiate, and assess the bruising both intra-subject (within the same subject at different times points) and inter-subject (between different subjects) levels. This analysis utilizes the L*a*b* color space defined by the Commission Internationale de l'Eclairage (CIE) modeled after a color-opponent theory stating that two colors cannot be red and green at the same time or yellow and blue at the same time. As shown below, L* indicates lightness/darkness, a* is the red/green coordinate, and b* is the yellow/blue coordinate. Deltas for L* (ΔL*), a* (Δa*) and b* (Δb*) may be positive (+) or negative (−). The total difference, Delta E (AE*), however, is always positive.
ΔL* (L* sample minus L* standard)=difference in lightness and darkness (+=lighter, −=darker); a low number (0-50) indicates dark and a high number (51-100) indicates light
Δa* (a* sample minus a* standard)=difference in red and green (+=redder, −=greener)
Δb* (b* sample minus b* standard)=difference in yellow and blue (+=yellower, −=bluer)

All three values are required to completely describe an object's color (in this case the bruising captured in the treated area image of the subject). The objective methodology for the image analysis of collagenase-treated area (pre- and post-treatment images at protocol specified time points) with data outputs in L*a*b* values allow for quick, easy, accurate, repeatable, and unbiased quantification of skin color and any change therein. This methodology rules out the inherent variability associated with the conventional subjective visual estimation of the images. A ΔE is calculated as follows:

ΔE(Color difference between-Bruised vs. Normal)=SQRT[(L*_{B}−L*_{N})+(A*_{B}−A*_{N})+(B*_{B}−B*_{N})²], where

### L*_{B}=Bruised Tissue L*

### L*_{N}=Normal Tissue L*

### A*_{B}=Bruised Tissue A*

### A*_{N}=Normal Tissue A*

### B*_{B}=Bruised Tissue B*

### B*_{N}=Normal Tissue B*

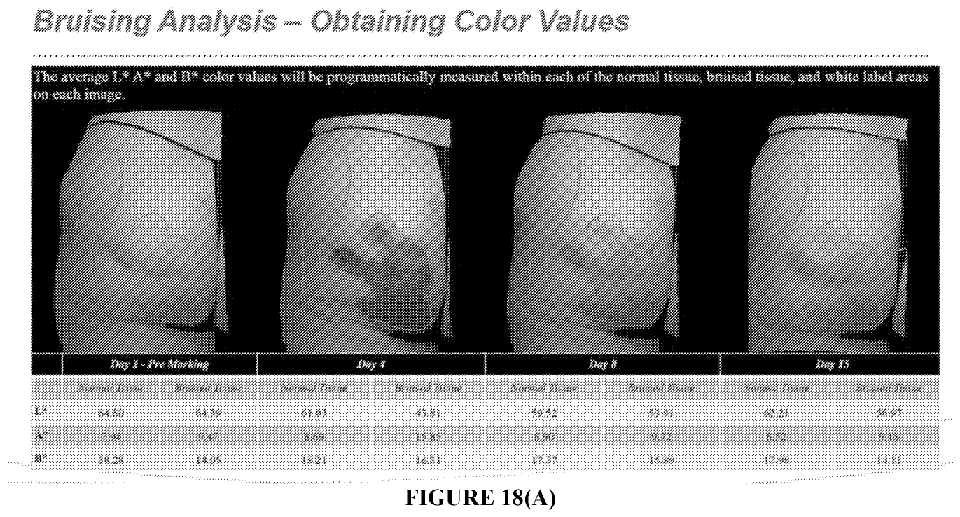

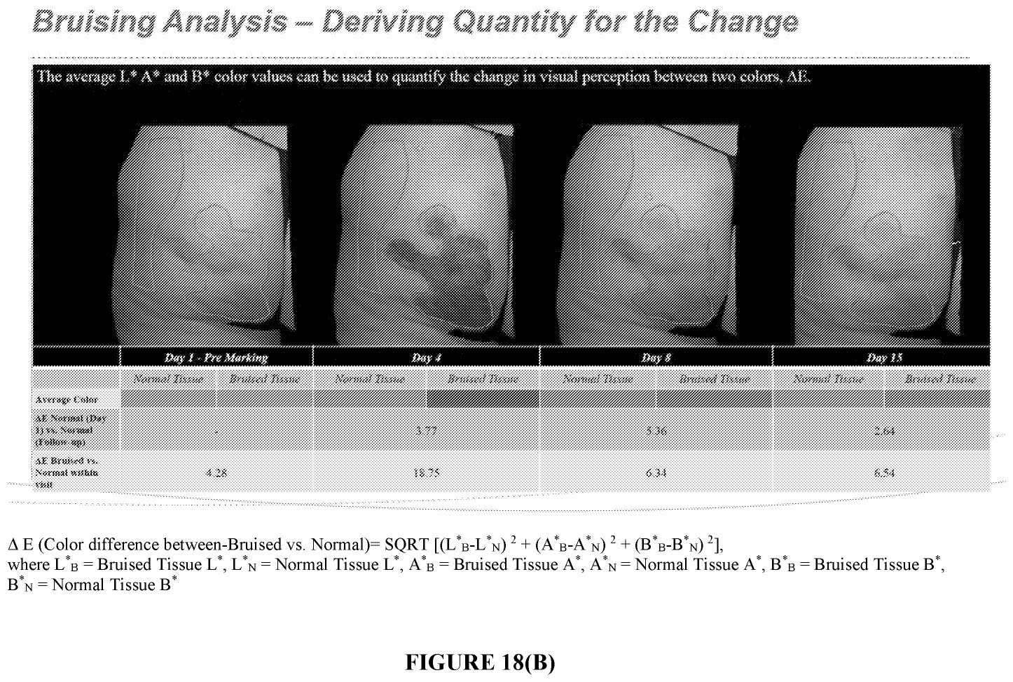

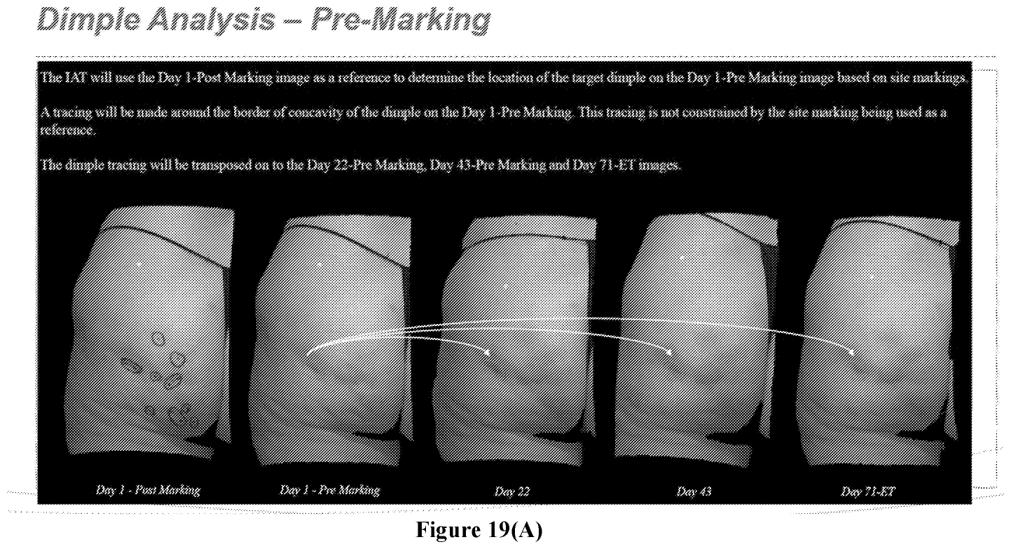

FIG. 18(B) illustrates a bruise analysis of a treatment area.

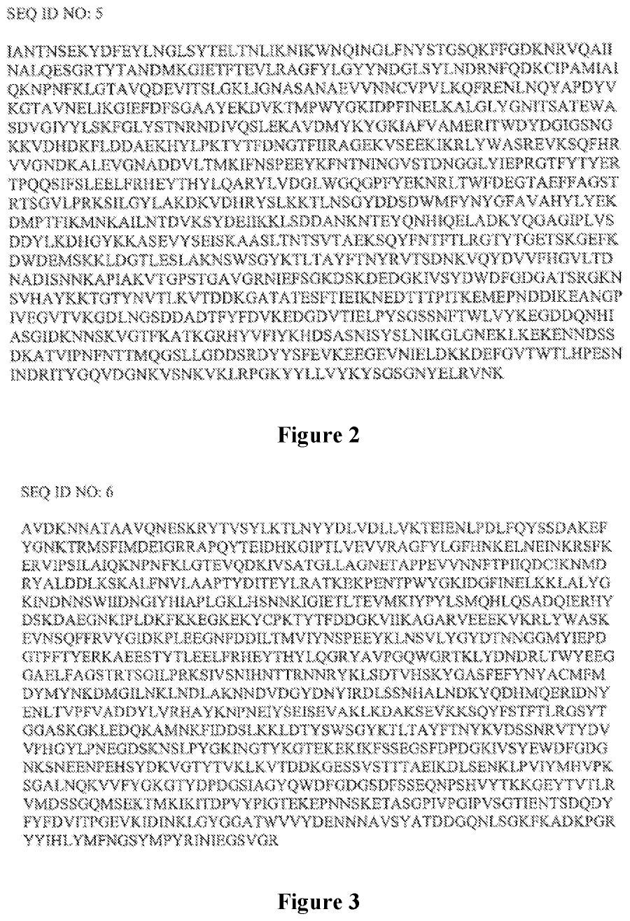

“CCH” as used herein means the AUX-I (Seq. ID No. 5 (FIG. 2)) and AUX-II (Seq. No. ID 6 (FIG. 3)) mixture of collagenases in an approximate 1:1 ratio obtained by the fermentation of Clostridium histolyticum (also known as Hathewaya histolytica). CCH is available commercially as a lyophilized powder under the trademark XIAFLEX®, which comprises the AUX-I and AUX-II mixture with particular excipients, although CCH may be used with other suitable excipients.

“Clinician Reported Photonumeric Cellulite Severity Scale (CR-PCSS)” as used herein are the photonumeric scales described in PCT Patent Application PCT/US2018/020551 (published as WO2018/160905 on Sep. 7, 2018) used by physicians/clinicians and designed to assess the severity of cellulite into 5 levels.

Except as otherwise provided herein, “collagenase” means any of the following: (a) collagenase (including mutants) having activity as defined by EC 3.4.24.3 (https://www.brenda-enzymes.org/enzyme.php?ecno=3.4.24.3 (accessed Jul. 3, 2019); (b) collagenase produced by fermentation of Clostridium histolyticum (also known as Hathewaya histolytica); (c) CCH (as defined above); (d) collagenase having at least 50% sequence alignment with AUX-I as determined by BLAST; (e) collagenase having at least 50% sequence alignment with AUX-II as determined by BLAST; (f) collagenase produced by fermentation of other source organisms (i.e., non-Clostridium histolyticum), e.g., mammalian, crustacean, fungal, bacterial or microbial collagenase; (g) collagenase obtained by recombinant techniques; (h) collagenase with a molecular mass from about 65 kDa to about 130 kDa; (i) collagenase designated as class I or class II (also referred to as collagenase I (or 1), collagenase II (or 2), Type I collagenase, Type 2 collagenase); (j) mixtures of collagenases I and II; (k) collagenase from strain JCM 1403 (ATCC 19401) or derivatives thereof; (1) collagenase from strain ATCC 21000 or derivatives thereof; (m) collagenase from ATCC 69334 or derivatives thereof; (n) collagenase from C. perfringens; (o) collagenase from Vibrio alginolyticus; (p) collagenase from Streptomyces; (q) collagenase from Pseudomonas; (r) collagenase from Achromobacter iophagus (s) collagenase described by Worthington Biochemical Corp. (www.Worthington-biochem.com; “Product Highlights”); (t) collagenase described by Sigma-Aldrich (www.sigma-aldrich.com); (u) collagenase having one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM^{31 1}sec⁻¹ of about 5,140 to 508,814 (SRC assay), or about 60 to 5,934 (GPA assay);
(v) collagenase described by Nordmark Arzneimittel GmbH & Co. KG; (w) collagenase from strain 004; or (x) equivalents or mixtures of any of the foregoing. Non-limiting examples of collagenases that may be used in the disclosure herein are described in U.S. Pat. Nos. 7,811,560; 9,757,435; 9,744,138; and WO2012/125948.

“Dimple analysis” as used herein means an analysis of one or more selected dimples wherein parameters, such as dimple volume, length, width and surface area are measured. Measurements may be performed by various known methods such as those described in Eckhouse et al. WO 2018/116304 and WO 2018/116305, and from Cherry Imaging (www.cherryimaging.com) and Canfield Scientific, Inc. See also Salameh et al., “Novel Stereoscopic Optical System for Objectively Measuring Above-Surface Scar Volume First—Time Quantification of Responses to Various Treatment Modalities,” Dermatol. Surg. 00:1-7 (2017); and U.S. Pat. No. 9,996,923. Such measurements of volume, length, width and surface area may be calculated using digital 3-D greyscale images (with X and Y axis rotation feature) and digital 3-D textured and lit images (with X and Y rotation feature) together with a computer program that analyzes such images. As an example, for a buttock treatment area, images may be taken of the left treated buttock and/or right treated buttock for each patient before and after treatment. For a thigh treatment area, images may be taken of each of the thigh treated areas at 0 degrees, 45 degrees and 90 degrees before and after treatment. For a thigh treatment area, images taken using the method by Canfield Scientific may be taken of each of the thigh treated areas at 0 degrees, 45 degrees and 90 degrees before and after treatment.

“Durability” as used herein means the period of time in which there is a persistence of a treatment effect. This period of time can range from about 3 months to about 20 years, or about 1 to 19 years, or about 2 to 18 years, or about 3 to 17 years, or about 4 to 16 years, or about 5 to 15 years, or about 6 to 14 years, or about 7 to 13 years, or about 8 to 12 years, or about 9 to 11 years. The period may be for about 6 months, about 1 year, about 2 years, about 3 years, about 4 years, about 5 years, about 10 years, about 15 years, or about 20 years.

“Fitzpatrick scale” as used herein means a scale is used to assess a subject's skin type as shown in Table 1.

TABLE 1
Fitzpatrick Scale

| | | |
|---|---|---|
| I | Pale white skin, blue/hazel eyes, | Always burns, does not tan |
| | blond/red hair | |
| II | Fair skin, blue eyes | Burns easily, tans poorly |
| III | Darker white skin | Tans after initial burn |
| IV | Light brown skin | Burns minimally, tans easily |
| V | Brown skin | Rarely burns, tans darkly easily |
| VI | Dark brown or black skin | Never burns, always tans darkly |

“Hexsel Cellulite Severity Scale” or “Hexsel CSS” or “Cellulite Severity Scale (CSS)” as used herein means the following photonumeric scale that evaluates 5 key morphologic features of cellulite (Table 2):

TABLE 2
Hexsel Cellulite Severity Scale

| | | |
|---|---|---|
| A | Number of evident depressions | 0 = no depressions |
| | | 1 = small amount: 1-4 depressions are visible |
| | | 2 = Moderate amount: 5-9 depressions |
| | | 3 = large amount: 10 or more depressions |
| B | Depth of depressions | 0 = no depressions |
| | | 1 = superficial depressions |
| | | 2 = medium depth depressions |
| | | 3 = deep depressions |
| C | Morphological appearance | 0 = no raised areas |
| | of skin surface alterations | 1 = orange peel appearance |
| | | 2 = cottage cheese appearance |
| | | 3 = mattress appearance |
| D | Grade of laxity, flaccidity, | 0 = absence of laxity, flaccidity, or sagging skin |
| | or sagging skin | 1 = slight draped appearance |
| | | 2 = moderate draped appearance |
| | | 3 = severe draped appearance |
| E | Classification scale by | Stage 0 = No dimpling when the subject is standing and lying. |
| | Nürnberger and | The pinch test reveals “folds and furrows”, but there is no |
| | Müllera | mattress-like appearance. |
| | | Stage 1 = No dimpling while the subject is standing or lying, |
| | | but the pinch test reveals the mattress-like appearance. |
| | | Stage 2 = Dimpling appears spontaneously when standing and not |
| | | lying down. |
| | | Stage 3 = Dimpling is spontaneously positive standing and |
| | | lying down. |

The sum of points results in the following classification.

| Points | Classification of Cellulite |
|---|---|
| 1-5 | Mild |
| 6-10 | Moderate |
| 11-15 | Severe |

Hexsel et al., J. Eur. Acad., Dermatol. Venereol. 2009; 23(5): 523-528. a. Nürnberger and Müller, J. Dermatol. Surg. Oncol. 1978; 4(3): 221-229. Subjects were evaluated in the standing position with relaxed gluteus muscles. However, if the subject had no evident depressions, they were asked to contract their gluteus muscles or the pinch test was applied (by pinching the skin between the thumb and index finger) in order to differentiate between scores/grades of zero or 1.

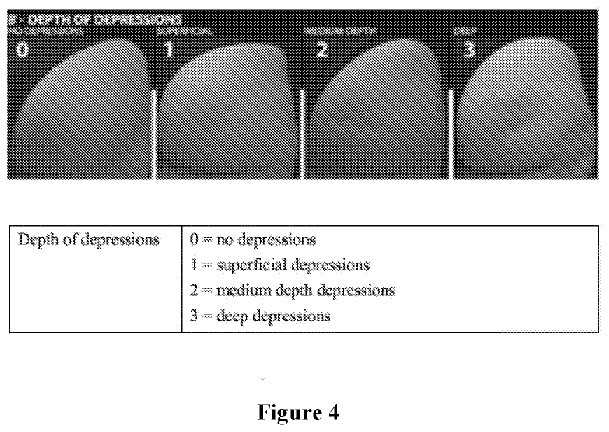

“Hexsel Depression Depth Score” as used herein means an assessment of only (B) depth of depressions from the Hexsel CSS (FIG. 4):

0=no depressions
1=superficial depressions
2=medium depth depressions
3=deep depressions.

“Images” or “Imagery” as used herein means photographs, illustrations, drawings, models, 3-D models, computer-generated images, MRI images and the like.

“Likert Scale score” as used herein means the score identified by an independent blinded assessor of the change in the treated area (buttock or thigh) at each post-treatment visit by comparing the photographs (2-D color, 3-D color and 3-D greyscale) of cellulite from the Day 1 pretreatment (Baseline) with photographs for the post-treatment visit. The score is captured in the following 5-point Likert Scale:

| −1 | 0 | 1 | 2 | 3 |
|---|---|---|---|---|
| Worse | No change | Improved | Much improved | Very much |
| | | | | improved |
| The treated area | The treated | Obvious | Marked | Optimal |
| appearance is | area | improvement in the | improvement in | cosmetic result |
| worse than | appearance is | treated area | the treated area | from treatment |
| before treatment | essentially the | appearance from | appearance from | of the treated |
| | same as before | before treatment, but | before treatment, | dimples |
| | treatment | additional treatment | but not completely | |
| | | is indicated | optimal | |

“Optional” or “optionally” means that the subsequently described element, component or circumstance may or may not occur, so that the description includes instances where the element, component, or circumstance occurs and instances where it does not.

“Patient Reported Cellulite Impact Scale (PR-CIS)” as used herein means an assessment of the visual and emotional impact of cellulite (happy, bothered, self-conscious, embarrassed, looking older, or looking overweight or out of shape) using a 6-question survey, with each question rated on a numerical rating scale from 0 (not at all) to 10 (extremely). More specifically, the PR-CIS is a 6-item static questionnaire assessing the visual and emotional impact of cellulite (happy, bothered, self-conscious, embarrassed, looking older or looking overweight or out of shape); each item is answered by the subject on an 11-level numerical rating scale from 0 (not at all) to 10 (extremely) while viewing digital images of their buttocks or thighs. This assessment may be of all thighs and/or buttocks together rather than each individual area separately. A PR-CIS total score can be derived from 6 individual questions:
Question 1—Thinking about the areas selected for treatment, how happy are you with the appearance of your cellulite?
Question 2—Thinking about the areas selected for treatment, how bothered are you by the appearance of your cellulite?
Question 3—Thinking about the areas selected for treatment, how self-conscious are you about the appearance of your cellulite?
Question 4—Thinking about the areas selected for treatment, how embarrassed are you about the appearance of your cellulite?
Question 5—Thinking about the areas selected for treatment, how much older do you look because of your cellulite?
Question 6—Thinking about the areas selected for treatment, how overweight or out of shape do you look because of your cellulite?

“Patient Reported Cellulite Impact Scale (Abbreviated)” (PR-CIS Abbreviated) as used herein means an assessment of the visual and emotional impact of cellulite (happy, bothered, self-conscious, embarrassed, or looking overweight or out of shape) using a 5-question survey, with each question rated on a numerical rating scale from 0 (not at all) to 10 (extremely). More specifically, the PR-CIS Abbreviated is a 5-item static questionnaire assessing the visual and emotional impact of cellulite (happy, bothered, self-conscious, embarrassed, or looking overweight or out of shape); each item is answered by the subject on an 11-level numerical rating scale from 0 (not at all) to 10 (extremely) while viewing digital images of their buttocks or thighs. This assessment may be of all thighs and/or buttocks together rather than each individual area separately. In a non-limiting example, a PR-CIS Abbreviated total score can be derived from 5 individual questions:
Question 1—Thinking about the areas selected for treatment, how happy are you with the appearance of your cellulite?
Question 2—Thinking about the areas selected for treatment, how bothered are you by the appearance of your cellulite?
Question 3—Thinking about the areas selected for treatment, how self-conscious are you about the appearance of your cellulite?
Question 4—Thinking about the areas selected for treatment, how embarrassed are you about the appearance of your cellulite?
Question 5—Thinking about the areas selected for treatment, how overweight or out of shape do you look because of your cellulite?
   A PR-CIS Abbreviated total score can be derived from other sets of 5 questions from the full PR-CIS.

“Patient Reported Photonumeric Cellulite Severity Scale (PR-PCSS)” as used herein are the photonumeric scales described in PCT Patent Application PCT/US2018/020551 (published as WO2018/160905 on Sep. 7, 2018) used by patients and designed to assess the severity of cellulite into 5 levels.

“Photonumeric” as used herein means using a series of photographs, illustrations, drawings, models, 3-D models, computer-generated images, MRI images, images and the like each assigned a different level of cellulite severity in a scale.

“Sequential visit” as used herein means two or more clinician visits or times where cellulite changes are assessed by a scale. The time between visits may be about two weeks, about one month, about two months, about three months, about fourth months, about five months, about six months, about one year, about two years, about three years, or about five years or longer.

“Serious Adverse Events” as used herein means an adverse event that results in death, is immediately life-threatening, results in or prolongs an inpatient hospitalization, results in permanent or substantial disability, is a congenital anomaly/birth defect, or is considered an important medical event.

The terms “subject” or “patient” is used interchangeably herein and refers to a human or other mammal.

“Subject Global Aesthetic Improvement Scale (S-GAIS)” and “Investigator Global Aesthetic Improvement Scale (I-GAIS)” as used herein mean the following scales to assess cellulite severity and/or improvement. The subject is asked the following introductory question: “How would you rate the appearance of your treated cellulite after treatment?” The rating ranges from −3 (Very much worse) to +3 (Very much improved) depending on the subject's response, as shown in Table 3.

TABLE 3
Subject Global Aesthetic Improvement Scale (S-GAIS) and
Investigator Global Aesthetic Improvement Scale (I-GAIS)

| Rating | Response Option | Description (S-GAIS) | Description (I-GAIS) |
|---|---|---|---|
| +3 | Very much improved | My treated cellulite looks very much | Optimal cosmetic result from |
| | | better. | treatment of the treated dimples |
| +2 | Much improved | My treated cellulite looks much | Marked improvement in the treated |
| | | better, but additional treatment | area appearance from |
| | | would slightly improve the result. | before treatment, but not completely |
| | | | optimal |
| +1 | Improved | My treated cellulite looks better, but | Obvious improvement in the treated |
| | | additional treatment is necessary. | area appearance from |
| | | | before treatment, but additional |
| | | | treatment is indicated |
| 0 | No change | My treated cellulite looks essentially | The treated area appearance is |
| | | the same as it did originally. | essentially the same as before |
| | | | treatment |
| −1 | Worse | My treated cellulite looks worse than | The treated area appearance is worse |
| | | it did originally. | than before treatment |
| −2 | Much worse | My treated cellulite looks much | Marked worsening in appearance |
| | | worse than it did originally. | from the initial condition |
| −3 | Very much worse | My treated cellulite looks very | Obvious worsening in appearance |
| | | much worse than it did originally. | from the initial condition |

“Subject Satisfaction with Cellulite Treatment” (SSCT) as used herein means a subject satisfaction rating ranging from −2 to +2. As an example, Table 4 below provides such assessment for cellulite treatment on the buttock. The patients are asked: “Today, how satisfied are you with the results of the cellulite treatment you received on the specific area or areas on your buttocks that were treated?” They then choose an answer/rating as shown in Table 4.

TABLE 4
Subject Satisfaction with Cellulite
Treatment Assessment - Buttocks

| Rating | Description |
|---|---|
| +2 | I am very satisfied with the cellulite treatment on my buttocks. |
| +1 | I am satisfied with the cellulite treatment on my buttocks. |
| 0 | I am neither dissatisfied nor satisfied with the cellulite |
| | treatment on my buttocks. |
| −1 | I am dissatisfied with the cellulite treatment on my buttocks. |
| −2 | I am very dissatisfied with the cellulite treatment on my |
| | buttocks. |

Table 5 provides such assessment for cellulite treatment on the thighs. The patients are asked: “Today, how satisfied are you with the results of the cellulite treatment you received on the specific area or areas on your thighs that were treated?” They then choose an answer/rating as shown in Table 5.

TABLE 5
Subject Satisfaction with Cellulite Treatment Assessment - Thighs

| Rating | Description |
|---|---|
| +2 | I am very satisfied with the cellulite treatment on my thighs. |
| +1 | I am satisfied with the cellulite treatment on my thighs. |
| 0 | I am neither dissatisfied nor satisfied with the cellulite |
| | treatment on my thighs. |
| −1 | I am dissatisfied with the cellulite treatment on my thighs. |
| −2 | I am very dissatisfied with the cellulite treatment on my thighs. |

“Subject Self-Rating Scale (SSRS)” as used herein is a scale used by a subject to assess his/her satisfaction with appearance in association with cellulite using whole numbers on a 7-level scale that ranges from 0 (extremely dissatisfied) to 6 (extremely satisfied) as shown in Table 6.

TABLE 6
Subject Self-Rating Scale (SSRS)

| Rating | Response Option |
|---|---|
| 6 | Extremely satisfied |
| 5 | Satisfied |
| 4 | Slightly satisfied |
| 3 | Neither satisfied nor dissatisfied |
| 2 | Slightly dissatisfied |
| 1 | Dissatisfied |
| 0 | Extremely dissatisfied |

The term “therapeutically effective amount,” as used herein, refers to the amount of collagenase needed to reduce the severity of cellulite in a patient or a statistically significant population of patients. The amount collagenase composition employed will be that amount necessary to deliver an amount of collagenase needed to achieve the desired result. In practice, this will vary depending upon the collagenase being injected, the injection technique, and the enzymatic activity at the treatment area.

The term “treatment-emergent adverse event” or “TEAE” as used herein is any condition that was not present prior to treatment with study medication but appeared following treatment, was present at treatment initiation but worsened during treatment, or was present at treatment initiation but resolved and then reappeared while the individual was on treatment (regardless of the intensity of the ΔE when the treatment was initiated).

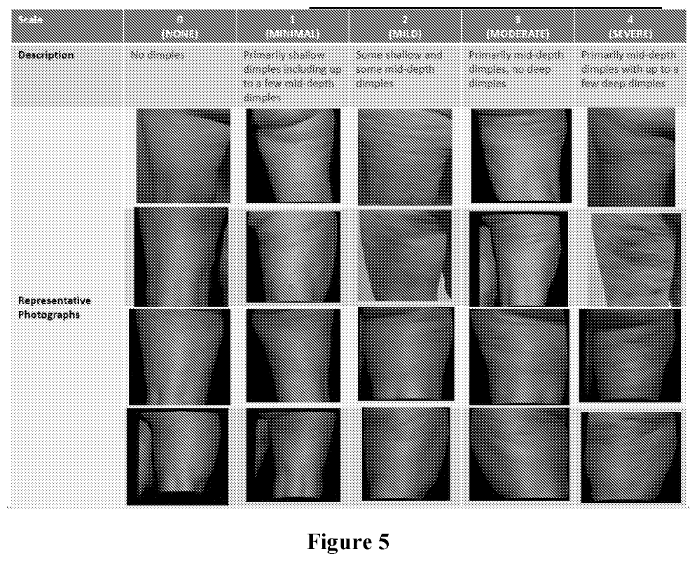

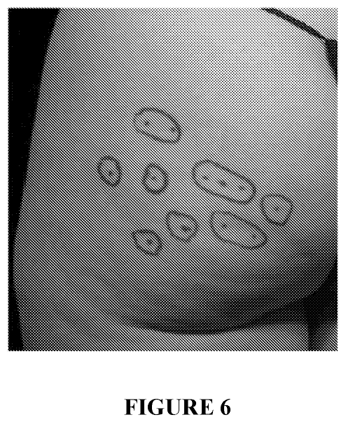

The term “Thigh Cellulite Efficacy Scale” or “TCES” as used herein means the photonumeric scale shown in FIG. 5 (or a substantially similar scale) used by clinicians and patients to assess thigh cellulite severity, improvement, and/or patient satisfaction and assist in assessing collagenase efficacy. The patient reported version is referred to as PR-TCES; the clinician reported version is referred to as CR-TCES.

The term “treatment visit” or “treatment” as used herein means one or more injections or treatments to affected area(s) with a therapeutically effective amount of at least one active agent useful in treating cellulite in a single office visit.

The terms “validated,” “validity” or “validation” as used herein mean a process by which a particular scale is demonstrated to be accurate and reliable, including the repeatability of visual assessments to ensure that the same result can be consistently obtained. Validation further examines the precision, accuracy and sensitivity of the scale to confirm the measurements taken by it are reliable, reproducible and robust.

### B. Introduction

The present disclosure relates to methods of treating cellulite, comprising the administration of a therapeutically effective amount of one or more collagenases to a subject having the appearance of cellulite, through the use of certain injection techniques described below.

Generally, there are four phases of treatment: (1) The clinician and patient perform pretreatment assessments to determine a pretreatment baseline, and the clinician selects dimples to be treated; (2) the clinician marks the dimples to be treated at the nadir, if a nadir is present; (3) the clinician treats the patient with collagenase; and (4) the clinician and patient perform post-treatment assessments. These phases are detailed below. The phases, and steps within them, are optional and the order of steps is not intended to be limiting as the order may vary yet achieve comparable results.

### C. Phase 1—Pretreatment Assessments

In a first phase of the methods of treating cellulite described herein, the clinician performs a selection of cellulite dimples to be treated based on the following criteria:
Dimples should be well-defined and evident naturally when the patient is standing in a relaxed pose (standing position with relaxed gluteus muscles) as confirmed by photographs
Dimples chosen should be the ones the clinician believes is most likely to improve aesthetic appearance of each entire buttock, thigh or other affected area
Photographs of affected areas are taken before treatment when the patient is standing in a relaxed pose
Before injection, an assessment is performed, i.e., the clinician and/or patient independently assess the photographs and score the result using one or more of the following scales or assessment methods:
   Hexsel Cellulite Severity Scale (Hexsel CSS)
   Hexsel Depression Depth Score
   Likert Scale
   Dimple Analysis
   Clinician Reported Photonumeric Cellulite Severity Scale (CR-PCSS)
   Patient Reported Photonumeric Cellulite Severity Scale (PR-PCSS)
   Investigator Global Aesthetic Improvement Scale (I-GAIS)
   Subject Global Aesthetic Improvement Scale (S-GAIS)
   Patient Reported Cellulite Impact Scale (PR-CIS)
   Subject Self-Rating Scale (SSRS)
   Subject Satisfaction with Cellulite Treatment (SSCT)
   Thigh Cellulite Efficacy Scale (PR-TCES; CR-TCES)
   Body-Q
   Fitzpatrick scale
   Any validated photonumeric or other scale used by clinicians and/or patients to assess cellulite severity, improvement, and/or patient satisfaction

### D. Phase 2—Marking of Dimples to be Treated

In a second phase of the treatments described herein, dimples to be treated can be marked with a dot(s) by the clinician. More photographs may be taken.

### E. Phase 3—Collagenase Injections

In a third phase of treatment, a clinician treats the patient with collagenase injections.

1. Types of Collagenases

The collagenases useful in the present disclosure include any of the collagenases as defined above. By way of further background, matrix metalloproteinases (MMPs) can be comprised of collagenases falling within the definition herein. For example, MMP-1 comprises collagenase 1; MMP-8 comprises collagenase 2/neutrophil collagenase; MMP-13 comprises collagenase 3; and MMP-18 comprises collagenases 4. Further, cathepsins can be classified as collagenases.

2. Collagenase Enzyme Kinetics

Collagenases useful in the present disclosure may also be characterized by their enzyme kinetics. Here, the approximate kinetic values of the one or more collagenases effective to treat cellulite include the following:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM^{31 1}sec⁻¹ of about 5,140 to 508,814 (SRC assay), or about 60 to 5,934 (GPA assay)
   Vₘₐₓ=maximal rate
   K_{M}=[Substrate] at 50% of Vₘₐₓ
   K_{cat}=molecules of substrate cleaved per second
   1/K_{cat}=The microseconds required to cleave a molecule of substrate.
   These values may be determined experimentally using the microplate assays described below, but with different substrates and times. Other assays and parameters may be employed.

These values reflect a quantitative expression of enzyme behavior based on the Michaelis-Menten Equation:

$$\frac{{V}_{0} = {V}_{max} [ S ]}{{K}_{M} + [ S ]}$$

Wherein V₀ is the reaction rate (velocity) at a substrate concentration [S], Vₘₐₓ is the maximum rate that can be observed, and K_{M} is the Michaelis constant, which correlates to the concentration of substrate that yields 50% of Vₘₐₓ.

$$\frac{{K}_{M} = {k}_{2} + {k}_{- 1}}{{k}_{1}}$$

Wherein k₁, k₋₁ and k₂ are rate constants for the following steps:

$$E + S {{k}_{1}}_{{↔}_{{k}_{- 1}}} ES {{k}_{2}}_{\to } E + P$$

Wherein E is the enzyme, S is the substrate, ES is the enzyme-substrate complex, and P is the product.

The catalytic constant K_{cat} refers to the turnover number, i.e., how fast the ES complex proceeds to E+P. It reflects the number of catalytic cycles that each active site undergoes per unit time.

In certain embodiments, AUX-I and AUX-II have the following characteristics:
AUX I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
AUX II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Assumptions:

K_{cat}=Vₘₐₓ/[AUX]=(nmoles Substrate/nG AUX*min−1)/nG AUX

Catalytic efficiency (K_{cat}/K_{M}) generally represents the enzyme's overall ability to convert substrate to product, and reflects both binding and catalytic events. In another embodiment, AUX-I and AUX-II comprise the following characteristics.

| | AUX-I | AUX-II |
|---|---|---|
| | (SRC | (GPA |
| | assay)* | assay)+ |
| Vₘₐₓ, min⁻¹ | 3.8 | 15.4 |
| K_{M}, mM | 2.07 × 10⁻⁴ | 1.5 |
| K_{cat}, sec⁻¹ | 53 | 4,636 |
| 1/K_{cat}, | 18,799 | 216 |
| microseconds | | |
| K_{cat}/K_{M}, mM⁻¹ sec⁻¹ | 256,977 | 2,997 |

Vₘₐₓ = maximal rate
K_{M} = [Substrate] at 50% of Vmax
K_{cat} = molecules of substrate cleaved per second
1/K_{cat} = The time required to cleave one molecule of substrate
K_{cat}/K_{M} is often used to represent catalytic efficiency of the enzyme
*By SRC microplate assay
+By GPA microplate assay

3. Potency (Specific Activity) of Collagenase(s)

Assays have been developed and used to determine the specific activity (potency) of collagenase. Such assays are described in subsections a. to c. and characterize collagenase by its ability to convert substrate to product within a given time period with a pre-determined enzyme concentration. In certain non-limiting embodiments, these assays are used to determine the potency of each of AUX-I and AUX-II, and the combined CCH drug product (1:1 ratio of AUX-I and AUX-II). The SRC assays (described below) use collagen as substrate for the reaction. The SRC assays use soluble rat (tail) collagen (SRC) as substrate, and are used to measure Type I collagenases activity, with Type II collagenases contributing approximately 20% of the observed activity of a collagenase mixture. The SRC assay is fluorometric and utilizes fluorescamine to detect the peptides produced by the Type I digestion of SRC. The reaction is run at pH 7.2 in 4-(2-hydroxyethyl)-1-piperazineethanesulfonic acid (HEPES) buffer containing 15 mM divalent calcium ion for 2.5 h at 25° C.

The bovine tendon collagen (BTC) assay (described below) is based on the procedures of Mandl et al., Arch. Biochem. Biophys. 74: 465-475 (1958), as modified by Keller and Mandl, Arch. Biochem. Biophys. 101: 81-88 (1963). See also Rosen, Arch. Biochem. Biophys. 67: 10-15 (1957). The BTC assay uses insoluble bovine tendon collagen as substrate and measures both Type I and Type II activity (such as AUX-I and II collagenases). The BTC assay is colorimetric and utilizes ninhydrin to detect the peptides produced by Type I and Type II degradation of BTC. This reaction is also run at pH 7.2, but for 22 h at 37° C. in tris (hydroxymethyl) aminomethane (TRIS) buffer containing 10 mM divalent calcium ion.

The third collagenase type of assay, the GPA assays (described below), utilize a soluble, derivatized hexapeptide (carbobenzoxy-GPGGPA) as substrate. The GPA assay is used to measure primarily Type II activity, with Type I contributing approximately 10% of observed activity. Type II collagenase cleaves the hexapeptide into two tripeptides, one of which (GPA) has a free amino terminus which reacts with fluorescamine to provide a fluorescent product. The GPA assay is run at pH 7.2 in HEPES buffer containing 100 mM divalent calcium ion for 10 min at 25° C.

The SRC and BTC assays both degrade a natural substrate (collagen), which more closely approximates what collagenase injection is designed to do therapeutically. The GPA assays have the advantage that they utilizes a well-defined, small molecular weight hexapeptide as substrate and two well-defined tripeptides are produced. The GPA assays produce a fluorescent signal and is quite sensitive. Finally, the GPA assay are amenable to Michaelis-Menten kinetic analysis because it uses a single substrate, and reaction conditions (10 minutes incubation), which approximate initial enzyme velocities. The SRC assay is well-suited to collagen-degrading enzymes with collagen binding domains, whereas the GPA assay is well-suited to collagen-degrading enzymes without collagen binding domains, which are often referred to as gelatinases.

a. GPA Unit Assay Methods and Specific Activity Units

i. Collagenase Potency as Measured by GPA Assay (Cuvette)

The GPA assay is primarily used to measure the potency of a class II collagenase. The first step of the assay involves an enzymatic reaction involving the digestion of the substrate carbobenzoxy-glycyl-L-prolyl-glycyl-glycyl-prolyl-L-alanine (zGPGGPA) by a collagenase sample into two peptides: carbobenzoxy-glycyl-L-prolyl-glycine (zGPG) and glycyl-prolyl-L-alanine (GPA). The second step involves the subsequent measurement of liberated GPA with the fluorogenic derivative fluorescamine. The assay follows the methodology below, but a person of ordinary skill in the art will appreciate that certain modifications (e.g., dilution concentrations and times) may be made yet carry out the purpose of the assay.

The general methodology is as follows. Leucine standards are prepared. A collagenase sample is obtained and solutions are prepared to be used in the first step for the enzymatic cleavage of zGPGGPA (hereafter “substrate”) by collagenase. Following this step, the collagenase-treated samples (containing the liberated GPA) and leucine standards are treated at room temperature for a period of time with fluorescamine in order to fluorescently tag the free amino groups of the generated GPA and leucine molecules, respectively. The fluorescence emission of each solution at 480 nm is measured following excitation at 392 nm. The resulting slopes of the leucine and collagenase sample curves are then used to calculate potency units as follows:

Potency (f-GPA units/mg)=(M_{Sample}/M_{Leucine})×(DF/T)

Where:

Mₛₐₘₚₗₑ=Slope of the collagenase sample potency curve

M_{Leucine}=Slope of the leucine standard curve

DF=Dilution Factor

T=Reaction time

Additional, non-limiting details regarding the GPA assay methodology are set forth below.

Buffers and Reagents

1. f-Appel's Buffer, pH 7.2 (55 mM HEPES, 100 mM calcium acetate)

2. 1 mM Leucine Working Stock Solution

3. 200 mM Borate, pH 9.0

4. 0.5 mM Fluorescamine Solution in Acetone

5. 2 mg/mL zGPGGPA Substrate in f-Appel's Buffer

Solution Preparation

Solutions are prepared as follows:

f-Appel's Buffer: Dissolve 13.0 g HEPES and 17.6 g calcium acetate in approximately 800 mL of water. Adjust pH to 7.2 with sodium hydroxide and QS to 1 L with water. Store at 2-8 degrees C.

10 mM Leucine Stock Solution: Dissolve 65.5 mg of leucine in 50 mL of water. Leucine must be weighed directly into a 100 mL (or equivalent) glass beaker on the scale. Weigh out approximately 65 mg (target weight) of leucine into the beaker. Based on the weight of leucine weighed, calculate the amount of water to add to the beaker using the equation below. Add the calculated volume of water to the beaker and mix thoroughly to ensure the leucine is fully dissolved. Dispense in to 1 mL aliquots. Store at less than or equal to 20 degrees C.

$$V2 ( mL ) = C 2 ( mg ) \times \frac{V 1 ( 50 mL )}{C 1 ( 65.5 mg )}$$

Where:
C2=mass of leucine weighed (mg)
V1=50 (mL of water)
C1=65.5 (mg of leucine)
V2=volume of water needed to produce a 10 mM stock solution (mL)

1 mM Leucine Working Stock Solution: Thaw a vial of 10 mM Leucine Stock Solution and dilute to 1 mM by combining 150 μL with 1350 μL water. Mix well prior to use.

0.5 N HCl: Dilute HCl to 0.5 N with water and mix well. Store at room temperature. Alternatively, commercially available 0.5 N HCl may be used.

200 nM Borate, pH 9.0: Dissolve 2.4 g boric acid in approximately 150 ML water. Adjust the pH to 9.0 using sodium hydroxide. QS to 200 mL with water and mix well. Store at 2-8 degrees C.

0.5 mM Fluorescamine Solution: Mix 15 mg of fluorescamine with 100 mL acetone and swirl to dissolve. Store at 2-8 degrees C. protected from light.

Substrate Solution (2 mg/mL zGPGGPA): Prepare substrate at 2 mg/mL with f-Appel's buffer. Dissolve on a mechanical shaker/rotator, allowing sufficient time for complete dissolution (about 15 minutes).

Leucine Standard Curve

The leucine standard curve is prepared according to Table 7.

TABLE 7
Preparation of the leucine standard curve.

| Standard | L1 | L2 | L3 | L4 | L5 | L6 |
|---|---|---|---|---|---|---|
| Leucine Conc. (μM) | 0 | 70 | 140 | 210 | 280 | 350 |
| Water (μL) | 500 | 430 | 360 | 290 | 220 | 150 |
| 0.5N HCl (μL) | 500 | 500 | 500 | 500 | 500 | 500 |
| 1 mM Leucine (μL) | 0 | 70 | 140 | 210 | 280 | 350 |

“L1” means Leucine standard sample 1.

100 μL of each Leucine Standard is then transferred into separate tubes for detection of fluorescamine.

Collagenase Sample Preparation

The collagenase sample is diluted to 0.01 mg/mL with f-Appel's Buffer in two stages and vortexed gently to mix. The following is an example dilution scheme:

1. 100 μL×1.0 mg/mL→1000 μL=0.1 mg/mL

2. 100 μL×0.1 mg/mL→1000 μL=0.01 mg/mL

Blank Preparations

Blanks are prepared by combining 45 μL of the diluted preparation with 500 μL of 0.5 N hydrochloric acid to inactivate the enzyme. Add 455 μL of zGPGGPA substrate solution and vortex to mix thoroughly. Transfer 100 μL of each blank into separate tubes for detection of impurities that may react with fluorescamine.

Potency Curves

A set of potency curves are prepared for each collagenase sample as follows:

| | 2 mg/mL Substrate | f-Appel's |
|---|---|---|
| Tubes | Solution | Buffer (μL) |
| 1-2 | 1 | 45 |
| 3-4 | 1 | 30 |
| 5-6 | 1 | 15 |

Warm the tubes containing substrate and buffer in a water bath at 25 degrees C. for a minimum of 15 minutes. Label a second set of tubes and add 50 μL of 0.5 N hydrochloric acid to each. Add the diluted collagenase sample preparations (0.01 mg/mL) to the tubes according to Table 8 for a 10 minute incubation, mix and return to the 25 degrees C. water bath. Start the incubation period upon addition of the first preparation to the pre-warmed substrate.

TABLE 8
Sample Preparation

| Preparation | Tubes | Sample (μL) |
|---|---|---|
| Potency Curve | 1-2 | 55 |
| Potency Curve | 3-4 | 70 |
| Potency Curve | 5-6 | 85 |

Remove the preparations from the water bath with 1-2 minutes remaining on the 10 minute incubation and vortex gently to mix. Ten minutes after addition of the first preparation to the substrate, transfer 50 μL from each tube into the tubes containing 50 μL of 0.5 N HCl. The preparations should be added directly to the acid to quench the digestion. Vortex each tube to mix well after quenching all preparations.

Detection

Add 400 μL of 200 mM Borate Buffer and 500 μL of 0.5 mM Fluorescamine Solution to all detection tubes containing 100 μL of each preparation (blanks, collagenase sample potency curves, and leucine standards). Vortex thoroughly to mix. Allow the tubes to incubate at room temperature for a minimum of 10 minutes.

Fluorometer Setup

Set up the fluorometer with the following instrument parameters and read the fluorescence of each preparation with 1 hour of derivatization.

| Parameter | Setting | |
|---|---|---|
| Excitation Wavelength | 392 | nm |
| Emission Wavelength | 480 | nm |
| Integration | 5.0 | sec. |
| Slits (Ex & Em) | 5.0 | nm (band pass) |
| Path Length | 3 | mm |

Calculations

Plot the concentration of each leucine standard (X-axis) against the fluorescence response at 480 nm (Y-axis). Determine the slope (m) and coefficient of determination (R²). Determine the mean fluorescence of each potency curve preparation. Prepare collagenase sample and leucine potency curves by plotting the concentration of each preparation (X-axis) against the mean fluorescent response at 480 nm (Y-axis). Determine the slope (m) and coefficient of determination (R²) for the resulting linear curves.

Determine Potency of Collagenase Sample

Potency (f-GPA units/mg)=(Mₛₐₘₚₗₑ/M_{Leucine})×(DF/T)

Where:

Mₛₐₘₚₗₑ=Slope of the collagenase sample potency curve

M_{Leucine}=Slope of the leucine standard curve

DF=Dilution Factor (1100 μL/50 μL=22)

T=Reaction time (10 minutes)

ii. GPA Microplate Assay for the Determination of Class II Collagenase Activity in a Collagenase Sample

This method is similar to the GPA assay above, except is performed in a microplate. Like the assay above, the microplate assay measures the proteolytic activity of collagenase samples in the enzymatic cleavage of the substrate carbenzoxy-glycyl-L-prolyl-glycyl-glycyl-L-propyl-L-alanine (zGPGGPA) (hereafter, “substrate”). The assay follows the methodology below, but a person of ordinary skill in the art will appreciate that certain modifications (e.g., dilution concentrations and times) may be made yet carry out the purpose of the assay.

Reagents

1. Peptide substrate (zGPGGPA) (Bachem M1260 or equivalent)

2. Tripeptide GPA (Bachem H3615 or equivalent)

3. Fluorescamine (Acros 191675000 or equivalent)

4. Purified Water (Milli-Q-Plus 18.2 MΩ system or equivalent)

5. 1 M HEPES buffer (Gibco 15630-080 or equivalent)

6. Surfact-Amps 20™ (10% Tween solution) (Pierce Cat.#28320 or equivalent)

7. 1 M Calcium Acetate (Ca(C₂H₃O₂)₂) (Emerald Biosciences Cat.#EBS-100-CAAC or equivalent)

8. Boric acid (Sigma B7660 or equivalent)

9. 2.5 N NaOH (J.T Baker 5666-02 or equivalent)

10. 0.5 N Hydrochloric Acid (VWR 101223-134 or equivalent)

11. Acetone (Sigma 270725 or equivalent)

Preparation of Solutions

(i) Preparation of assay buffer (50 mM HEPES pH 7.1/0.05% Tween 20/5 mM (Ca(C₂H₃O₂)₂): An amount of 50 mL 1 M HEPES is pipetted into 800 mL DI water. 5 mL 1 M (Ca(C₂H₃O₂)₂) and 5 mL Surfact-Amps (10% Tween 20) are added. The pH is checked and adjusted to 7.1±0.05 if necessary. A sufficient quantity of water is added to adjust the volume to 1 L and the solution is filtered through a 0.22 micron filter. This assay buffer can be stored at room temperature for up to 3 months.

(ii) Preparation of 0.1 N NaOH: An amount of 2 mL of 2.5 N NaOH is added into 48 mL DI water. This solution can be stored at room temperature for up to 3 months.

(iii) Preparation of 4 mg/mL tripeptide GPA stock solution: An amount of 400 mg (±1 mg) of tripeptide GPA is dissolved into 10 mL 0.1 N NaOH and vortexed until totally dissolved. A sufficient quantity of assay buffer is added to make the volume 100 mL and the solution is dispensed into 0.5 mL aliquots and stored at −70° C. The 4 mg/mL tripeptide GPA stock can be stored at −70° C. for up to one year.

(iv) Preparation of 4 mg/mL (6.8 mM) peptide substrate zGPGGPA: An amount of 400 mg (+1 mg) of the peptide substrate zGPGGPA is dissolved into 10 mL 0.1N NaOH, vortex until totally dissolved. A sufficient quantity of assay buffer is added to make the volume 100 mL. This solution can be stored at 4° C. for up to 3 months.

(v) Preparation of 120 mM Boric Acid pH 9.0: An amount of 7.4 g (±0.5 g) of the boric acid is dissolved into 800 mL DI water. The solution is titrated with NaOH to pH 9.0. a sufficient quantity of DI water is added to adjust the volume to 1 liter. This solution can be stored at room temperature for up to 3 months.

(vi) Preparation of 1 mM Fluorescamine in Acetone: An amount of 28±2 mg Fluorescamine is dissolved in 100 mL acetone. This solution needed to be freshly prepared and protected from light and moisture.

Preparation of Tripeptide GPA Standard and Serial Dilution

A 0.08 mg/mL (329 μM) tripeptide GPA standard is prepared by making a 50-fold dilution of the 4 mg/mL tripeptide GPA stock in assay buffer (for example, 20 μL 4 mg/mL tripeptide GPA in 980 μL assay buffer). In the assay plate, row A, 200 μL of 329 μM tripeptide GPA standard is pipetted into A1 and A7. An amount of 100 μL assay buffer is pipetted into A2-A6 and A8-A12.

For the tripeptide GPA standard serial dilution, an amount of 100 μL is transferred from A1 into A2, mixed, an amount of 100 μL is transferred from A2 into A3, and repeated until A5. An amount of 100 μL is taken out from A5 well so that its final volume is 100 μL. The A6 well contains buffer only.

For the second tripeptide GPA standard serial dilution, an amount of 100 μL is transferred from A7 into A8, mixed, an amount of 100 μL is transferred from A8 into A9, and repeated until well A11. An amount of 100 μL is taken out from A11 well so that its final volume is 100 μL. The A12 well contains buffer only.

Preparation of Collagenase Samples

For collagenase samples (e.g., a lyophilized collagenase drug product), the sample is allowed to come to room temperature for at least 10 minutes and reconstituted to form a 500 ng/mL stock solution. Different concentrations may be used. A test collagenase sample (T1A) is prepared from the stock solution by diluting with assay buffer. The procedure is repeated to prepare triplicate test samples (T1A, T1B, T1C).

Discussion

In this method, 50 μL of increasing concentrations of the collagenase test samples are mixed with 50 μL of excess substrate (2.0 mg/mL final concentration) in a 96-well plate. An amount of 50 μL of assay buffer is added to rows C-G in a U-bottomed, 96 well polypropylene reaction plate. 150 μL of collagenase samples are pipetted into row B. Then, a 1/1.5 serial dilution is performed using a multi-channel pipette, by transferring 100 μL of collagenase sample from row B into row C, mixing and repeating the process until row G is reached. An amount of 100 μL is removed and discarded from row G. Table 9 contains the final collagenase concentrations after adding 50 μL substrate to row B through row H.

The Blank is prepared in row H by pipetting 50 μL assay buffer to row H. This row contains no enzyme. Exemplary concentrations are shown in Table 9.

TABLE 9
Assay Target Concentrations After Substrate Addition

| | | Collagenase |
|---|---|---|
| Row | Dilution | (ng/ml) |
| A | N/A | N/A |
| B | Stock | 250 |
| C | 1/1.5 | 167 |
| D | 1/2.3 | 111 |
| E | 1/3.4 | 74 |
| F | 1/5.1 | 49 |
| G | 1/7.6 | 33 |
| H-Blank | N/A | 0 |

Collagenase Reaction

The zGPGGPA substrate is cleaved by class II collagenases into zGPG and GPA during a 15 minute incubation time at room temperature. The incubator and temperature probe are turned on (temperature 22±1° C. prior to the addition of substrate to the plates). To column 1-12 in row B-H, 50 μL 4 mg/mL (6.8 mM) zGPGGPA substrate is added column by column, then mixed. The reaction start time begins after the substrate is added to the first column. The plate is covered and placed in the 22±1° C. incubator for a total reaction time of 15±1 minutes.

After incubation, the reaction is quenched by the addition of hydrochloric acid, and the amount of released GPA peptide is quantitated after reacting the free amino terminus of the peptide with the fluorogenic reagent, fluorescamine. To quench the reaction, 100 μL of 0.5 N HCl is added into each well from row A to row H, added column by column, and then mixed. Reaction time ends after the HCl is added to the first column.

Detection

An amount of 195 μL of 120 mM Borate pH 9.0 is added to each well of a Microplate Greiner polypropylene black reading plate. 30 μL of the quenched reaction mixture is transferred from the reaction plate into the corresponding wells of the reading plate and mixed well. Then, 75 μL of 1 mM Fluorescamine is added to each well of the reading plate (using a polypropylene tray to dispense Fluorescamine/acetone) and mixed immediately after every addition. The plate is read within 15 minutes after Fluorescamine addition with a Molecular Devices M2 fluorescence plate reader using the following settings: Excitation 380 nm, Emission 473 nm, cutoff 455 nm, 6 reads/well, PMT medium.

The concentration of GPA (μM) versus the emission at 473 nm and the concentration of collagenase (ng/mL) versus the emission at 473 nm are plotted. For each plot, a linear regression is fitted with no fixed parameters. For collagenase test samples, the zero point data are excluded from the linear fit and the entire triplicate data set for each sample is used to generate the plot. The slopes for the tripeptide GPA standard and collagenase samples are determined.

Potency Determination

The collagenase sample specific activity can by calculated as follows:

GPA Microplate Assay Units=((Slope of Collagenase Sample)/(Slope of Tripeptide GPA×incubation time))×10⁶.

The specific activity of the collagenase test sample is determined from the slope of the tripeptide GPA standard and calculated by the curve-fitting program. Using the microplate method, different concentrations of substrate and different times may be used to calculate enzyme kinetics according to Michaelis-Menton.

iii. Collagenase Potency as Measured by GPA Assays

The collagenases useful in the present disclosure may have a potency of about 100,000 to about 300,000 GPA units/mg, or about 175,000 to about 300,000 f-GPA units/mg. In other embodiments, the potency may be about 70,000 to about 400,000 GPA units/mg, or about 100,000 to about 375,000 GPA units/mg, or about 125,000 to about 350,000 GPA units/mg, or about 150,000 to about 325,000 GPA units/mg, or about 175,000 to about 300,000 GPA units/mg, or about 200,000 to about 275,000 GPA units/mg. Alternatively, the potency may be about 70,000 to about 400,000 f-GPA units/mg, or about 100,000 to about 375,000 f-GPA units/mg, or about 125,000 to about 350,000 f-GPA units/mg, or about 150,000 to about 325,000 f-GPA units/mg, or about 175,000 to about 300,000 f-GPA units/mg, or about 230,000 to about 430,000 f-GPA units/mg, or about 200,000 to about 275,000 f-GPA units/mg. The collagenases may also have a potency of about 30,100 to 87,100, or about 43,000 to 67,000 GPA Microplate Assay Units. The above GPA assays may be employed to analyze the specific activity of any collagenase.

b. SRC Unit Assay Methods and Specific Activity Units

i. Collagenase Potency as Measured by SRC Assay (Cuvette)

The SRC assay is primarily used to measure the potency of a class I collagenase. The general methodology is as follows. Leucine standards and collagenase sample solutions are prepared. The first step of the assay involves an enzymatic reaction involving the digestion of soluble rat-tail tendon collagen (SRC) by the collagenase. The second step involves the subsequent measurement of liberated peptide fragments/amino acids with the fluorogenic derivative fluorescamine. The assay follows the methodology below, but a person of ordinary skill in the art will appreciate that certain modifications (e.g., dilution concentrations and times) may be made yet carry out the purpose of the assay.

Such collagenase and leucine standard samples are treated with reagents in order to tag the generated GPA with fluorescamine. The leucine standards and collagenase samples are allowed to incubate at room temperature for 10 minutes prior to determining the fluorescence of each solution at 392 and 480 nm excitation and emission wavelengths, respectively. The resulting slopes of the leucine and collagenase sample curves are then used to calculate potency units as follows:

Potency (f-SRC units/mg)=(Mₛₐₘₚₗₑ/M_{Leucine})×(DF/T)×CF

Where:

Mₛₐₘₚₗₑ=Slope of the collagenase sample potency curve

M_{Leucine}=Slope of the leucine standard curve

DF=Dilution Factor (1500 μL/100 mL=15)

T=Reaction time (2.5 hr×60 min/1 hr=150 min)

CF=Conversion factor (1000 μg/1 mg=1000)

Additional, non-limiting details regarding the SRC assay methodology are set forth below.

Buffers and Reagents
1. F-TC Assay Buffer, pH 7.2 (22 g HEPES [4-(2-Hydroxyethyl)-1-piperazineethanesulfonic acid], 4.4 g calcium acetate)
2. F-Enzyme Buffer, pH 7.2
3. 200 mM Borate, pH 9.0
4. 10 mM Leucine Stock Solution
5. 1 mM Leucine Working Stock Solution
6. 1 mM Fluorescamine Solution in Acetone
7. 2 mg/mL Rat Tail Collagen in 0.02N acetic acid

Solution Preparation

Solutions are prepared as follows:

F-TC Assay Buffer: Dissolve 22 g HEPES and 4.4 g calcium acetate in approximately 900 mL of water. Adjust pH to 7.2 with sodium hydroxide and QS to 1 L with water. Store at 2-8° C.

F-Enzyme Buffer: Dilute F-TC Assay Buffer by combining 4 mL with 16 mL water. Store at 2-8° C.

10 mM Leucine Stock Solution: Dissolve 65.5 mg of leucine in 50 mL of water. Leucine must be weighed directly into a 100 mL (or equivalent) glass beaker on the scale. Weigh out approximately 65 mg (target weight) of leucine into the beaker. Based on the weight of leucine weighed, calculate the amount of water to add to the beaker using the equation below. Add the calculated volume of water to the beaker and mix thoroughly to ensure the leucine is fully dissolved. Dispense in to 1 mL aliquots. Store at less than or equal to −20° C.

V2(mL)=C2(mg)×V1(50 mL)

C1(65.5 mg)
Where:
C2=mass of leucine weighed (mg)
V1=50 (mL of water)
C1=65.5 (mg of leucine)
V2=volume of water needed to produce a 10 mM stock solution (mL)

1 mM Leucine Working Stock Solution: Thaw a vial of 10 mM Leucine Stock Solution and dilute to 1 mM by combining 150 μL with 1350 μL water. Mix well prior to use.

0.5 N HCl: Dilute HCl to 0.5 N with water and mix well. Store at room temperature. Alternatively, commercially available 0.5 N HCl may be used.

0.02 N Acetic Acid: Combine 1 mL of 1 N Acetic Acid with 49 mL of water and mix well. Store at room temperature.

200 mM Borate, pH 9.0: Dissolve 2.4 g boric acid in approximately 150 mL water. Adjust the pH to 9.0 using sodium hydroxide. QS to 200 mL with water and mix well. Store at 2-8° C.

1 mM Fluorescamine Solution: Dissolve 15 mg of fluorescamine with 50 mL acetone and swirl to dissolve. Store at 2-8° C. protected from light.

Substrate Solution (2 mg/mL Rat Tail Collagen): Dilute sock rat tail collagen to 2 mg/mL with 0.02 N acetic acid. Store at 2-8° C.

Leucine Standard Curve

The leucine standard curve is prepared according to Table 10.

TABLE 10
Preparation of the leucine standard curve

| Reagent | L1 | L2 | L3 | L4 | L5 | L6 |
|---|---|---|---|---|---|---|
| Leucine Conc. (μM) | 0 | 70 | 140 | 210 | 280 | 350 |
| Water (μL) | 1000 | 930 | 860 | 790 | 720 | 650 |
| 1 mM Leucine (μL) | 0 | 70 | 140 | 210 | 280 | 350 |

100 μL of each Leucine Standard is then transferred into separate centrifuge tubes for detection of fluorescamine.

Collagenase Sample and Blanks Preparation

The sample is diluted to 0.01 mg/mL with F-Enzyme Buffer in two stages vortexed gently to mix. The following is an example dilution scheme:

1. 100 μL×1.0 mg/mL 4 1000 μL=0.1 mg/mL

2. 100 μL×0.1 mg/mL 4 1000 μL=0.01 mg/mL

Maintain the diluted samples at room temperature until use.

Blanks are prepared according to Table 11 by first combining the sample and 0.5 N hydrochloric acid to inactivate the enzyme prior to addition of buffers and substrate.

Collagenase samples in labeled tubes according to Table 11. Tubes 1, 2, 4 and 6 are prepared from one preparation and tubes 3, 5 and 7 from the duplicate preparation.

TABLE 11
Blank and Collagenase Sample Preparations

| | | 0.5N | F-Enzyme | F-TC | 2 mg/mL | |
|---|---|---|---|---|---|---|
| | | HCl | Buffer | Buffer | RTC | Sample |
| Preparation | Tube(s) | (μL) | (μL) | (μL) | (μL) | (μL) |
| Blank | 1 | 750 | 137.5 | 375 | 187.5 | 50.0 |
| Potency | 2-3 | — | 167.5 | 375 | 187.5 | 20.0 |
| Curve | 4-5 | — | 152.5 | 375 | 187.5 | 35.0 |
| | 6-7 | — | 137.5 | 375 | 187.5 | 50.0 |

The tubes are capped and vortexed gently to mix. The potency curve preparations are incubated in a 25° C.±3° C. water bath for 2.5 hours. At the end of incubation, the potency curve tubes are removed from the water bath. 750 μL of 0.5 N HCl is added to each preparation and vortexed thoroughly to mix. The preparations may be stored at 2-8° C. for up to 22 hours prior to detection.

Detection/Fluorometer Setup

The leucine standards are prepared as described above.

Set up the luminescence spectrometer with the following instrument parameters and read the fluorescence of each preparation with 1 hour of derivatization.

| Parameter | Setting | |
|---|---|---|
| Excitation Wavelength | 392 | nm |
| Emission Wavelength | 480 | nm |
| Integration | 5.0 | sec. |
| Slits (Ex & Em) | 5.0 | nm (band pass) |
| Path Length | 3 | mm |

Calculations

Plot the concentration of each leucine standard (X-axis) against the fluorescence response at 480 nm (Y-axis). Determine the slope (m) and coefficient of determination (R²). Do not force through zero. Determine the mean fluorescence of each duplicate preparation. Calculate the net fluorescence of each collagenase sample preparation.

F(net)=Mean Collagenase Sample (EM₄₈₀)−Blank (EM₄₈₀)

Plot the amount of the collagenase sample in each preparation (X-axis) against the net fluorescence (Y-axis). Determine the slopes (m) and coefficient of determination (R²). Do not force through zero.

Determine Potency of Collagenase Sample

Potency (f-SRC units/mg)=(Mₛₐₘₚₗₑ/M_{Leucine})×(DF/T)×CF
Where:
Mₛₐₘₚₗₑ=Slope of the collagenase sample potency curve
M_{Leucine}=Slope of the leucine standard curve
DF=Dilution Factor (1500 μL/100 mL=15)
T=Reaction time (2.5 hr×60 min/1 hr=150 min)
CF=Conversion factor (1000 μg/1 mg=1000)

The above SRC assay may be employed to analyze the specific activity of any collagenase.

ii. SRC Microplate Assay for the Determination of Class I Collagenase Activity in a Collagenase Sample

This method is similar to the SRC assay above, except is performed in a microplate. Like the SRC assay above, the microplate assay measures the collagenase activity towards soluble rat-tail collagen (SRC) substrate (hereafter, “substrate”). The assay follows the methodology below, but a person of ordinary skill in the art will appreciate that certain modifications may be made yet carry out the purpose of the assay.

Reagents
1. Soluble Rat Collagen Substrate (BD Biosciences 354236)
2. Tripeptide GPA (Bachem H3615 or equivalent)
3. Fluorescamine (Acros 191675000 or equivalent)
4. Purified Water (Millipore, Milli-Q-Plus 18.2 MΩ system or equivalent)
5. 1 M HEPES buffer (Gibco 15630-080 or equivalent)
6. 1 M Calcium Acetate (Ca(C₂H₃O₂)₂) (Emerald Biosciences EBS-100-CAAC or equivalent)
7. Surfact-Amps 20™ (10% Tween solution) (Pierce Cat.#28320 or equivalent)
8. 1.0 N Acetic acid (Sigma 318590 or equivalent)
9. 0.5 N Hydrochloric Acid (VWR 101223-134 or equivalent)
10. Boric acid (Sigma B7660 or equivalent)
11. 2.5 N Sodium hydroxide (J.T Baker 5666-02 or equivalent)
12. Acetone (Sigma 270725 or equivalent)

Preparation of Solutions

(i) Preparation of assay buffer (50 mM HEPES pH 7.1/0.05% Tween 20/5 mM (Ca(C₂H₃O₂)₂): An amount of 50 mL 1 M HEPES is pipetted into 800 mL DI water. 5 mL 1 M (Ca(C₂H₃O₂)₂) and 5 mL Surfact-Amps (10% Tween 20) are added. The pH is checked and adjusted to 7.1±0.1 if necessary. A sufficient quantity of water is added to adjust the volume to 1 L and the solution is filtered through a 0.22 micron filter. This assay buffer can be stored at room temperature for up to 3 months.
(ii) Preparation of 0.1 N NaOH: An amount of 2 mL of 2.5 N NaOH is added into 48 mL DI water. This solution can be stored at room temperature for up to 3 months.
(iii) Preparation of 4 mg/mL tripeptide GPA stock: An amount of 400 mg (±1 mg) of GPA tripeptide is dissolved into 10 mL 0.1 N NaOH and vortexed until totally dissolved. A sufficient quantity of assay buffer is added to make the volume 100 mL and the solution is dispensed into 0.5 mL aliquots and stored at −70° C. The 4 mg/mL tripeptide GPA stock can be stored at −70° C. for up to one year.
(iv) Preparation of 0.02 N acetic acid: An amount of 1 mL of 1.0 N acetic acid is added to 40 mL of purified water. A sufficient amount of purified water is added to adjust the volume to 50 mL. This solution can be stored at room temperature for up to 1 year.
(v) Preparation of 2 mg/mL SRC substrate stock solution: An amount of 23.3 mL 0.02 N acetic acid is added directly to the vial in which substrate is supplied (supplied in one non-limiting example as 100 mg SRC at 3.75 mg/mL). Other concentrations of SRC substrate may be used. The calculation is below:

100 mg÷3.75 mg/mL=26.7 mL;

Total vol (mL)=(3.75 mg/mL×26.7 mL)/2 mg/mL;

Total vol (50.0 mL)−26.7 mL=23.3 mL

The solutions are mixed thoroughly by inversion and can be stored at 2-8° C. for up to 3 months.

(vi) Preparation of 0.6 mg/mL SRC substrate working solution: An amount of 4.2 mL of assay buffer is added to a 15 mL conical tube. Then, 1.8 mL of 2 mg/mL SRC substrate stock solution is added and the solution is mixed by inversion. This solution should be prepared immediately before addition to plate.
(vii) Preparation of 120 mM Boric Acid pH 9.0: An amount of 7.4 g (±0.5 g) of the boric acid is dissolved in 800 mL DI water. The solution is titrated with NaOH to pH 9.0 and sufficient DI water is added to adjust the volume to 1 L. This solution can be stored at room temperature for up to 3 months.
(viii) Preparation of 1 mM Fluorescamine in Acetone: An amount of 28±2 mg Fluorescamine is dissolved in 100 mL acetone. This solution needed to be freshly prepared and protected from light and moisture.

Preparation of Tripeptide GPA Standard and Serial Dilution

A 0.08 mg/mL (329 μM) tripeptide GPA standard is prepared by making a 50-fold dilution of the 4 mg/mL tripeptide GPA stock in assay buffer (for example, 20 μL 4 mg/mL GPA in 980 μL assay buffer). In the assay plate, row A, 200 μL of 329 μM tripeptide GPA standard is pipetted into A1 and A7. An amount of 100 μL assay buffer is pipetted into A2-A6 and A8-A12.

For the tripeptide GPA standard serial dilution, an amount of 100 μL is transferred from A1 into A2, mixed, an amount of 100 μL is transferred from A2 into A3, and repeated until A5. An amount of 100 μL is taken out from A5 well so that its final volume is 100 μL. The A6 well contains buffer only.

For the second tripeptide GPA standard serial dilution, an amount of 100 μL is transferred from A7 into A8, mixed, an amount of 100 μL is transferred from A8 into A9, and repeated until A11. An amount of 100 μL is taken out from A11 well so that its final volume is 100 μL. The A12 well contains buffer only.

Preparation of Collagenase Test Samples

For collagenase samples (e.g., a lyophilized collagenase drug product), the sample is allowed to come to room temperature for at least 10 minutes and reconstituted to form a 3.0 μg/mL stock solution. Different concentrations may be used. A test collagenase sample (T1A) is prepared from the stock solution by diluting with assay buffer. The procedure is repeated to prepare triplicate test samples (T1A, T1B, T1C).

Discussion

In this method, 50 μL of increasing concentrations of the test collagenase samples are mixed with 50 μL of excess substrate (0.2 mg/mL final concentration) in a 96-well plate. An amount of 50 μL of assay buffer is added to rows C-G in a U-bottomed, 96 well polypropylene reaction plate. 150 μL of collagenase samples are pipetted into row B. Then, a 1/1.5 serial dilution is performed using a multi-channel pipette, by transferring 100 μL of collagenase sample from row B into row C, mixing and repeating the process until row G is reached. An amount of 100 μL is removed and discarded from row G. The Blank is prepared in row H by pipetting 50 μL assay buffer to row H. This row contains no enzyme. Table 12 contains the final collagenase concentrations after adding 50 μL substrate to row B through row H.

TABLE 12
Assay Target Concentrations After Substrate Addition

| | | Collagenase |
|---|---|---|
| Row | Dilution | (ng/ml) |
| A | N/A | N/A |
| B | Stock | 1500 |
| C | 1/1.5 | 100 |
| D | 1/2.3 | 667 |
| E | 1/3.4 | 444 |
| F | 1/5.1 | 296 |
| G | 1/7.6 | 198 |
| H-Blank | N/A | 0 |

Collagenase Reaction

The incubator and temperature probe are turned on (temperature 22±1° C. prior to the addition of substrate to the plates). An amount of 50 μL 0.6 mg·mL SRC substrate is added to each well from row B to row H, added column by column then mixed. The reaction start time begins after the substrate is added to the first column. The plate is covered and placed in the 22±1° C. incubator for a total reaction time of 45±5 minutes. To quench the reaction, 100 μL of 0.5 N HCl is added into each well of the dilution plate, column by column, and mixed. Reaction time ends after the HCl is added to the first column.

Detection

An amount of 195 μL of 120 mM Borate pH 9.0 is added to each well of a Microplate Greiner polypropylene black reading plate. 30 μL of the quenched reaction mixture is transferred from the reaction plate into the corresponding wells of the reading plate and mixed well. Then, 75 μL of 1 mM Fluorescamine is added to each well of the reading plate (using a polypropylene tray to dispense Fluorescamine/acetone) and mixed immediately after every addition. The plate is read within 15 minutes after Fluorescamine addition with a Molecular Devices M2 fluorescence plate reader using the following settings: Excitation 380 nm, Emission 473 nm, cutoff 455 nm, 6 reads/well, PMT medium.

The concentration of GPA (μM) versus the emission at 473 nm and the concentration of collagenase (ng/mL) versus the emission at 473 nm are plotted. For each plot, a linear regression is fitted with no fixed parameters. For collagenase samples, the zero point data are excluded from the linear fit and the entire triplicate data set for each sample is used to generate the plot. The slopes for the tripeptide GPA standard and collagenase samples are determined.

Specific Activity and Relative Potency Determination

The collagenase sample specific activity can by calculated as follows:

SRC Microplate Assay Units=((Slope of Collagenase Sample)/(Slope of Tripeptide GPA×incubation time))×10⁶

The specific activity of the collagenase test sample is determined from the slope of the tripeptide GPA standard and calculated by the curve-fitting program. Using the microplate method, different concentrations of substrate and different times may be used to calculate enzyme kinetics according to Michaelis-Menton.

iii. Collagenase Potency as Measured by SRC Assays

The collagenases useful in the present disclosure may have a potency of about 500 to about 15,000 SRC units/mg. In certain embodiments, the potency is about 500 to about 12,500 SRC units/mg, or about 700 to about 10,000 SRC units/mg, or about 1,000 to about 7,500 SRC units/mg, or 1,500 to about 6,000 SRC units/mg, or about 2,500 to about 5,000 SRC units/mg. Alternatively, the potency may be about 5,000 to about 35,000 f-SRC units/mg, or about 10,000 to about 30,000 f-SRC units/mg, or about 13,000 to about 23,000 f-SRC units/mg, or about 15,000 to about 25,000 f-SRC units/mg. The collagenases may also have a potency of about 980 to 3,510, or about 1,400 to 2,700 SRC Microplate Assay Units.

c. Collagenase Potency in BTC Unit Assay

The Bovine Tendon Collagen Assay for Collagenase is based on the procedure of Mandl et al. (1958), as modified by Keller and Mandl (1963). Since bovine tendon collagen is an insoluble substrate, it is important that it be finely divided. Trypsin is run as a control in order to account for the presence of denatured collagen or other protein impurities. The assay is run in the presence of calcium ions, which are required for collagenase activity. The number of peptides solubilized is determined by reacting the N-terminal amino group of the peptides with ninhydrin and measuring colorimetrically the amount of adjunct formed (Rosen 1957).

The purpose of this procedure is to test the specific activity of collagenase enzyme using a collagen substrate.

Reagents and Solutions

1. Collagen Substrate (collagen)

2. Deionized Water (water)

3. Tris Assay Buffer

4. Trypsin Stock Solution

5. 0.5 M HCl

6. Leucine Standard Assay Solution (1 mM leucine)

7. Rosen Buffer

8. 3% Ninhydrin

9. 50% Isopropanol

Incubation

Set up and label reaction tubes as follows: three tubes for the trypsin controls, six tubes for the Reference Solution and six tubes for each sample under test. Label and uncap each tube. Weigh out 10±1 mg collagen in the order of Table 13 and place the weighed collagen into each reaction tube.

TABLE 13
Order of Weighing and Reaction Tube Numbers

| Order of Weighing | Reaction Tube # |
|---|---|
| 1 | 1 |
| 2 | 4 |
| 3 | 6 |
| 4 | 8 |
| 5 | 10 |
| 6 | 12 |
| 7 | 14 |
| 8 | 16 |
| 9 | 18 |
| 10 | 20 |
| 11 | 2 |
| 12 | 3 |
| 13 | 5 |
| 14 | 7 |
| 15 | 9 |
| 16 | 11 |
| 17 | 13 |
| 18 | 15 |
| 19 | 17 |
| 20 | 19 |
| 21 | 21 |

For samples under test the amount of enzyme should contain an activity between 1.6 to 5.7 nmol 1 eu eq/min per reaction tube (ACT). Undissolved samples should first be dissolved in Tris assay buffer before they are used in the assay. The concentration (before adding to the reaction tubes) should be no less than 0.0065 mg/mL.

Set the reaction tubes according to Table 14 to have a matrix pattern. The following table assumes 2 under test samples. If more or less samples are run, adjust the number of reaction tubes, but retain the pattern. Where volumes are constant, they are listed in Table 14.

TABLE 14
The Matrix Pattern

| | | | S3 | | | |
|---|---|---|---|---|---|---|
| Reaction | S1 | S4 | Standard | | | |
| Tube # | Tris Buffer | Trypsin | Solution | | Samples*1 | Samples*2 |
| 1 | 1960 μL | 40 μL | | | | |
| 2 | 1960 μL | 40 μL | | | | |
| 3 | 1960 μL | 40 μL | | | | |
| 4 | 1940 μL | | 60 | μL | | |
| 5 | 1940 μL | | 60 | μL | | |
| 6 | 1920 μL | | 80 | μL | | |
| 7 | 1920 μL | | 80 | μL | | |
| 8 | 1900 μL | | 100 | μL | | |
| 9 | 1900 μL | | 100 | μL | | |
| 10 | 1970 μL | | | | 30 μL | |
| 11 | 1970 μL | | | | 30 μL | |
| 12 | 1960 μL | | | | 40 μL | |
| 13 | 1960 μL | | | | 40 μL | |
| 14 | 1950 μL | | | | 50 μL | |
| 15 | 1950 μL | | | | 50 μL | |
| 16 | 1970 μL | | | | | 30 μL |
| 17 | 1970 μL | | | | | 30 μL |
| 18 | 1960 μL | | | | | 40 μL |
| 19 | 1960 μL | | | | | 40 μL |
| 20 | 1950 μL | | | | | 50 μL |
| 21 | 1950 μL | | | | | 50 μL |

*Suggested maximum number of samples is 3.

Cap the reaction tubes. Mix the contents gently but thoroughly. Place the reaction tubes in a 37° C. water bath. Incubate for 22±0.5 hours. Record the actual time incubation started a, 37° C., the number of the water bath used, the lot number of the collagen Lipid, and the collagen correction factor for the lot used and the lot numbers of all solutions used.

Quenching And Filtration

Label a filtrate tube to correspond to each reaction tube incubated. Place a funnel containing and folded filter paper onto each labeled filtrate tube. At the end of the incubation period, remove the reaction tubes from the water bath. Record the actual time incubation ended.

Uncap the reaction tubes and discard the caps. Quench the reaction by dispensing 2 mL 0.5 M HCl into each reaction tube. Mix the contents of the tubes thoroughly. Filter the contents of each reaction tube into the appropriate filtrate tube.

The previous two steps need to be finished as quickly as possible because undigested collagen could be dissolved by HCl in a short time. The filtrate may be stored refrigerated in covered filtrate tubes for up to 95.5 hours before color development. Record the refrigeration and time stored.

Color Development

Set up and label boiling tubes as follows: six tubes for the water and the leucine controls (Step 1) and two tubes for each filtrate tub (Step 1). Place the following amounts of water and leucine standard assay solution into the six leucine control tubes.

| Tube # | 1 | 2 | 3 | 4 | 5 | 6 |
|---|---|---|---|---|---|---|
| Water (μL) | 1000 | 900 | 850 | 800 | 750 | 700 |
| Leu (μL) | 0 | 100 | 150 | 200 | 250 | 300 |
| Leu (nmol) | 0 | 100 | 150 | 200 | 250 | 300 |

Pipette 0.8 mL of water into each boiling tube (Step 2). Pipette 0.2 mL of filtrate from each sample into the appropriately labeled boiling tubes. Dispense 0.5 mL of Rosen buffer into each boiling tube. Under a containment hood, dispense 0.5 mL of 3% ninhydrin into each boiling tube. Mix the contents of each tube thoroughly on a vortex mixer. Place the boiling tubes in a boiling water bath in a fume hood. Boil for 15±1 minutes. At the end of the boiling period, remove the boiling tubes from the water bath. Under a containment hood, dispense 5.0 mL of 50% Isopropanol into each boiling tube and mix the contents thoroughly. Allow the boiling tubes to reach ambient temperature (at least 10 minutes) before reading the absorbances.

Reading of Absorbances

Read the absorbances of the tubes while working under a containment hood. Turn on the spectrophotometer and allow it to warm up. Set the wavelength of the spectrophotometer to 570 nm. Zero the spectrophotometer against 50% Isopropanol. Read the absorbances (A570) of the water, leucine, trypsin controls and the samples under test. Record the time that the first sample is read, in hours. Record the readings as 1000×A570 and record the time that the last sample is read, in hours. All readings are to be done within a 1-hour interval.

Calculations Principles

Calculate, in minutes, the total reading time and the total time of incubation. The total reading time should be less than 60 minutes and the total time of incubation should be between 1290-1350 minutes. Using the linear least square method, calculate the slope “b” and correlation coefficient “r” for leucine standards (x=nmol leucine vs y=A₅₇₀ reading). The unit for “b” value is A₅₇₀/nmol leucine. Record “b” value to two decimal places. b value for leucine should be between 2.88-3.33. Calculate the average reading for the trypsin controls (T). The average reading for the trypsin controls (T) should be 221-338. Record this average to the nearest whole number (Step A). Average duplicated sample A570 reading for each reaction tube. Record this number to the nearest whole number. Subtract average trypsin (Step A) from the average sample A570 reading to get the net sample reading.

Calculate the activity (ACT) per tube, in nmol leu eq/min, as follows:

$$ACT ( nmol lue eq / min ) = \frac{( Net sample reading ) ( 20 )}{( b ) ( Time in minutes )}$$

where 20 is the dilution factor for the amount of reaction mixture developed and “b” is the slope of the leucine standard curve. Record this number to one decimal point. The activity per tube of the samples under test should be 1.6-5.7 nmol leu eq/min.

Calculate the activity in BTC units, as follows:

BTC units=activity in nmol 1 eu eq/min×collagen correction factor.

Calculate the activity in BTC units/mL of the sample as follows:

$$BTC unit / mL = \frac{Activity in BTC units}{Sample volume used in mL}$$

Calculate the specific activity of the sample in BTC units/mm follows:

$$BTC unit / mg = \frac{Activity in BTC units / mL}{Protein Concentration in mg / mL}$$

The conversion of BTC units to ABC units is:

ABC units=BTC units×1.09

i. BTC Units and ABC Units

Various collagenase compositions may be employed wherein the collagenase has a specific activity of about 5,000 BTC units/mg to about 25,000 BTC units/mg, or about 10,000 BTC units/mg to about 25,000 BTC units/mg, or about 15,000 BTC units/mg, or about 17,500 BTC units/mg, or about 20,000 BTC units/mg, or about 22,500 BTC units/mg, or about 9,175 BTC units/0.58 mg, or 15,817 BTC units/mg wherein “mg” refers to the amount of collagenase(s) present in a composition (as distinct from excipients and other constituents).

Further, various collagenase compositions may be employed wherein the collagenase has a specific activity of about 5,000 ABC units/mg to about 25,000 ABC units/mg, or about 10,000 ABC units/mg to about 25,000 ABC units/mg, or about 15,000 ABC units/mg, or about 17,500 ABC units/mg, or about 20,000 ABC units/mg, or about 22,500 ABC units/mg, or about 10,000 ABC units/0.58 mg, or 17,241 ABC units/mg wherein “mg” refers to the amount of collagenase(s) present in a composition (as distinct from excipients and other constituents).

d. Other Assays

Assay methods utilizing labelled collagen have been reported by Gisslow et al., Anal. Biochem., 68: 70-78 (1975); Robertson et al., Clinica Chimica Acta, 42:43-45 (1972); Sakamoto et al., A New Method for the Assay of Tissue Collagenase (36297) (1972). One other assay is the Worthington Biochemical Corp. Assay (http://www.worthington-biochem.com/CLS/assay.html) (accessed Jul. 3, 2019).

4. Doses of Collagenase

As for collagenase doses employed herein, the present disclosure provides for therapeutically effective amounts of collagenase sufficient to bind and lyse the septae upon subcutaneous injection to result in a decreased appearance of cellulite compared to pretreatment baseline.

In one embodiment, the collagenase may be injected in an amount of about 0.01 mg to about 20 mg in a single or divided doses. In another embodiment, the collagenase may be injected in an amount of about 0.05 mg to about 15 mg in a single or divided doses. In another embodiment, the collagenase may be injected in an amount of about 0.10 mg to about 10 mg in a single or divided doses. In another embodiment, the collagenase may be injected in an amount of about 0.15 mg to about 5 mg in a single or divided doses. In another embodiment, the collagenase may be injected in an amount of about 0.20 mg to about 3 mg in a single or divided doses. In another embodiment, the collagenase may be injected in an amount of about 0.25 mg to about 2 mg in a single or divided doses. In yet another embodiment, the collagenase may be injected in an amount of about 0.05 mg, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, or about 6.00 mg.

In one embodiment, the collagenase may have a Vₘₐₓ of about 2.6 min⁻¹ to 5.2 min⁻¹, as measured using the SRC assay. In another embodiment, the collagenase may have a Vₘₐₓ of about 3.0 min⁻¹ to 5.0 min⁻¹, as measured using the SRC assay. In another embodiment, the collagenase may have a Vₘₐₓ of about 3.4 min⁻¹ to 4.8 min⁻¹, as measured using the SRC assay. In still another embodiment, the collagenase may have a Vₘₐₓ of about 3.5 min⁻¹ to 4.5 min⁻¹, as measured using the SRC assay. In yet another embodiment, the collagenase may have a V. of about 2.0 min⁻¹, about 2.1 min⁻¹, about 2.2 min⁻¹, about 2.3 min⁻¹, about 2.4 min⁻¹, about 2.5 min¹, about 2.6 min⁻¹, about 2.7 min⁻¹, about 2.8 min⁻¹, about 2.9 min⁻¹, about 3.0 min⁻¹, about 3.1 min¹, about 3.2 min⁻¹, about 3.3 min⁻¹, about 3.4 min⁻¹, about 3.5 min⁻¹, about 3.6 min⁻¹, about 3.7 min¹, about 3.8 min⁻¹, about 3.9 min⁻¹, about 4.0 min⁻¹, about 4.1 min⁻¹, about 4.2 min⁻¹, about 4.3 min¹, about 4.4 min⁻¹, about 4.5 min⁻¹, about 4.6 min⁻¹, about 4.7 min⁻¹, about 4.8 min⁻¹, about 4.9 min¹, about 5.0 min⁻¹, about 5.1 min⁻¹, about 5.2 min⁻¹, about 5.3 min⁻¹, about 5.4 min⁻¹, about 5.5 min¹, about 5.6 min⁻¹, about 5.7 min⁻¹, about 5.8 min⁻¹, about 5.9 min⁻¹, or about 6.0 min⁻¹, as measured using the SRC assay. In another embodiment, the collagenase may have a Vₘₐₓ of about 0.7 min⁻¹ to 7.6 min⁻¹, as measured using the SRC assay, or about 1 to 6, or about 2 to 5, or about 3 to 4 min⁻¹, as measured using the SRC assay.

In one embodiment, the collagenase may have a Vₘₐₓ of about 135 min⁻¹ to 268 min⁻¹, as measured using the GPA assay. In another embodiment, the collagenase may have a Vₘₐₓ of about 150 min⁻¹ to 250 min⁻¹, as measured using the GPA assay. In another embodiment, the collagenase may have a Vₘₐₓ of about 175 min⁻¹ to 225 min⁻¹, as measured using the GPA assay. In still another embodiment, the collagenase may have a Vₘₐₓ of about 130 min⁻¹, about 135 min⁻¹, about 140 min⁻¹, about 145 min⁻¹, about 150 min⁻¹, about 155 min⁻¹, about 160 min⁻¹, about 165 min⁻¹, about 170 min⁻¹, about 175 min⁻¹, about 180 min⁻¹, about 185 min⁻¹, about 190 min⁻¹, about 195 min⁻¹, about 200 min⁻¹, about 205 min⁻¹, about 210 min⁻¹, about 215 min⁻¹, about 220 min⁻¹, about 225 min⁻¹, about 230 min⁻¹, about 235 min⁻¹, about 240 min⁻¹, about 245 min⁻¹, about 250 min⁻¹, about 255 min⁻¹, about 260 min⁻¹, about 265 min⁻¹, about 270 min⁻¹, about 275 min⁻¹, or about 280 min⁻¹, as measured using the GPA assay. In another embodiment, the collagenase may have a Vₘₐₓ of about 4 min⁻¹ to 400 min⁻¹, as measured using the GPA assay, or about 0.3 to 30.5, or about 10 to 375, or about 20 to 350, or about 50 to 300, or about 100 to 275 min⁻¹, as measured using the GPA assay.

In one embodiment, the collagenase may have a Kₘ of about 75 mM to 147 mM, as measured using the SRC assay. In another embodiment, the collagenase may have a Kₘ of about 80 mM to 140 mM, as measured using the SRC assay. In another embodiment, the collagenase may have a Kₘ of about 85 mM to 130 mM, as measured using the SRC assay. In another embodiment, the collagenase may have a Kₘ of about 90 mM to 120 mM, as measured using the SRC assay. In yet another embodiment, the collagenase may have a Kₘ of about 70 mM, about 72 mM, about 75 mM, about 77 mM, about 80 mM, about 82 mM, about 85 mM, about 87 mM, about 90 mM, about 92 mM, about 95 mM, about 97 mM, about 100 mM, about 102 mM, about 105 mM, about 107 mM, about 110 mM, about 112 mM, about 115 mM, about 117 mM, about 120 mM, about 122 mM, about 125 mM, about 127 mM, about 130 mM, about 132 mM, about 135 mM, about 137 mM, about 140 mM, about 142 mM, about 145 mM, about 147 mM, about 150 mM, about 152 mM, about 155 mM, or about 157 mM, as measured using the SRC assay. In another embodiment, the collagenase may have a Kₘ of about 4.4 mM to 437 mM, as measured using the SRC assay, or about 5 to 400, or about 20 to 375, or about 50 to 325, or about 100 to 275, or about 150 to 250 mM, or about 4.1 to 410 nanoMolar as measured using the SRC assay.

In one embodiment, the collagenase may have a Kₘ of about 0.03 mM to 3.1 mM, as measured using the GPA assay. In another embodiment, the collagenase may have a K. of about 1.00 mM to 1.60 mM, as measured using the GPA assay. In another embodiment, the collagenase may have a Kₘ of about 1.10 mM to 1.50 mM, as measured using the GPA assay. In another embodiment, the collagenase may have a Kₘ of about 1.15 mM to 1.40 mM, as measured using the GPA assay. In yet another embodiment, the collagenase may have a Kₘ of about 0.80 mM, about 0.82 mM, about 0.85 mM, about 0.87 mM, about 0.90 mM, about 0.92 mM, about 0.95 mM, about 0.97 mM, about 1.00 mM, about 1.02 mM, about 1.05 mM, about 1.07 mM, about 1.10 mM, about 1.12 mM, about 1.15 mM, about 1.17 mM, about 1.20 mM, about 1.22 mM, about 1.25 mM, about 1.27 mM, about 1.30 mM, about 1.32 mM, about 1.35 mM, about 1.37 mM, about 1.40 mM, about 1.42 mM, about 1.45 mM, about 1.47 mM, about 1.50 mM, about 1.52 mM, about 1.55 mM, about 1.57 mM, about 1.60 mM, about 1.62 mM, about 1.65 mM, or about 1.67 mM, as measured using the GPA assay. In another embodiment, the collagenase may have a Kₘ of about 0.027 mM to 2.7 mM, as measured using the GPA assay, or about 0.1 to 2, or about 0.5 to 1.5, or about 1 to 1.35 mM, as measured using the GPA assay.

In one embodiment, the collagenase may have a K_{cat} of about 36 sec⁻¹ to 671 sec⁻¹, as measured using the SRC assay. In another embodiment, the collagenase may have a K_{cat} of about 50 sec⁻¹ to 600 sec⁻¹, as measured using the SRC assay. In another embodiment, the collagenase may have a K_{cat} of about 60 sec⁻¹ to 500 sec⁻¹, as measured using the SRC assay. In another embodiment, the collagenase may have a K_{cat} of about 70 sec⁻¹ to 400 sec⁻¹, as measured using the SRC assay. In still another embodiment, the collagenase may have a K_{cat} of about 100 sec⁻¹ to 350 sec⁻¹, as measured using the SRC assay. In another embodiment, the collagenase may have a Kcat of about 30 sec⁻¹, about 40 sec⁻¹, about 50 sec⁻¹, about 60 sec⁻¹, about 70 sec⁻¹, about 80 sec⁻¹, about 90 sec⁻¹, about 100 sec⁻¹, about 110 sec⁻¹, about 120 sec⁻¹, about 130 sec⁻¹, about 140 sec⁻¹, about 150 sec⁻¹, about 160 sec⁻¹, about 170 sec⁻¹, about 180 sec⁻¹, about 190 sec⁻¹, about 200 sec⁻¹, about 210 sec⁻¹, about 220 sec⁻¹, about 230 sec⁻¹, about 240 sec⁻¹, about 250 sec⁻¹, about 260 sec⁻¹, about 270 sec⁻¹, about 280 sec⁻¹, about 290 sec⁻¹, about 300 sec⁻¹, about 310 sec⁻¹, about 320 sec⁻¹, about 330 sec⁻¹, about 340 sec⁻¹, about 350 sec⁻¹, about 360 sec⁻¹, about 370 sec⁻¹, about 380 sec⁻¹, about 390 sec⁻¹, about 400 sec⁻¹, about 410 sec⁻¹, about 420 sec⁻¹, about 430 sec⁻¹, about 440 sec⁻¹, about 450 sec⁻¹, about 460 sec⁻¹, about 470 sec⁻¹, about 480 sec⁻¹, about 490 sec⁻¹, about 500 sec⁻¹, about 510 sec⁻¹, about 520 sec⁻¹, about 530 sec⁻¹, about 540 sec⁻¹, about 550 sec⁻¹, about 560 sec⁻¹, about 570 sec⁻¹, about 580 sec⁻¹, about 590 sec⁻¹, about 600 sec⁻¹, about 610 sec⁻¹, about 620 sec⁻¹, about 630 sec⁻¹, about 640 sec⁻¹, about 650 sec⁻¹, about 660 sec⁻¹, about 670 sec⁻¹, about 680 sec⁻¹, about 690 sec⁻¹, about 700 sec⁻¹, about 710 sec⁻¹, about 720 sec⁻¹, about 730 sec⁻¹, about 740 sec⁻¹, about 750 sec⁻¹, or about 760 sec⁻¹, as measured using the SRC assay. In another embodiment, the collagenase may have a K_{cat} of about 1 sec⁻¹ to 107 sec⁻¹, as measured using the SRC assay, or about 10 to 100, or about 20 to 80, or about 30 to 70, or about 40 to 60 sec⁻¹, as measured using the SRC assay.

In one embodiment, the collagenase may have a K_{cat} of about 90 to 10,000, or about 41,000 sec⁻¹ to about 81,000 sec⁻¹, as measured using the GPA assay. In another embodiment, the collagenase may have a K_{cat} of about 45,000 sec⁻¹ to about 75,000 sec⁻¹, as measured using the GPA assay. In another embodiment, the collagenase may have a K_{cat} of about 50,000 sec⁻¹ to about 70,000 sec⁻¹, as measured using the GPA assay. In another embodiment, the collagenase may have a K_{cat} of about 55,000 sec⁻¹ to about 65,000 sec⁻¹, as measured using the GPA assay. In still another embodiment, the collagenase may have a K_{cat} of about 35,000 sec⁻¹, about 37,500 sec⁻¹, about 40,000 sec⁻¹, about 42,500 sec⁻¹, about 45,000 sec⁻¹, about 47,500 sec⁻¹, about 50,000 sec⁻¹, about 52,500 sec⁻¹, about 55,000 sec⁻¹, about 57,500 sec⁻¹, about 60,000 sec⁻¹, about 62,500 sec⁻¹, about 65,000 sec⁻¹, about 67,500 sec⁻¹, about 70,000 sec⁻¹, about 72,500 sec⁻¹, about 75,000 sec⁻¹, about 77,500 sec⁻¹, about 80,000 sec⁻¹, about 82,500 sec⁻¹, or about 85,000 sec⁻¹, as measured using the GPA assay. In another embodiment, the collagenase may have a K_{cat} of about 1215 sec⁻¹ to about 120,000 sec⁻¹, as measured using the GPA assay, or about 2,000 to 100,000, or about 10,000 to 90,000, or about 20,000 to 80,000, or about 30,000 to 70,000, or about 40,000 to 60,000 sec⁻¹, as measured using the GPA assay.

In one embodiment, the collagenase may have 1/K_{cat} of about 376 to 38,000 μsec, or about 14,000 μsec to about 28,000 μsec, as measured using the SRC assay. In another embodiment, the collagenase may have 1/K_{cat} of about 16,000 μsec to about 26,000 μsec, as measured using the SRC assay. In one embodiment, the collagenase may have 1/K_{cat} of about 18,000 μsec to about 24,000 μsec, as measured using the SRC assay. In one embodiment, the collagenase may have 1/K_{cat} of about 20,000 μsec to about 22,000 μsec, as measured using the SRC assay. In still another embodiment, the collagenase may have 1/K_{cat} of about 12,500 μsec, about 12,750 μsec, about 13,000 μsec, about 13,250 μsec, about 13,500 μsec, about 13,750 μsec, about 14,000 μsec, about 14,250 μsec, about 14,750 μsec, about 15,000 μsec, about 15,250 μsec, about 15,500 μsec, about 15,750 μsec, about 16,000 μsec, about 16,250 μsec, about 16,500 μsec, about 16,750 μsec, about 17,000 μsec, about 17,250 μsec, about 17,500 μsec, about 17,750 μsec, about 18,000 μsec, about 18,250 μsec, about 18,500 μsec, about 18,750 μsec, about 19,000 μsec, about 19,250 μsec, about 19,500 μsec, about 19,750 μsec, about 20,000 μsec, about 20,250 μsec, about 20,500 μsec, about 20,750 μsec, about 21,000 μsec, about 21,250 μsec, about 21,500 μsec, about 21,750 μsec, about 22,000 μsec, about 22,250 μsec, about 22,500 μsec, about 22,750 μsec, about 23,000 μsec, about 23,250 μsec, about 23,500 μsec, about 23,750 μsec, about 24,000 μsec, about 24,250 μsec, about 24,500 μsec, about 24,750 μsec, about 25,000 μsec, about 25,250 μsec, about 25,500 μsec, about 25,750 μsec, about 26,000 μsec, about 26,250 μsec, about 26,500 μsec, about 26,750 μsec, about 27,000 μsec, about 27,250 μsec, about 27,500 μsec, about 27,750 μsec, about 28,000 μsec, about 28,250 μsec, about 28,500 μsec, about 28,750 μsec, about 29,000 μsec, or about 29,250 μsec, as measured using the SRC assay. In another embodiment, the collagenase may have 1/K_{cat} of about 370 μsec to about 36,700 μsec, as measured using the SRC assay, or about 750 to 30,000, or about 2,500 to 25,000, or about 5,000 to 20,000, or about 10,000 to 18,000, or about 15,000 μsec, as measured using the SRC assay.

In one embodiment, the collagenase may have 1/K_{cat} of about 4 μsec to about 430 μsec, as measured using the GPA assay. In another embodiment, the collagenase may have 1/K_{cat} of about 14 μsec to about 23 μsec, as measured using the GPA assay. In another embodiment, the collagenase may have 1/K_{cat} of about 16 μsec to about 21 μsec, as measured using the GPA assay. In still another embodiment, the collagenase may have 1/K_{cat} of about 10.0 μsec, about 10.2 μsec, about 10.4 μsec, about 10.6 μsec, about 10.8 μsec, about 11.0 μsec, about 11.2 μsec, about 11.4 μsec, about 11.6 μsec, about 11.8 μsec, about 12.0 μsec, about 12.2 μsec, about 12.4 μsec, about 12.6 μsec, about 12.8 μsec, about 13.0 μsec, about 13.2 μsec, about 13.4 μsec, about 13.6 μsec, about 13.8 μsec, about 14.0 μsec, about 14.2 μsec, about 14.4 μsec, about 14.6 μsec, about 14.8 μsec, about 15.0 μsec, about 15.2 μsec, about 15.4 μsec, about 15.6 μsec, about 15.8 μsec, about 16.0 μsec, about 16.2 μsec, about 16.4 μsec, about 16.6 μsec, about 16.8 μsec, about 17.0 μsec, about 17.2 μsec, about 17.4 μsec, about 17.6 μsec, about 17.8 μsec, about 18.0 μsec, about 18.2 μsec, about 18.4 μsec, about 18.6 μsec, about 18.8 μsec, about 19.0 μsec, about 19.2 μsec, about 19.4 μsec, about 19.6 μsec, about 19.8 μsec, about 20.0 μsec, about 20.2 μsec, about 20.4 μsec, about 20.6 μsec, about 20.8 μsec, about 21.0 μsec, about 21.2 μsec, about 21.4 μsec, about 21.6 μsec, about 21.8 μsec, about 22.0 μsec, about 22.2 μsec, about 22.4 μsec, about 22.6 μsec, about 22.8 μsec, about 23.0 μsec, about 23.2 μsec, about 23.4 μsec, about 23.6 μsec, about 23.8 μsec, about 24.0 μsec, about 24.2 μsec, about 24.4 μsec, about 24.6 μsec, about 24.8 μsec, about 25.0 μsec, about 25.2 μsec, about 25.4 μsec, about 25.6 μsec, about 25.8 μsec, about 26.0 μsec, about 26.2 μsec, about 26.4 μsec, about 26.8 μsec, about 27.0 μsec, about 27.2 μsec, or about 27.4 μsec, as measured using the GPA assay. In another embodiment, the collagenase may have 1/K_{cat} of about 0.3 μsec to about 32 μsec, as measured using the GPA assay, or about 1 to 30, or about 5 to 25, or about 10 to 20, or about 15 μsec, as measured using the GPA assay.

In one embodiment, the collagenase may have a K_{cat}/Kₘ of about 5,140 mM⁻¹sec⁻¹ to about 508,814 mM⁻¹sec⁻¹, as measured using the SRC assay. In another embodiment, the collagenase may have a K_{cat}/Kₘ of about 0.50 mM⁻¹sec⁻¹ to about 7.75 mM⁻¹sec⁻¹, as measured using the SRC assay. In another embodiment, the collagenase may have a K_{cat}/Kₘ of about 0.75 mM⁻¹sec⁻¹ to about 7.00 mM⁻¹sec⁻¹, as measured using the SRC assay. In still another embodiment, the collagenase may have a K_{cat}/Kₘ of about 1.00 mM⁻¹sec⁻¹ to about 6.00 mM⁻¹sec⁻¹, as measured using the SRC assay. In still another embodiment, the collagenase may have a K_{cat}/Kₘ of about 0.10 mM⁻¹sec⁻¹, about 0.20 mM⁻¹sec⁻¹, about 0.30 mM⁻¹sec⁻¹, about 0.40 mM⁻¹sec⁻¹, about 0.50 mM⁻¹sec⁻¹, about 0.60 mM⁻¹sec⁻¹, about 0.70 mM⁻¹sec⁻¹, about 0.80 mM⁻¹sec⁻¹, about 0.90 mM⁻¹sec⁻¹, about 1.00 mM⁻¹sec⁻¹, about 1.10 mM⁻¹sec⁻¹, about 1.20 mM⁻¹sec¹, about 1.30 mM⁻¹sec¹, about 1.40 mM⁻¹sec⁻¹, about 1.50 mM⁻¹sec⁻¹, about 1.60 mM⁻¹sec⁻¹, about 1.70 mM⁻¹sec⁻¹, about 1.80 mM⁻¹sec⁻¹, about 1.90 mM⁻¹sec⁻¹, about 2.00 mM⁻¹sec⁻¹, about 2.10 mM⁻¹sec⁻¹, about 2.20 mM⁻¹sec⁻¹, about 2.30 mM⁻¹sec⁻¹, about 2.40 mM⁻¹sec⁻¹, about 2.50 mM⁻¹sec⁻¹, about 2.60 mM⁻¹sec⁻¹, about 2.70 mM⁻¹sec⁻¹, about 2.80 mM⁻¹sec⁻¹, about 2.90 mM⁻¹sec⁻¹, about 3.00 mM⁻¹sec¹, about 3.10 mM⁻¹sec⁻¹, about 3.20 mM⁻¹sec⁻¹, about 3.30 mM⁻¹sec⁻¹, about 3.40 mM⁻¹sec⁻¹, about 3.50 mM⁻¹sec⁻¹, about 3.60 mM⁻¹sec⁻¹, about 3.70 mM⁻¹sec⁻¹, about 3.80 mM⁻¹sec⁻¹, about 3.90 mM⁻¹sec⁻¹, about 4.00 mM⁻¹sec⁻¹, about 4.10 mM⁻¹sec⁻¹, about 4.20 mM⁻¹sec⁻¹, about 4.30 mM⁻¹sec⁻¹, about 4.40 mM⁻¹sec⁻¹, about 4.50 mM⁻¹sec⁻¹, about 4.60 mM⁻¹sec¹, about 4.70 mM⁻¹sec¹, about 4.80 mM⁻¹sec⁻¹, about 4.90 mM⁻¹sec⁻¹, about 5.00 mM⁻¹sec⁻¹, about 5.10 mM⁻¹sec⁻¹, about 5.20 mM⁻¹sec⁻¹, about 5.30 mM⁻¹sec⁻¹, about 5.40 mM⁻¹sec⁻¹, about 5.50 mM⁻¹sec⁻¹, about 5.60 mM⁻¹sec⁻¹, about 5.70 mM⁻¹sec⁻¹, about 5.80 mM⁻¹sec⁻¹, about 5.90 mM⁻¹sec⁻¹, about 6.00 mM⁻¹sec⁻¹, about 6.10 mM⁻¹sec⁻¹, about 6.20 mM⁻¹sec⁻¹, about 6.30 mM⁻¹sec⁻¹, about 6.40 mM⁻¹sec¹, about 6.50 mM⁻¹sec⁻¹, about 6.60 mM⁻¹sec⁻¹, about 6.70 mM⁻¹sec⁻¹, about 6.80 mM⁻¹sec⁻¹, about 6.90 mM⁻¹sec⁻¹, about 7.00 mM⁻¹sec⁻¹, about 7.10 mM⁻¹sec⁻¹, about 7.20 mM⁻¹sec⁻¹, about 7.30 mM⁻¹sec⁻¹, or about 7.40 mM⁻¹sec⁻¹, as measured using the SRC assay. In another embodiment, the collagenase may have a K_{cat}/Kₘ of about 0.0048 mM⁻¹sec⁻¹ to about 0.47 mM⁻¹sec⁻¹, as measured using the SRC assay, or about 0.009 to about 0.3, or about 0.01 to about 0.25, or about 0.1 to 0.25 mM⁻¹sec⁻¹, as measured using the SRC assay.

In one embodiment, the collagenase may have a Kₑₐₜ/Kₘ of about 60 mM⁻¹sec⁻¹ to about 6,000 mM⁻¹sec⁻¹, as measured using the GPA assay. In another embodiment, the collagenase may have a K_{cat}/Kₘ of about 30,000 mM⁻¹sec⁻¹ to about 85,000 mM⁻¹sec⁻¹, as measured using the GPA assay. In another embodiment, the collagenase may have a K_{cat}/Kₘ of about 36,000 mM⁻¹sec⁻¹ to about 77,000 mM⁻¹sec⁻¹, as measured using the GPA assay. In yet another embodiment, the collagenase may have a K_{cat}/Kₘ of about 40,000 mM⁻¹sec⁻¹ to about 70,000 mM⁻¹sec⁻¹, as measured using the GPA assay. In still another embodiment, the collagenase may have a Kat/Kₘ of about 40,000 mM⁻¹sec⁻¹, about 42,000 mM⁻¹sec⁻¹, about 44,000 mM⁻¹sec⁻¹, about 46,000 mM⁻¹sec⁻¹, about 48,000 mM⁻¹sec⁻¹, about 50,000 mM⁻¹sec⁻¹, about 52,000 mM⁻¹sec¹, about 54,000 mM⁻¹sec⁻¹, about 56,000 mM⁻¹sec⁻¹, about 58,000 mM⁻¹sec⁻¹, about 60,000 mM⁻¹sec⁻¹, about 62,000 mM⁻¹sec⁻¹, about 64,000 mM⁻¹sec⁻¹, about 66,000 mM⁻¹sec⁻¹, about 68,000 mM⁻¹sec⁻¹, about 70,000 mM⁻¹sec⁻¹, about 72,000 mM⁻¹sec⁻¹, about 74,000 mM⁻¹sec⁻¹, about 76,000 mM⁻¹sec⁻¹, about 78,000 mM⁻¹sec⁻¹, about 80,000 mM⁻¹sec⁻¹, about 82,000 mM⁻¹sec⁻¹, about 84,000 mM⁻¹sec⁻¹, about 86,000 mM⁻¹sec⁻¹, about 88,000 mM⁻¹sec⁻¹, about 90,000 mM⁻¹sec⁻¹, about 92,000 mM⁻¹sec⁻¹, about 94,000 mM⁻¹sec⁻¹, or about 96,000 mM⁻¹sec⁻¹, as measured using the GPA assay. In another embodiment, the collagenase may have a K_{cat}/Kₘ of about 900 mM⁻¹sec⁻¹ to about 90,000 mM⁻¹sec⁻¹, as measured using the GPA assay, or about 2,000 to 80,000, or about 10,000 to 70,000, or about 20,000 to 60,000, or about 30,000 to 50,000, or about 40,000 to 45,000 mM⁻¹sec⁻¹, as measured using the GPA assay.

In one embodiment, the collagenase may have a molecular mass of about 60 kDa to about 130 kDa. In another embodiment, the collagenase may have a molecular mass of about 70 kDa to about 130 kDa. In another embodiment, the collagenase may have a molecular mass of about 80 kDa to about 120 kDa. In still another embodiment, the collagenase may have a molecular mass of about 90 kDa to about 120 kDa. In another embodiment, the collagenase may have a molecular mass of about 100 kDa to about 110 kDa. In yet another embodiment, the collagenase may have a molecular mass of about 55 kDa, about 57 kDa, about 60 kDa, about 62 kDa, about 65 kDa, about 67 kDa, about 70 kDa, about 72 kDa, about 75 kDa, about 77 kDa, about 80 kDa, about 82 kDa, about 85 kDa, about 87 kDa, about 90 kDa, about 92 kDa, about 95 kDa, about 97 kDa, about 100 kDa, about 102 kDa, about 105 kDa, about 107 kDa, about 110 kDa, about 112 kDa, about 115 kDa, about 117 kDa, about 120 kDa, about 122 kDa, about 125 kDa, about 127 kDa, about 130 kDa, about 132 kDa, about 135 kDa, or about 137 kDa.

In one embodiment, the collagenase may have a purity of at least 80%, as measured by reverse phase HPLC. In another embodiment, the collagenase may have a purity og about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, as measured by reverse phase HPLC. In still another embodiment, the collagenase may comprise less than or equal to 1% by area of clostripain. In another embodiment, the collagenase may comprise less than or equal to 1% by area of gelatinase. In another embodiment, the collagenase may comprise less than or equal to 1% by area of leupeptin. In still another embodiment, the collagenase may comprise less than or equal to 1 cfu/mL bioburden.

In one embodiment, the collagenase may comprise a potency (i.e., specific activity) of about 500 to about 30,000 SRC units/mg. In another embodiment, the collagenase may comprise a potency of about 2,500 to about 25,000 SRC units/mg. In another embodiment, the collagenase may comprise a potency of about 5,000 to about 20,000 SRC units/mg. In still another embodiment, the collagenase may comprise a potency of about 500, about 1,000, about 1,500, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, about 25,000, about 25,500, about 26,000, about 26,500, about 27,000, about 27,500, about 28,000, about 28,500, about 29,000, about 29,500, or about 30,000 SRC units/mg.

In one embodiment, the collagenase may comprise a potency (i.e., specific activity) of about 5,000 to about 30,000 f-SRC units/mg. In another embodiment, the collagenase may comprise a potency of about 7,500 to about 25,000 f-SRC units/mg. In another embodiment, the collagenase may comprise a potency of about 10,000 to about 20,000 f-SRC units/mg. In still another embodiment, the collagenase may comprise a potency of about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, about 25,000, about 25,500, about 26,000, about 26,500, about 27,000, about 27,500, about 28,000, about 28,500, about 29,000, about 29,500, or about 30,000 f-SRC units/mg.

In one embodiment, the collagenase may comprise a potency of about 100,000 to about 400,000 GPA units/mg. In another embodiment, the collagenase may comprise a potency of about 150,000 to about 350,000 GPA units/mg. In another embodiment, the collagenase may comprise a potency of about 200,000 to about 300,000 GPA units/mg. Instill another embodiment, the collagenase may comprise a potency of about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about 300,000, about 310,000, about 320,000, about 330,000, about 340,000, about 350,000, about 360,000, about 370,000, about 380,000, about 390,000, or about 400,000 GPA units/mg.

In one embodiment, the collagenase may comprise a potency of about 175,000 to about 500,000 f-GPA units/mg. In another embodiment, the collagenase may comprise a potency of about 250,000 to about 450,000 f-GPA units/mg. In another embodiment, the collagenase may comprise a potency of about 300,000 to about 400,000 GPA units/mg. In still another embodiment, the collagenase may comprise a potency of about 175,000, about 185,000, about 195,000, about 205,000, about 215,000, about 225,000, about 235,000, about 245,000, about 255,000, about 265,000, about 275,000, about 285,000, about 295,000, about 305,000, about 315,000, about 325,000, about 335,000, about 345,000, about 355,000, about 365,000, about 375,000, about 385,000, about 395,000, about 405,000, about 415,000, about 425,000, about 435,000, about 445,000, about 455,000, about 465,000, about 475,000, about 485,000, or about 495,000 f-GPA units/mg.

In one embodiment, the collagenase may comprise a potency of about 5,000 to about 25,000 ABC units/mg. In one embodiment, the collagenase may comprise a potency of about 7,500 to about 20,000 ABC units/mg. In one embodiment, the collagenase may comprise a potency of about 10,000 to about 17,500 ABC units/mg. In another embodiment, the collagenase may comprise about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, or about 25,000 ABC units/mg.

In some embodiments, the collagenase present in the composition comprises collagenase I and collagenase II in a ratio of approximately 1:1. Other ratios of collagenase I and collagenase II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of collagenase I and collagenase II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In another embodiment, the collagenase composition comprises CCH having an AUX I and AUX II ratio of approximately 1:1. Other ratios of AUX I and AUX II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of AUX I and AUX II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In other examples, the collagenase composition may be a liquid or is reconstituted from a lyophilized solid form with a diluent. The dose of the mixture is measured by the amount of collagenase present without regard to diluent, and may comprise about 0.1 mg to about 20 mg in one or more injections. In another embodiment, the dose administered is about 0.06 mg, 0.48 mg, 0.84 mg, 1.68 mg, 2.52 mg, 3.36 mg, 4.2 mg, 5.04 mg, 5.88 mg, 6.72 mg, 7.56 mg, or 8.4 mg in one or more injections.

For instance, about 0.06 mg, 0.48 mg, 0.84 mg, or 1.68 mg is administered in about 12 divided injections. The volume of collagenase composition injected may range from 0.01 mL to 3 mL per injection, or total about 0.2 mL to 150 mL per treatment visit. In a specific embodiment, the above doses are to a collagenase composition comprising CCH. In another embodiment, the above doses are to a collagenase composition having one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another embodiment, about 0.84 mg of CCH is injected in about 12 equally divided injections per treatment area (about 0.07 mg×12 injections=about 0.84 mg). In some cases, such treatment with 0.84 mg occurs every 10-40 days for 2, 3, 4 or 5 treatment visits. In other cases, more than one treatment area is injected with 0.84 mg in one treatment visit or every 10-40 days for 2, 3, 4 or 5 treatment visits. In other embodiments, there are more than 5 treatment visits.

In another aspect, the amount of collagenase that may be injected to a treatment area(s) is about 0.001 mg to 20 mg collagenase per treatment visit in one or more injections, e.g., the dose is divided equally into about 3 to about 100 injections. The collagenase is in liquid form, or is reconstituted from a lyophilized solid with a diluent. The dose of collagenase is measured by the amount of collagenase without regard to diluent, and may comprise about 0.1 mg to 1 mg, or 0.25 mg to 0.75 mg, or 0.1 mg to 2 mg, or 0.25 mg to 1.75 mg, or 0.5 mg to 1 mg, 0.1 mg to 3 mg, or 0.25 mg to 2.75 mg, or 0.5 mg to 2.5 mg, or 0.75 mg to 2.25 mg, or 1 mg to 2 mg, or 0.1 mg to 4 mg, or 0.25 mg to 3.75 mg, or 0.5 mg to 3.5 mg, or 0.75 mg to 3 mg, or 1 mg to 3 mg. In other embodiments, the dose is about 0.001 mg, 0.01 mg, 0.04 mg, 0.05 mg, 0.07 mg, 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.25 mg, 2.5 mg, 2.75 mg, 3 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 11 mg, 12, mg, 13 mg, 14 mg, 15 mg, 16 mg, 17 mg, 18 mg, 19 mg, or 20 mg in one or more injections.

In another embodiment, the dose administered is about 0.06 mg, 0.48 mg, 0.84 mg, 1.68 mg, 2.52 mg, 3.36 mg, 4.2 mg, or 5.04 mg in one or more injections. In another example, about 0.06 mg, 0.48 mg, 0.84 mg, 1.68 mg, 2.52 mg, 3.36 mg, 4.2 mg, or 5.04 mg is administered in about 12 divided injections to a treatment area. In other examples, the dose of collagenase is divided into 3 or more injections. The volume of collagenase composition injected may range from 0.01 mL to 3 mL per injection, or total about 1 mL to 150 mL per treatment visit.

In one aspect, the AUX I and II mixture described above (“CCH”) may be injected in an amount of about 0.01 mg to 10 mg collagenase per treatment visit in one or more injections, e.g., the dose is divided equally into about 3 to about 50 injections. The collagenase may be a liquid or may be reconstituted from a lyophilized form with a diluent. The dose of the mixture is measured by the amount of collagenase without regard to diluent, and may comprise about 0.1 mg to 1 mg, or 0.25 mg to 0.75 mg, or 0.1 mg to 2 mg, or 0.25 mg to 1.75 mg, or 0.5 mg to 1 mg, 0.1 mg to 3 mg, or 0.25 mg to 2.75 mg, or 0.5 mg to 2.5 mg, or 0.75 mg to 2.25 mg, or 1 mg to 2 mg, or 0.1 mg to 4 mg, or 0.25 mg to 3.75 mg, or 0.5 mg to 3.5 mg, or 0.75 mg to 3 mg, or 1 mg to 3 mg, or about 0.05 mg, 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.25 mg, 2.5 mg, 2.75 mg, 3 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg or 10 mg in one or more injections. In another embodiment, the dose of CCH administered is about 0.06 mg, 0.48 mg, 0.84 mg, or 1.68 mg 2.52 mg, 3.36 mg, 4.2 mg, or 5.04 mg in one or more injections. For instance, about 0.06 mg, 0.48 mg, 0.84 mg, or 1.68 mg 2.52 mg, 3.36 mg, 4.2 mg, or 5.04 mg is administered in 12 injections. The volume of collagenase composition injected may range from 0.01 mL to 3 mL per injection, or total about 1 mL to 80 mL per treatment visit.

The doses of collagenase can also be expressed in mg per injection (again without regard to diluent) such as from about 0.001 mg to 0.5 mg per injection, about 0.01 mg to about 5 mg per injection, or about 0.005 mg to about 0.1 mg, or about 0.005 mg, 0.04 mg, or 0.07 mg per injection.

In certain aspects, the present disclosure contemplates injecting about 500 ABC units to about 000 ABC units per treatment visit, or about 10,000 ABC units to about 25,000 ABC units per treatment visit. In another embodiment, the dose of collagenase per injection is about 50ABC units to about 2,500ABC units, or about 85 ABC units to about 2,000 ABC units, or about 150ABC units to about 1,750ABC units, or about 200ABC units to about 1,500ABC units, or about 300 ABC units to about 1,250 ABC units, or about 500 ABC units to about 1,000 ABC units.

In certain embodiments, the doses based on various specific activities are as follows:

| | Doses | | | | | | | | | | | |
|---|---|---|---|---|---|---|---|---|---|---|---|---|
| Specific | 500 SRC | | 700 SRC | | 1000 SRC | | 1500 SRC | | 10,000 ABC | | 15,000 SRC | |
| Activities | Units | | Units | | Units | | Units | | Units | | Units | |
| 500 SRC | 1.00 | mg* | 1.40 | mg | 2.00 | mg | 3.00 | mg | 3.17 | mg | 30.00 | mg |
| Units/mg | | | | | | | | | | | | |
| 700 SRC | 0.71 | mg | 1.00 | mg | 1.43 | mg | 2.14 | mg | 2.27 | mg | 21.43 | mg |
| Units/mg | | | | | | | | | | | | |
| 1000 SRC | 0.50 | mg | 0.70 | mg | 1.00 | mg | 1.50 | mg | 1.59 | mg | 15.00 | mg |
| Units/mg | | | | | | | | | | | | |
| 1500 SRC | 0.33 | mg | 0.47 | mg | 0.67 | mg | 1.00 | mg | 1.06 | mg | 10.00 | mg |
| Units/mg | | | | | | | | | | | | |
| 10,000 ABC | 0.18 | mg† | 0.25 | mg | 0.36 | mg | 0.55 | mg | 0.58 | mg | 5.48 | mg |
| Units/0.58 mg | | | | | | | | | | | | |
| 15,000 SRC | 0.033 | mg | 0.047 | mg | 0.067 | mg | 0.100 | mg | 0.106 | mg | 1.00 | mg |
| Units/mg | | | | | | | | | | | | |

*Milligram calculation from SRC units and specific activity in SRC units/mg is achieved by multiplying the SRC units by the inverse of the specific activity in SRC units/mg. For example, when the dose is 500 SRC units and the specific activity is 500 SRC units/mg, the amount in milligrams equivalent to the 500 SRC units dose is (500 SRC units) * (1/(500 SRC units/mg)) = 1.00 mg.
†Milligram calculation from SRC units and specific activity in ABC units/mg is achieved by multiplying the SRC units by 6.3 ABC units/SRC unit, and then multiplying by the inverse of the specific activity in ABC units/mg. For example, when the dose is 500 SRC units and the specific activity is 10,000 ABC units/0.58 mg, the amount in milligrams equivalent to the 500 SRC units is (500 SRC units) * (6.3 ABC units/SRC unit) * (1/(10,000 ABC units/0.58 mg)) = 0.18 mg.

In certain aspects, the present disclosure contemplates injecting collagenase in an amount of about 5,000 BTC units to about 25,000 BTC units, or about 10,000 BTC units to about 25,000 BTC units, or about 15,000 BTC units, or about 17,500 BTC units, or about 20,000 BTC units, or about 22,500 BTC units, or about 9,175 BTC units, or about 15,817 BTC units.

5. Formulations

The CCH or other collagenase may be in the form of a pharmaceutical formulation comprising the CCH or collagenase and pharmaceutically acceptable excipients. Such excipients may include sterile water or sodium chloride/calcium chloride for injection, pH adjusting agents and stabilizers.

One non-limiting example is XIAFLEX®, supplied commercially by Applicant as single-use glass vials containing 0.9 mg of CCH as a sterile, lyophilized powder for reconstitution. Sterile diluent for reconstitution is also provided in a single-use glass vial. Inactive ingredients include hydrochloric acid, sucrose, and tromethamine. The diluent contains calcium chloride dihydrate in 0.9% sodium chloride. XIAFLEX® Prescribing Information (2018).

In another embodiment, CCH is a sterile lyophilized powder comprising the 0.92 mg CCH, sucrose, Tris, mannitol, and hydrochloric acid, in a 5-mL vial. A sterile diluent for reconstitution may comprise water for injection, normal saline, or 0.6% sodium chloride and 0.03% calcium chloride dehydrate in water for injection filled into individual 5 mL vials.

The collagenase or CCH may be filled into other size vials, e.g., 10 mL, 15 mL, 20 mL, or 30 mL. Other pH adjusting agents, sugars, polyols and stabilizing agents may be found in Rowe et al., Handbook of Pharmaceutical Excipients (5^{th} Ed.).

6. Methods of Treatment: Injection Techniques and Dosing Regimens

The foregoing collagenase compositions are useful in methods to treat or reduce the severity of cellulite in human subjects. The present disclosure relates to a method to reduce the severity of cellulite in a human patient, comprising: providing a composition comprising at least one collagenase; and injecting a therapeutically effective amount of the composition to one or more dimples, wherein the patient demonstrates a reduction in the severity of cellulite compared to a pretreatment baseline level of severity. As further detailed below, the composition may be administered by various injection techniques and the efficacy measured by a number of scales and other measurement tools.

Applicant has previously described related methods of treatment and hereby incorporates by reference in their entireties its patent applications PCT Patent Application PCT/US2018/020551 (published as WO2018/160905 on Sep. 7, 2018); U.S. Provisional Appl. No. 62/697,376 entitled “Injection Techniques for the Treatment of Edematous Fiberosclerotic Panniculopathy,” filed on Jul. 12, 2018; U.S. Provisional Appl. No. 62/733,046 entitled “Composition and Method of Administering a Single Dose of Collagenase in Four Quadrants to Treat Edematous Fiberosclerotic Panniculopathy,” filed on Sep. 18, 2018; and U.S. Patent Appl. Pub. No. US20180327731 entitled “method of Producing Collagenase, filed on Mar. 28, 2018.

The administration of the collagenase compositions described herein may be bilaterally (two thighs or two buttocks) or to all 4 quadrants (both buttocks and both thighs) in a single subject during a treatment visit. Such treatment visits may occur every 10-40 days for 2, 3, 4 or 5 treatment visits over a one-year period.

A general overview of the various injection parameters for collagenase and related techniques for treatment of patients is provided in Table 15.

TABLE 15
An Overview of the Various Injection Parameters for Collagenase
and Related Techniques for Treatment of Patients

| Parameters | Approximate Ranges and Descriptions |
|---|---|
| Total dose per treatment visit (mg) | 1 mg to 20 mg collagenase |
| Total dose per treatment visit (fSRC units) | Collagenase or CCH: 5,000 to 600,000 |
| Total dose per treatment visit (fGPA units) | Collagenase or CCH: 175,000 to 10 million |
| Total dose per treatment visit (ABC units) | Collagenase or CCH: 5,000 to 500,000 |
| Total dose per treatment visit (SRC units) | Collagenase or CCH: 500 to 600,000 |
| Total dose per treatment visit (GPA units) | Collagenase or CCH: 100,000 to 8 million |
| Total dose per treatment area (mg) | Collagenase or CCH: 0.07 mg to 5 mg |
| Total dose per treatment area (fSRC units) | Collagenase or CCH: 1,250 to 150,000 |
| Total dose per treatment area (fGPA units) | Collagenase or CCH: 43,750 to 2.5 million |
| Total dose per treatment area (ABC units) | Collagenase or CCH: 1,250 to 125,000 |
| Total dose per treatment area (SRC units) | Collagenase or CCH: 1,250 to 150,000 |
| Total dose per treatment area (GPA units) | Collagenase or CCH: 25,000 to 2 million |
| Dose per injection | 0.0001 mg to 1 mg, or 0.0002 mg to 0.5 mg, |
| | or 0.00029 mg to 0.01 mg, or 0.01 mg to 2 |
| | mg, or 0.025 mg to 1.5 mg, or 0.05 mg to 1 |
| | mg, or 0.07 mg to 0.25 mg |
| Total injection volume per treatment visit | 0.1 mL to 150 mL, or 0.2 mL to 7.2 mL; or 5 |
| (mL) | mL to 50 mL, or 20 mL to 40 mL, or 28 mL |
| | to 36 mL |
| Total injection volume per treatment area | 0.1 mL to 36 mL |
| (mL) | |
| Total injection volume per dimple (mL) | 0.01 mL to 10 mL, or 1.2 mL, or 1.5 mL, or |
| | 0.3 mL. |
| Number of injection aliquots per injection | 1 to 12 |
| Number of injections per dimple | 1 to 5 |
| Number of aliquots per dimple | 1 to 60 |
| Volume of each aliquot | 0.01 mL to 36 mL |
| Collagenase concentration per injection | 0.01 mg/mL to 1 mg/mL, or 0.04 mg/mL to |
| | 0.9 mg/mL, or 0.06 mg/mL to 0.75 mg/mL, or |
| | 0.23 mg/mL, or 0.001 mg/mL to 10 mg/mL |
| Angle of subcutaneous injection into the | 5 to 175 degrees, or 30 to 150 degrees, or 45 |
| dimple | to 135 degrees, or 75 to 105 degrees, or 90 |
| | degrees |
| Depth of injection from skin surface | ⅛ inch to 2 inches |
| Needle gauge and length | 25 to 32 gauge; ⅛ inch to 3 inches needle |
| Number of injections per treatment visit | 3 to 175 injections |
| Length of time between treatment sessions | 7 to 100 days |
| (days) | |
| Dilution factor | 1x, 2x, 3x, 4x, 5x, 6x, 7x, 8x, 9x or 10x |
| Amount lyophilized collagenase in vial | 1 mg to 50 mg |
| Diluent | 0.9% NaCl/0.03% CaCl₂, or 0.6% NaCl/ |
| | 0.03% CaCl₂ |
| Osmolality of reconstituted product | 50 to 1,000 mOsm/kg |
| Amount of collagenase in vial (liquid | 1 mg to 50 mg |
| product) | |
| Vial size | 2 mL to 50 mL, or 5 mL, or 7.5 mL, or 10 |
| | mL, or 15 mL, or 20 mL, or 30 m, or 40 mL, |
| | or 50 mL |
| pH of collagenase | 4 to 9 |

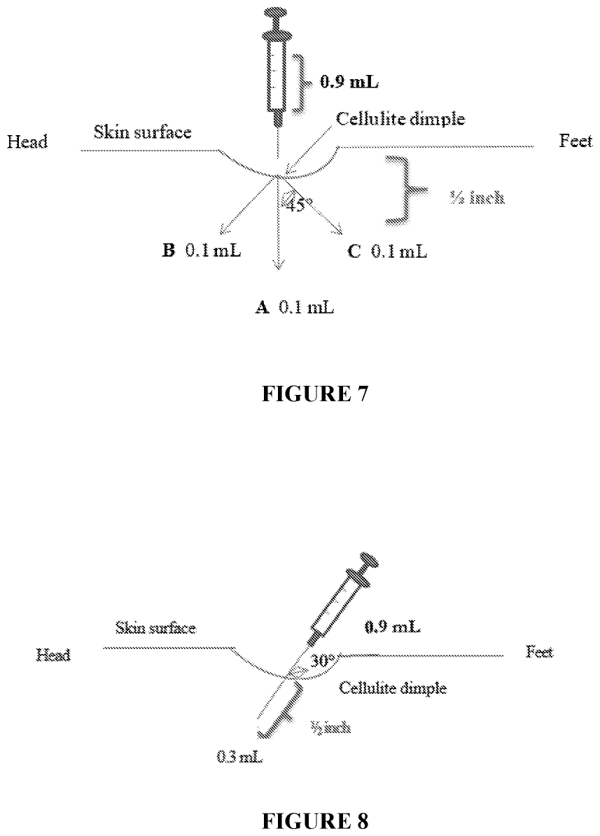

Five non-limiting examples of the injection techniques and treatments contemplated by the present disclosure are outlined in Table 16, which correspond to the injection techniques illustrated in FIGS. 7-11.

TABLE 16
Volume and Concentration of Collagenase Injections by Treatment Arm

| | | Dose/ | | Drug | | Total volume/treatment |
|---|---|---|---|---|---|---|
| Treatment | Area | injection | Volume/injection | concentration/injection | | visit |
| Treatment I: Shallow | (buttocks) | 0.07 mg | 0.3 mL | 0.23 | mg/mL | 7.2 mL |
| Injection, 3 aliquots | (thighs) | | (given as 3- 0.1 mL | | | (12 injections/treatment |
| | | | aliquots) | | | area x 2 treatment areas x |
| | | | | | | 0.3 mL) |
| Treatment II: Shallow | (buttocks) | 0.07 mg | 0.3 mL | 0.23 | mg/mL | 7.2 mL |
| injection, 1 aliquot | (thighs) | | (given as 1- 0.3 mL | | | (12 injections/treatment |
| | | | aliquot) | | | area x 2 treatment areas x |
| | | | | | | 0.3 mL) |
| Treatment III: Deep | (buttocks) | 0.07 mg | 0.3 mL | 0.23 | mg/mL | 7.2 mL |
| injection, 1 aliquot | (thighs) | | (given as 1- 0.3 mL | | | (12 injections/treatment |
| | | | aliquot) | | | area x 2 treatment areas x |
| | | | | | | 0.3 mL) |
| Treatment IV: Deep and | (buttocks) | 0.07 mg | 1.5 mL | 0.047 | mg/mL | 36 mL |
| shallow injections, | (thighs) | | (given as 5- 0.3 mL | | | (12 injections/treatment |
| 5 aliquots | | | aliquots) | | | area x 2 treatment areas x |
| | | | | | | 1.5 mL) |
| Treatment V: Shallow | (buttocks) | 0.07 mg | 1.2 mL | 0.060 | mg/mL | 28.8 mL |
| injection, 4 aliquots | (thighs) | | (given as 4- 0.3 mL | | | (12 injections/treatment |
| | | | aliquots) | | | area x 2 treatment areas x |
| | | | | | | 1.2 mL) |

Further details regarding Treatments I to V are set forth below.

Treatment I: Collagenase Shallow Injection, 3 Aliquots

As illustrated in FIG. 7, in this example (“Treatment I”), collagenase is injected subcutaneously and perpendicular to the long axis of a dimple while the subject lies in a prone position. Each injection comprises a single skin injection of collagenase as three 0.1 mL aliquots (for a total injection volume of 0.3 mL). During each treatment visit, 8 syringes (4 syringes per treatment area) are prepared for dosing. Each syringe contains 0.9 mL of collagenase composition (3 injections in each syringe). The dose per subject may vary from a total dose of about 0.5 mg to about 5 mg collagenase per treatment area.

More specifically, the following procedure is followed:
With the needle positioned perpendicular to skin surface and perpendicular to the long axis of a dimple if the dimple is an elongated trough-like dimple (Position A), push the needle all the way in (½ inch) and inject 0.1 mL of collagenase composition by gently pushing the syringe plunger. In most cases, the plane containing injection points A, B and C will be parallel to the long axis of the subject's body.
Withdraw the needle slightly and reposition it at an angle of approximately 45 to the skin surface and towards the subject's head (Position B), push the needle all way in and inject 0.1 mL of collagenase composition by gently pushing the syringe plunger. Position B is preferably towards the head of the subject.
Withdraw the needle slightly and reposition it at an angle of approximately 45° to the skin surface and towards the subject's feet (Position C), push the needle all the way in and inject 0.1 mL of collagenase composition by gently pushing the syringe plunger. Position C is preferably towards the feet of the subject.
Withdraw the needle completely from the injection site and proceed to next injection site.
A syringe with 0.9 mL collagenase composition is sufficient for 3 injection sites.
Use four (4) 0.9 mL syringes in each treatment area (each buttock or each thigh) to administer a total of 12 injections of 0.3 mL (3 aliquots of 0.1 mL each) at 12 injection sites.
A total of 24 injections is administered across the 2 treatment areas (2 buttocks or 2 thighs) at each treatment visit.

Treatment II: Collagenase Shallow Injection, 1 Aliquot

As illustrated in FIG. 8, in this example (“Treatment II”), collagenase is injected subcutaneously while the subject is in a prone position. Each injection comprises a single skin injection of collagenase as a single shallow injection of a 0.3 mL aliquot. During each treatment visit, 8 syringes (4 syringes per treatment area) are prepared for dosing. Each syringe contains 0.9 mL of collagenase (3 injections in each syringe). The dose per subject may vary from a total dose of about 0.5 mg to 5 mg of collagenase in each treatment area.

During each treatment visit, 8 syringes (4 syringes per treatment area) are prepared for dosing. Each syringe contains 0.9 mL of collagenase composition (3 injections in each syringe). More specifically, the following procedure is followed:
With the needle positioned at approximately 30 to the skin surface at the injection site and directed towards subject's head, push the needle all the way in (½ inch) and inject 0.3 mL of collagenase composition by gently pushing the syringe plunger.
Withdraw the needle completely from the injection site and proceed to the next injection site.
A syringe with 0.9 mL collagenase composition will be sufficient for 3 injection sites.
Use four (4) 0.9 mL syringes for each treatment area (each buttock or each thigh) to administer a total of 12 injections of 0.3 mL (3 aliquots of 0.1 mL each) at 12 injection sites.
A total of 24 injections is administered across the 2 treatment areas (2 buttocks or 2 thighs) at each treatment visit.

Treatment III: Collagenase Deep Injection, 1 Aliquot

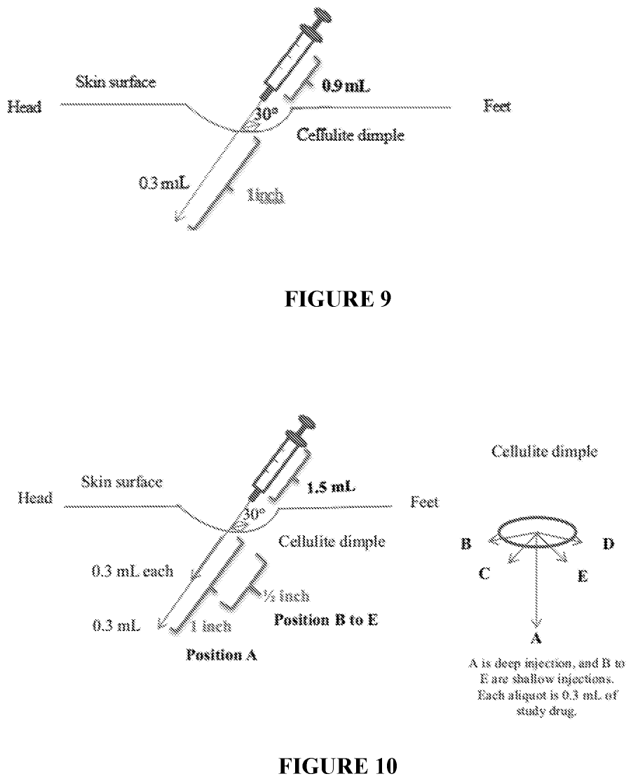

As illustrated in FIG. 9, in this example (“Treatment III”), collagenase is injected subcutaneously while the subject is in a prone position. Each injection comprises a single skin injection of collagenase composition as a single deep injection of a 0.3 mL collagenase composition aliquot. During each treatment visit, 8 syringes (4 syringes per treatment area) are prepared for dosing. Each syringe contains 0.9 mL of collagenase (3 injections in each syringe). The dose per subject may vary from a total dose of about 0.5 mg to about 5 mg collagenase per treatment area.

More specifically, the following procedure is followed:
With the needle positioned at an angle of approximately 300 to the skin surface at the injection site and directed towards subject's head, push the needle all the way in (1 inch) and inject a single aliquot 0.3 mL of collagenase composition by gently pushing the syringe plunger.
With the needle positioned at an angle of approximately 30° to the skin surface at the injection site and directed towards subject's head, push the needle all the way in (1 inch) and inject a single aliquot 0.3 mL of collagenase composition by gently pushing the syringe plunger.
A syringe with 0.9 mL collagenase composition will be sufficient for 3 injection sites.
Use four (4) 0.9 mL syringes in each treatment area (each buttock or each thigh) to administer a total of 12 injections of 0.3 mL (single 0.3 mL aliquots) at 12 injection sites.
Discard the used needles and syringes.
A total of 24 injections is administered across 2 treatment areas (2 buttocks or 2 thighs) at each treatment visit.

Treatment IV: Collagenase Deep and Shallow Injections, 5 Aliquots

As illustrated in FIG. 10, in Treatment IV, collagenase is injected subcutaneously while the subject lies in a prone position. Each injection comprises a single skin injection of collagenase as five 0.3 mL (for a total injection volume of 1.5 mL). During each treatment visit, 24 syringes (12 syringes per treatment area) are prepared for dosing. Each syringe contains 1.5 mL of collagenase (5 aliquots of 0.3 mL, for each injection, in each syringe). The dose per subject may vary from a total dose of about 0.5 mg to about 5 mg per treatment area.

During each treatment visit, 24 syringes (12 syringes per treatment area) are prepared for dosing. Each syringe contains 1.5 mL of collagenase composition (5 aliquots of 0.3 mL, for each injection, in each syringe). More specifically, the following procedure is followed:
With the needle positioned at an angle of approximately 30 to the skin surface at the injection site and directed towards subject's head, push the needle all the way in (1 inch) and inject 0.3 mL of collagenase composition by gently pushing the syringe plunger.
Gently withdraw half the length of the needle (½ inch), maintaining the 30° angle of the needle to skin surface, and reposition it towards one side of the subject (Position B). Inject 0.3 mL collagenase composition by gently pushing the syringe plunger.
Maintain the 30° angle of the needle to the skin surface, reposition the needle to midway between Position A and B (Position C, towards subject's shoulder) and inject 0.3 mL of collagenase composition by gently pushing the syringe plunger.
Maintain the 30° angle of the needle to the skin surface, reposition the needle exactly opposite to Position B (Position D), and inject 0.3 mL of collagenase composition by gently pushing the syringe plunger.
Maintain the 30° angle of the needle to the skin surface, reposition the needle to midway between Position A and D (Position E, towards subject's other shoulder) and inject 0.3 mL of collagenase composition by gently pushing the syringe plunger.
A 3 mL syringe with 1.5 mL of collagenase composition will be sufficient for one injection site.
Use twelve (12) 3 mL syringes in each treatment area (each buttock or each thigh) to administer a total of 12 injections of 1.5 mL (total of 18.0 mL, 5 aliquots 0.3 mL each) at 12 injection sites.
A total of 24 injections is administered across the 2 treatment areas (2 buttocks or 2 thighs) at each treatment visit.

Treatment V: Collagenase Shallow Injections, 4 Aliquots

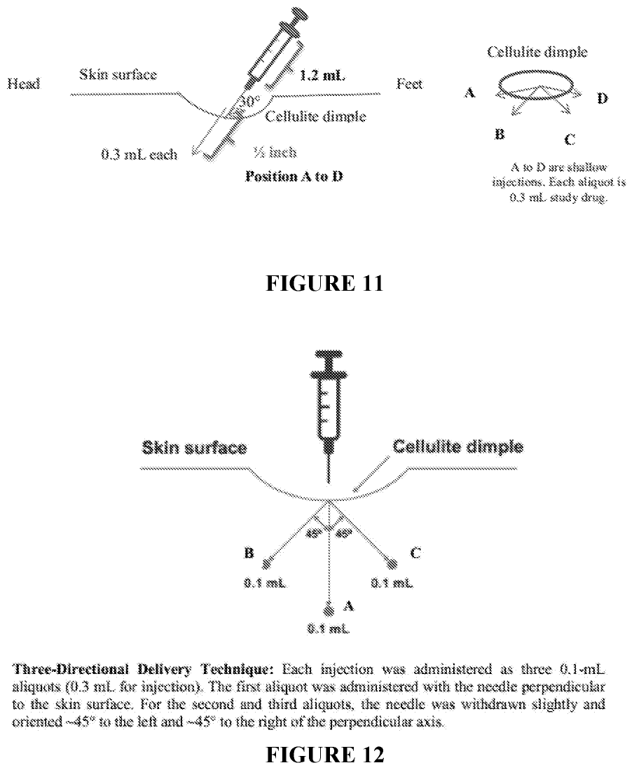

As illustrated in FIG. 11, in this example (“Treatment V”), collagenase is injected subcutaneously while the subject lies in a prone position. Each subject will receive a single skin injection of collagenase as four 0.3 mL aliquots (for a total injection volume of 1.2 mL). During each treatment visit, 24 syringes (12 syringes per treatment area) are prepared for dosing. Each syringe will contain 1.2 mL of collagenase (4 aliquots of 0.3 mL each). The dose per subject may vary from a total dose of about 0.5 mg to about 5 mg per treatment area.

During each treatment visit, 24 syringes (12 syringes per treatment area) were prepared for dosing. Each syringe contained 1.2 mL of collagenase composition (4 aliquots of 0.3 mL each). More specifically, the following procedure is followed:
With the needle positioned at an angle of approximately 300 to skin surface at the injection site and directed towards one side of the subject, push the needle all the way in (½ inch), and inject 0.3 mL of collagenase composition by gently pushing the syringe plunger.
Maintain the 30° angle of the needle to the skin surface, reposition the needle to approximately 60° from Position A and towards the subject's shoulder (Position B), and inject 0.3 mL of collagenase composition by gently pushing the syringe plunger.
Maintain the 30° angle of the needle to the skin surface, reposition the needle to approximately 60° from Position B and towards subjects other shoulder (Position C) and inject 0.3 mL of collagenase composition by gently pushing the syringe plunger.
Maintain the 30° angle of the needle to the skin surface, reposition the needle opposite to position A and towards other side of the subject (Position D) and inject 0.3 mL of collagenase composition by gently pushing the syringe plunger.
A 3 mL syringe with 1.2 mL of collagenase composition will be sufficient for one injection site.
Use twelve (12) 3 mL syringes (with 1.2 mL collagenase composition in each) in each treatment area (each buttock or each thigh) to administer a total of 12 injections of 1.2 mL each (total of 14.4 mL, 4 aliquots of 0.3 mL each) at 12 injection sites.
A total of 24 injections is administered across 2 treatment areas.

In a further embodiment, the collagenase is injected into an affected area as illustrated in FIG. 12. The spacing of the injections can vary from between about 0.1 cm to about 15 cm, or about 1 cm to about 10 cm, or about 0.5 cm to about 2 cm. As shown in FIG. 12, each injection is administered as three 0.1 mL aliquots (0.3 mL per injection). The first aliquot is administered with the needle perpendicular to the skin surface. For the second and third aliquots, the needle is withdrawn slightly and oriented about 45 to the left and about 45° to the right of the perpendicular axis.

Treatments I to V shown in FIGS. 7-11 and the injection technique shown in FIG. 12 may use the different doses, angle of injections, volumes, number of syringes, depth of injection and other parameters detailed in Tables 15 and 16. All such variations are encompassed by the present disclosure.

In another example, about 0.84 mg of CCH is injected in about 12 equally divided injections to an affected area such as a quadrant (i.e., the right or left buttock or right or left thigh) (about 0.07 mg×12 injections=about 0.84 mg CCH). In some cases, such treatment with 0.84 mg occurs every 10-40 days for 2, 3, 4 or 5 treatments. In other cases, more than one affected area or quadrant is injected with 0.84 mg every 10-40 days for 2, 3, 4 or 5 treatments.

In certain embodiments, patients are administered collagenase as shown in Table 17.

TABLE 17
Collagenase Dose and Volume

| | | | | Injection | |
|---|---|---|---|---|---|
| Dose per | Injection | | | Volume (mL) | |
| Each Injection^{a}/ | Volume per | Number of | | per Each | |
| Number of | Each | Injections at Each | Dose (mg) at Each | Treatment | Cumulative EFP |
| Subjects | Injection | Treatment Visit | Treatment Visit | Visit | Dose |
| 0.07 mg | 0.3 mL | 12 per treatment | 0.84 mg per | 3.6 mL per | 5.04 mg |
| | | area × 2 treatment | treatment area × 2 | treatment area × | (3 treatment visits × |
| | | areas = 24 | treatment areas = | 2 treatment | 0.84 mg per |
| | | | 1.68 mg | area = 7.2 mL | treatment area × 2 |
| | | | (12 injections per | (24 injections × | treatment areas) |
| | | | treatment area × | 0.3 mL) | |
| | | | 0.07 mg/injection × | | |
| | | | 2 treatment areas) | | |

^{a}Each injection of collagenase is 0.3 mL administered as three 0.1 mL aliquots.

Further, in certain specific embodiments, the parameters for treatment patients are provided in Tables 18 and 19.

TABLE 18
CCH Lyophilized Formulation Parameters

| | Cellulite - | | Cellulite- | |
|---|---|---|---|---|
| | Buttock/treatment | | thigh/treatment | |
| | area | | area | |
| Amount cake in vial | 0.92 | mg CCH | 0.92 | mg CCH |
| Syringe | 30 gauge × ½ inch | | 30 gauge × 1 inch | |
| Diluent | 0.6% NaCl/0.03% CaCl₂ | | 0.6% NaCl/0.03% CaCl₂ | |
| Osmolality of | ~235-315 | mOsm/kg | ~227 | mOsm |
| Reconstituted | | | | |
| Product | | | | |
| Dose applied per | 0.84 mg per treatment | | 0.84 mg per treatment | |
| treatment visit | area | | area | |
| Concentration to | 0.233 | mg/mL | 0.05 | mg/mL |
| be injected | | | | |

| | | |
|---|---|---|
| Vial size | 5 mL or 10 mL | 5 mL or 10 mL |

TABLE 19
CCH Dilution Parameters

| | | Target | | |
|---|---|---|---|---|
| | | Concentration | | Osmolality |
| | Sample | Mg/mL | pH | (mOsM/kg) |
| Diluent A | CCH 1X | 0.23 | 8.1 | 362 to 402 |
| (0.9% NaCl, | reconstitution | | | |
| 0.03% | CCH 2X | 0.12 | 7.8 | 329 to 358 |
| CaCl₂) | dilution | | | |
| | CCH 4X | 0.06 | 7.4 | 310 to 336 |
| | dilution | | | |
| | CCH 8X | 0.03 | 7.2 | 301 to 325 |
| | dilution | | | |
| Diluent B | CCH 1X | 0.23 | 8.0 | 267 to 300 |
| (0.6% NaCl, | reconstitution | | | |
| 0.03% | CCH 2X | 0.12 | 7.8 | 235 to 256 |
| CaCl₂) | dilution | | | |
| | CCH 4X | 0.06 | 7.5 | 216 to 234 |
| | dilution | | | |
| | CCH 8X | 0.03 | 7.3 | 208 to 223 |
| | dilution | | | |

In one embodiment, the osmolality of reconstitution product is about 50 to about 1,000, about 100 to about 900, about 200 to about 800, about 300 to about 700, about 400 to about 600, about 50, about 100, about 150, about 200, about 250, about 300, about 350, about 400, about 450, about 500, about 550, about 600, about 650, about 700, about 750, about 800, about 850, about 900, about 950, or about 1,000 mOsm/kg. In yet another embodiment, the osmolality of reconstitution product is about 512 mOsm/kg, about 275 mOsm/kg, about 281mOsm/kg, or about 227mOsm/kg.

In addition to the methods above, the current disclosure provides a method of treating or reducing EFP in a subject in need thereof, wherein the method provides at least one of the following advantages over common procedures and treatments of EFP:
a. ease of use by a physician;
b. shorter time to treat;
c. unexpected efficacy in light of physicians' general view that improvements for aesthetic conditions is difficult to obtain;
d. no need to use hyaluronidase;
e. no need to use heat;
f. no need to use lasers;
g. no subscision;
h. no need for anesthetic (despite bruising);
i. no need for compressive garments; and
j. no vacuum is used.

In another embodiment, the method of treatment or reducing cellulite places no cap on the severity of the cellulite to be treated, e.g., the treatment with collagenase is safe and effective regardless of the prevalence or severity of cellulite.

### F. Phase 4-Therapeutic Endpoints and Measurements of Efficacy

The treatments described herein are effective in treating cellulite by a number of measures described below. As used herein, “Day” means the study day; thus, for example, Day 22 is 21 days after the first injection, etc. Further, in one example “Day” is +/−7 days.

### 1. Efficacy as Measured by CR-PCSS and PR-PCSS

An improvement for the individual patient at any visit is an improvement of at least 1 level or 1 rating from baseline or any previous score or rating. An improvement for a group of patients at any visit is an improvement of about 0.1 in the mean score or rating from the baseline or any previous mean score or rating. A responder is any patient showing at least a 25% improvement of maximum total score or rating from baseline. In certain embodiments, the treatment methods detailed above result in one or more of the following efficacy endpoints as measured by CR-PCSS and/or PR-PCSS:

1. An improvement of at least 0.1 in CR-PCSS and/or PR-PCSS rating over baseline.
2. An improvement in severity at Day 22, 43, 71, 90, 180, 365 or 730 from baseline (pretreatment “Day 1”) of at least 2 levels of severity in the CR-PCSS as assessed live by the clinician of the target thigh.
3. An improvement in severity at Day 22, 43, 71, 90, 180, 365 or 730 from baseline (Day 1) of at least 2 levels of severity in the PR-PCSS as assessed by the subject while viewing the digital image of the target thigh.
4. An improvement demonstrated by a 2-level composite response at Day 22, 43, 71, 90, 180, 365 or 730 defined as a subject with an improvement from baseline of at least 2 levels of severity in the CR-PCSS and an improvement from baseline of at least 2 levels of severity in the PR-PCSS.
5. An improvement in severity at Day 22, 43, 71, 90, 180, 365 or 730 from baseline of at least 1 level of severity in the CR-PCSS as assessed live by the clinician of the target thigh.
6. An improvement in severity at Day 22, 43, 71, 90, 180, 365 or 730 from baseline of at least 1 level of severity in the PR-PCSS as assessed by the subject while viewing the digital image of the target thigh.
7. An improvement demonstrated by a 1-level composite response at Day 22, 43, 71, 90, 180, 365 or 730 defined as a subject with an improvement from baseline of at least 1 level of severity in the CR-PCSS and an improvement from baseline of at least 1 level of severity in the PR-PCSS.
8. In a population of patients who all had CR-PCSS ratings of moderate or severe, the improvement in at least one treatment area was statistically significant compared to placebo wherein the improvement is one or more of Nos. 1 to 7 above.
9. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
10. At Day 180, the improvement seen in the CR-PCSS rating from baseline was consistent on the right and left thighs.
11. In a population of patients who all have CR-PCSS ratings of moderate or severe, the median time to the earliest 2-level CR-PCSS and/or PR-PCSS improvement in at least one treatment area is about 50 days, or 60 days, or 70 days, or 80 days, or 90 days.
12. In a population of patients who all have CR-PCSS ratings of moderate or severe, the median time to the earliest 1-level CR-PCSS and/or PR-PCSS improvement in at least one treatment area is about 15 days, or 20 days, or 30 days, or 40 days, or 50 days, or 60 days, or 70 days, or 80 days, or 90 days.
13. In a population of patients who all have CR-PCSS ratings of moderate or severe, the mean subject CR-PCSS and/or PR-PCSS scores separate from placebo 21 days after the first treatment and demonstrate continuous and significant improvement after subsequent treatments.
14. In a population of patients who all have CR-PCSS ratings of moderate or severe, the percentage of the subjects who have a 2-level composite response as measured by CR-PCSS and/or PR-PCSS in at least one treatment area at Day 71 is from about 1% to 10%, 10% to 20%, 20% to 30%, 30% to 40%, 40% to 50%, or greater than 50%.
15. In a population of patients who all have CR-PCSS ratings of moderate or severe, the percentage of the subjects who have a 1-level composite response as measured by CR-PCSS and/or PR-PCSS in at least one treatment area at Day 71 is from about 1% to 10%, 10% to 20%, 20% to 30%, 30% to 40%, 40% to 50%, or greater than 50%.
16. In a population of patients who all have CR-PCSS ratings of moderate or severe, over one-third, or one-half, or two-thirds, or three-fourths of the patients have at least a 1-level CR-PCSS and/or at least a 1-level PR-PCSS responses in at least one treatment area by Day 71 post-treatment wherein the CR-PCSS results are independent of age, BMI or skin color.
17. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 17 above.

In another instance, the injection of about 1 mg to about 20 mg of collagenase according to one of Treatments I to V results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 17 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 17 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In other embodiments, in response to the above treatments, a patient is a 2 level CR-PCSS responder who shows improvement in CR-PCSS rating of at least 2 levels from baseline (change of −2, −3, or −4) at an evaluation time point. A 1 level CR-PCSS responder is a patient exhibiting improvement in CR-PCSS rating of at least 1 level from baseline (change of −1, −2, −3, or −4) at an evaluation time point. A patient is a 2 level PR-PCSS responder who shows improvement in PR-PCSS rating of at least 2 levels from baseline (change of −2, −3, or −4) at an evaluation time point. A 1 level PR-PCSS responder is a patient exhibiting improvement in PR-PCSS rating of at least 1 level from baseline (change of −1, −2, −3, or −4) at an evaluation time point. In other aspects, a 2 level composite responder is a patient who is both a 2-level PR-PCSS responder and a 2-level CR-PCSS responder at an evaluation time point. A 1 level composite responder is a patient who is both a 1-level PR-PCSS responder and a 1-level CR-PCSS responder at an evaluation time point.

2. Efficacy as Measured by Hexsel Cellulite Severity Scale (Hexsel CSS)

In Hexsel CSS, an improvement for the individual patient at any visit is an improvement of at least 1 level or 1 rating from baseline or any previous score. An improvement for a group of patients at any visit is an improvement of about 0.1 in the mean Hexsel CSS score or rating from the baseline or any previous mean Hexsel CSS score or rating. A responder is any patient showing at least a 25% improvement of maximum total score or rating from baseline. In certain embodiments, the treatment methods detailed above result in one or more of the following efficacy endpoints as measured by Hexsel CSS:

1. In a population of patients who all had baseline Day 1 Hexsel CSS ratings, the injection of collagenase to at least one treatment area during at least one treatment visit resulted in a statistically significant number of such patients meeting one or more of the following efficacy endpoints:
Shift from severe to moderate (from score of 11-15 to 6-10)
Shift from severe to mild (from score of 11-15 to 1-5)
Shift from severe to zero (from score of 11-15 to 0)
Shift from moderate to mild (from score of 6-10 to 1-5)
Shift from moderate to zero (from score of 6-10 to 0)
Shift from mild to zero (from score of 1-5 to 0)
   2. A change in severity at Day 22, 43, 71, 90, 180, 365 or 730 from baseline (pretreatment “Day 1”) of at least 2 levels of severity in the Hexsel CSS as assessed live by the clinician of the target thigh.
   3. A change in severity at Day 22, 43, 71, 90, 180, 365 or 730 from baseline (Day 1) of at least 2 levels of severity in the Hexsel CSS as assessed by the subject while viewing the digital image of the target thigh.
   4. A change demonstrated by a 2-level composite response at Day 22, 43, 71, 90, 180, 365 or 730 defined as a subject with an improvement from baseline of at least 2 levels of severity in the Hexsel CSS by clinician assessment and an improvement from baseline of at least 2 levels of severity in the Hexsel CSS by patient assessment.
   5. A change in severity at Day 22, 43, 71, 90, 180, 365 or 730 from baseline (Day 1) of at least 1 level of severity in the Hexsel CSS as assessed live by the clinician of the target thigh.
   6. A change in severity at Day 22, 43, 71, 90, 180, 365 or 730 from baseline (Day 1) of at least 1 level of severity in the Hexsel CSS as assessed by the subject while viewing the digital image of the target thigh.
   7. A change demonstrated by a 1-level composite response at Day 22, 43, 71, 90, 180, 365 or 730 defined as a subject with an improvement from baseline of at least 1 level of severity in the Hexsel CSS by clinician assessment and an improvement from baseline of at least 1 level of severity in the Hexsel CSS by patient assessment.
   8. In a population of patients who all had Hexsel CSS ratings of moderate or severe, the improvement in at least one treatment area was statistically significant compared to placebo wherein the change is one or more of Nos. 2 to 7 above.
   9. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   10. At day 180, the improvement seen in the Hexsel CSS rating from baseline was consistent on the right and left thighs.
   11. In a population of patients who all have Hexsel CSS ratings of moderate or severe, the median time to the earliest 2-level Hexsel CSS improvement in at least one treatment area is about 50 days, or 60 days, or 70 days, or 80 days, or 90 days.
   12. In a population of patients who all have Hexsel CSS ratings of moderate or severe, the median time to the earliest 1-level Hexsel CSS improvement in at least one treatment area is about 15 days, or 20 days, or 30 days, or 40 days, or 50 days, or 60 days, or 70 days, or 80 days, or 90 days.
   13. In a population of patients who all have Hexsel CSS ratings of moderate or severe, the mean subject Hexsel CSS scores separates from placebo 21 days after the first treatment and demonstrate continuous and significant improvement after subsequent treatments.
   14. In a population of patients who all have Hexsel CSS ratings of moderate or severe, the percentage of the subjects who have a 2-level composite response as measured by Hexsel CSS in at least one treatment area at Day 71 is from about 1% to 10%, 10% to 20%, 20% to 30%, 30% to 40%, 40% to 50%, or greater than 50%.
   15. In a population of patients who all have Hexsel CSS ratings of moderate or severe, the percentage of the subjects who have a 1-level composite response as measured by Hexsel CSS in at least one treatment area at Day 71 is from about 1% to 10%, 10% to 20%, 20% to 30%, 30% to 40%, 40% to 50%, or greater than 50%.
   16. In a population of patients who all have Hexsel CSS ratings of moderate or severe, over one-third, or one-half, or two-thirds, or three-fourths of the patients have at least a 1-level Hexsel CSS responses in at least one treatment area by Day 71 post-treatment wherein the Hexsel CSS results are independent of age, BMI or skin color.
   17. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 17 above.

In another instance, the injection of about 1 mg to about 20 mg of collagenase according to one of Treatments I to V results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 17 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934 Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934 Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

3. Efficacy as Measured by Hexsel Depression Depth Score

In Hexsel Depression Depth Score, an improvement for the individual patient at any visit is an improvement of at least 1 level or 1 rating from baseline or any previous score. An improvement for a group of patients at any visit is an improvement of about 0.1 in the mean Hexsel Depression Depth score or rating from the baseline or any previous mean Hexsel Depression Depth score or rating. A responder is any patient showing at least a 25% improvement of maximum total score or rating from baseline. In certain embodiments, the treatment methods detailed above result in one or more of the following efficacy endpoints as measured by Hexsel Depression Depth Score: 1. In a population of patients who all had baseline Day 1 Hexsel Depression Depth Score ratings, the injection of collagenase to at least one treatment area during at least one treatment visit resulted in a statistically significant number of such patients meeting one or more of the following efficacy endpoints:
Shift from deep depressions (3) to medium depth (2)
Shift from deep depressions (3) to superficial depressions (1)
Shift from deep depressions (3) to no depressions (0)
Shift from medium depth (2) to superficial depressions (1)
Shift from medium depth (2) to no depressions (0)
Shift from superficial depressions (1) to no depressions (0) 2. A change in severity at Day 22, 43, 71, 90, 180, 365 or 730 from baseline (pretreatment “Day 1”) of at least 2 levels of severity in the Hexsel Depression Depth Score as assessed live by the clinician of the target thigh.
   3. A change in severity at Day 22, 43, 71, 90, 180, 365 or 730 from baseline (Day 1) of at least 2 levels of severity in the Hexsel Depression Depth Score as assessed by the subject while viewing the digital image of the target thigh.
   4. A change demonstrated by a 2-level composite response at Day 22, 43, 71, 90, 180, 365 or 730 defined as a subject with an improvement from baseline of at least 2 levels of severity in the Hexsel Depression Depth Score by clinician assessment and an improvement from baseline of at least 2 levels of severity in the Hexsel Depression Depth Score by patient assessment.
   5. A change in severity at Day 22, 43, 71, 90, 180, 365 or 730 from baseline (Day 1) of at least 1 level of severity in the Hexsel Depression Depth Score as assessed live by the clinician of the target thigh.
   6. A change in severity at Day 22, 43, 71, 90, 180, 365 or 730 from baseline (Day 1) of at least 1 level of severity in the Hexsel Depression Depth Score as assessed by the subject while viewing the digital image of the target thigh.
   7. A change demonstrated by a 1-level composite response at Day 22, 43, 71, 90, 180, 365 or 730 defined as a subject with an improvement from baseline of at least 1 level of severity in the Hexsel Depression Depth Score by clinician assessment and an improvement from baseline of at least 1 level of severity in the Hexsel Depression Depth Score by patient assessment.
   8. In a population of patients who all had Hexsel CSS ratings of medium or deep depressions, the improvement in at least one treatment area was statistically significant compared to placebo wherein the change is one or more of Nos. 2 to 7 above.
   9. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   10. At day 180, the improvement seen in the Hexsel Depression Depth Score rating from baseline was consistent on the right and left thighs.
   11. In a population of patients who all have Hexsel Depression Depth Score ratings of medium or deep depressions, the median time to the earliest 2-level Hexsel Depression Depth Score improvement in at least one treatment area is about 50 days, or 60 days, or 70 days, or 80 days, or 90 days.
   12. In a population of patients who all have Hexsel Depression Depth Score ratings of medium or deep depressions, the median time to the earliest 1-level Hexsel Depression Depth Score improvement in at least one treatment area is about 15 days, or 20 days, or 30 days, or 40 days, or 50 days, or 60 days, or 70 days, or 80 days, or 90 days.
   13. In a population of patients who all have Hexsel Depression Depth Score ratings of medium or deep depressions, the mean subject Hexsel Depression Depth Scores separates from placebo 21 days after the first treatment and demonstrate continuous and significant improvement after subsequent treatments.
   14. In a population of patients who all have Hexsel Depression Depth Score ratings of medium or deep depressions, the percentage of the subjects who have a 2-level composite response as measured by Hexsel Depression Depth Score in at least one treatment area at Day 71 is from about 1% to 10%, 10% to 20%, 20% to 30%, 30% to 40%, 40% to 50%, or greater than 50%.
   15. In a population of patients who all have Hexsel Depression Depth Score ratings of medium or deep depressions, the percentage of the subjects who have a 1-level composite response as measured by Hexsel Depression Depth Score in at least one treatment area at Day 71 is from about 1% to 10%, 10% to 20%, 20% to 30%, 30% to 40%, 40% to 50%, or greater than 50%.
   16. In a population of patients who all have Hexsel Depression Depth Score ratings of medium or deep depressions, over one-third, or one-half, or two-thirds, or three-fourths of the patients have at least a 1-level Hexsel Depression Depth Score responses in at least one treatment area by day 71 post-treatment wherein the Hexsel Depression Depth Score results are independent of age, BMI or skin color.
   17. The reduction in the severity of cellulite occurs rapidly within about 7, or 14, or 21, or 30, or 35, or 40, or 45, or 50 days after the first treatment visit.
   18. A change in score from baseline to Day 71 of about −0.1 to about −2.0 one or more treatment areas.
   19. In a statistically significant population of patients, the least squares (LS) mean is from about −0.1 to about −1.5 (95% confidence interval (CI)) for one or more treatment areas.

In another embodiment, the injection of about 1 mg to about 20 mg of collagenase to at least one treatment area during at least one treatment visit results in one or more of the results Nos. 1 to 19 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 19 above.

In another instance, the injection of about 1 mg to about 20 mg of collagenase according to one of Treatments I to V results in one or more of the results Nos. 1 to 19 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 19 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 19 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934 Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 19 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

4. Efficacy as Measured by Likert Scale for Aesthetic Appearance

In the Likert Scale, an improvement for the individual patient at any visit is an improvement of at least 1 level or 1 rating from before treatment or any previous score. An improvement for a group of patients at any visit is an improvement of about 0.1 in the mean Likert score or rating from before treatment or any previous mean Likert score or rating. In certain embodiments, the treatment methods detailed above result in one or more of the following efficacy endpoints as measured by Likert Scale Score: 1. In a population of patients with cellulite, the injection of collagenase to at least one treatment area during at least one treatment visit resulted in a statistically significant number of such patients meeting one or more of the following efficacy endpoints:
A Likert Scale Score of “Improved” (1)
A Likert Scale Score of “Much Improved” (2)
A Likert Scale Score of “Very Much Improved” (3) 2. An improvement in the treatment area appearance at Day 22, 43, 71, 90, 180, 365 or 730 from before treatment of at least 2 levels in the Likert Scale Score as assessed live by the clinician of the target thigh.
   3. An improvement in the treatment area appearance at Day 22, 43, 71, 90, 180, 365 or 730 from before treatment of at least 2 levels in the Likert Scale Score as assessed by the subject while viewing the digital image of the target thigh.
   4. An improvement in the treatment area appearance at Day 22, 43, 71, 90, 180, 365 or 730 from before treatment of at least 1 level in the Likert Scale Score as assessed live by the clinician of the target thigh.
   5. An improvement in the treatment area appearance at Day 22, 43, 71, 90, 180, 365 or 730 from before treatment of at least 1 level in the Likert Scale Score as assessed by the subject while viewing the digital image of the target thigh.
   6. In a population of patients with cellulite, the improvement in Likert Scale Scores in at least one treatment area was statistically significant wherein the improvement is one or more of Nos. 2 to 7 above.
   7. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   8. At Day 180, the improvement seen in the Likert Scale Score rating from baseline was consistent on the right and left thighs.
   9. In a population of patients who have cellulite, the median time to the earliest 2-level Likert Scale Score improvement in at least one treatment area is about 50 days, or 60 days, or 70 days, or 80 days, or 90 days.
   10. In a population of patients who have cellulite, the median time to the earliest 1-level Likert Scale Score improvement in at least one treatment area is about 15 days, or 20 days, or 30 days, or 40 days, or 50 days, or 60 days, or 70 days, or 80 days, or 90 days.
   11. In a population of patients who have cellulite, the mean subject Likert Scale Scores separates from placebo 21 days after the first treatment and demonstrate continuous and significant improvement after subsequent treatments.
   12. In a population of patients who have cellulite, the percentage of the subjects who have a 2-level response as measured by Likert Scale Score in at least one treatment area at Day 71 is from about 1% to 10%, 10% to 20%, 20% to 30%, 30% to 40%, 40% to 50%, or greater than 50%.
   13. In a population of patients who have cellulite, the percentage of the subjects who have a 1-level response as measured by Likert Scale Score in at least one treatment area at Day 71 is from about 1% to 10%, 10% to 20%, 20% to 30%, 30% to 40%, 40% to 50%, or greater than 50%.
   14. In a population of patients who have cellulite, over one-third, or one-half, or two-thirds, or three-fourths of the patients have at least a 1-level Likert Scale Score responses in at least one treatment area by Day 71 post-treatment wherein the Likert Scale Score results are independent of age, BMI or skin color.
   15. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 15 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 15 above.

In another instance, the injection of about 1 mg to about 20 mg of collagenase according to one of Treatments I to V results in one or more of the results Nos. 1 to 15 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 15 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 15 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

5. Dimple Analysis

In certain embodiments, the treatment of cellulite with collagenase(s) decreases dimple size parameters as follows:
Depth: By about 100%, 90%, 80%, 70%, 60%, 50%, 40%, 30%, 25%, or 20%, or 15%, or 10%, or 5%, or 2.5%, or 2%, or 1%
Width: By about 100%, 90%, 80%, 70%, 60%, 50%, 40%, 30%, 25%, or 20%, or 15%, or 10%, or 5%, or 2.5%, or 2%, or 1%
Length: By about 100%, 90%, 80%, 70%, 60%, 50%, 40%, 30%, 25%, or 20%, or 15%, or 10%, or 5%, or 2.5%, or 2%, or 1%
Overall Volume: By about 100%, 90%, 80%, 70%, 60%, 50%, 40%, 30%, 25%, or 20%, or 15%, or 10%, or 5%, or 2.5%, or 2%, or 1%
Surface Area: By about 100%, 90%, 80%, 70%, 60%, 50%, 40%, 30%, 25%, or 20%, or 15%, or 10%, or 5%, or 2.5%, or 2%, or 1%

In some embodiments, the treatments resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other embodiments, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.

In another embodiment, the injection of about 1 mg to about 20 mg of collagenase to at least one treatment area during at least one treatment visit results in a reduction in at least one of the dimple size parameters by at least 5%, or at least 10% or at least 20%, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in a reduction in at least one of the dimple size parameters by at least 5%, or at least 10% or at least 20%.

In another instance, the injection of about 1 mg to about 20 mg of collagenase according to one of Treatments I to V results in a reduction in at least one of the dimple size parameters by at least 5%, or at least 10% or at least 20%, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in a reduction in at least one of the dimple size parameters by at least 5%, or at least 10% or at least 20%.

In certain embodiments, about 1 mg to about 20 mg of an approximate 1:1 ratio of collagenase Type I and collagenase Type II is injected to at least one treatment area during at least one treatment visit and results in a reduction in at least one of the dimple size parameters by at least 5%, or at least 10% or at least 20%, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in a reduction in at least one of the dimple size parameters by at least 5%, or at least 10% or at least 20%, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 6. Efficacy as Measured by Subject Global Aesthetic Improvement Scale (S-GAIS) and Investigator Global Aesthetic Improvement Scale (I-GAIS)

The treatment methods detailed above result in improved responses as measured by S-GAIS and I-GAIS. A 2-level S-GAIS responder is a subject with an S-GAIS rating of at least 2 (+2 or +3) at an evaluation time point. A 1-level S-GAIS responder is a subject with an S-GAIS rating of at least 1 (+1, +2, or +3) at an evaluation time point. A 2-level I-GAIS responder is a subject with an I-GAIS rating of at least 2 (+2 or +3) at an evaluation time point. A 1-level I-GAIS responder is a subject with an I-GAIS rating of at least 1 (+1, +2, or +3) at an evaluation time point. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating.

In certain embodiments, the treatment methods detailed above result in one or more of the following efficacy endpoints as measured by S-GAIS and/or I-GAIS:

1. In a population of patients with cellulite, the injection of collagenase to at least one treatment area during at least one treatment visit results in a statistically significant number of such patients meeting one or more of the following efficacy endpoints:
S-GAIS and/or I-GAIS score of “Improved” (+1)
S-GAIS and/or I-GAIS score of “Much Improved” (+2)
S-GAIS and/or I-GAIS score of “Very Much Improved” (+3)
   2. An improvement at Day 22, 43, 71, 90, 180, 365, or 730 of at least 2 levels in the I-GAIS as assessed live by the clinician of the target thigh.
   3. An improvement at Day 22, 43, 71, 90, 180, 365, or 730 of at least 2 levels in the S-GAIS as assessed by the subject while viewing the digital image of the target thigh.
   4. An improvement demonstrated by a 2-level composite response at Day 22, 43, 71, 90, 180, 365, or 730 defined as a subject with an improvement of at least 2 levels in the I-GAIS by clinician assessment and an improvement of at least 2 levels in the S-GAIS by patient assessment.
   5. An improvement at Day 22, 43, 71, 90, 180, 365, or 730 of at least 1 level in the I-GAIS as assessed live by the clinician of the target thigh.
   6. An improvement at Day 22, 43, 71, 90, 180, 365, or 730 of at least 1 level in the S-GAIS as assessed by the subject while viewing the digital image of the target thigh.
   7. An improvement demonstrated by a 1-level composite response at Day 22, 43, 71, 90, 180, 365, or 730 defined as a subject with an improvement of at least 1 level in the I-GAIS by clinician assessment and an improvement of at least 1 level in the S-GAIS by patient assessment.
   8. In a population of patients with cellulite, the improvement in I-GAIS and/or S-GAIS in at least one treatment area was statistically significant compared to placebo wherein the improvement is one or more of Nos. 2 to 7 above.
   9. The treatment resulted in at least 5% of patients maintaining their level of improvement for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   10. At day 180, the improvement seen in the I-GAIS and S-GAIS rating was consistent on the right and left thighs.
   11. In a population of patients who have cellulite, the median time to the earliest 2-level I-GAIS and/or S-GAIS improvement in at least one treatment area is about 50 days, or 60 days, or 70 days, or 80 days, or 90 days.
   12. In a population of patients who have cellulite, the median time to the earliest 1-level I-GAIS and/or S-GAIS improvement in at least one treatment area is about 15 days, or 20 days, or 30 days, or 40 days, or 50 days, or 60 days, or 70 days, or 80 days, or 90 days.
   13. In a population of patients who have cellulite, the mean subject I-GAIS and/or S-GAIS separates from placebo 21 days after the first treatment and demonstrate continuous and significant improvement after subsequent treatments.
   14. In a population of patients who have cellulite, the percentage of the subjects who have a 2-level composite response as measured by I-GAIS and/or S-GAIS in at least one treatment area at Day 71 is from about 1% to 10%, 10% to 20%, 20% to 30%, 30% to 40%, 40% to 50%, or greater than 50%.
   15. In a population of patients who have cellulite, the percentage of the subjects who have a 1-level composite response as measured by I-GAIS and/or S-GAIS in at least one treatment area at Day 71 is from about 1% to 10%, 10% to 20%, 20% to 30%, 30% to 40%, 40% to 50%, or greater than 50%.
   16. In a population of patients who have cellulite, over one-third, or one-half, or two-thirds, or three-fourths of the patients have at least a 1-level I-GAIS and/or S-GAIS responses in at least one treatment area by Day 71 post-treatment wherein the GAIS results are independent of age, BMI or skin color.
   17. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 17 above.

In another instance, the injection of about 1 mg to about 20 mg of collagenase according to one of Treatments I to V results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 17 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 17 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 7. Efficacy as Measured by PR-CIS

The treatment methods detailed above result in improved responses as measured by PR-CIS. The PR-CIS total score is the sum of the six items on the scales. Item #1 on the PR-CIS asking how happy the subject is about their appearance of cellulite is reversed by subtracting the subject's reported assessment from 10. The PR-CIS total score can range from 0 to 60 with higher numbers reflecting a more negative impact from the cellulite. For PR-CIS total score, a responder is a subject with a reduction in the PR-CIS total score of at least 12 from baseline at an evaluation time point. For individual PR-CIS impact scores, response is an improvement from baseline of at least 2 score intervals at each time point. Further, a responder is any patient showing at least a 20% improvement of maximum total score from baseline. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating. Further, an improvement is a change from baseline of at least 1 level out of 60.

In certain embodiments, the treatment methods detailed above result in one or more of the following efficacy endpoints as measured by PR-CIS:

1. In a population of patients with cellulite, the injection of collagenase to at least one treatment area during at least one treatment visit results in a statistically significant number of such patients meeting one or more of the following efficacy endpoints:
The PR-CIS shows improvement across at least one domain selected from the group consisting of happiness, bother, self-consciousness, embarrassment, looking older, and looking overweight/out of shape
A reduction in the PR-CIS total score of at least 12 from baseline at one or more evaluation time points
PR-CIS impact scores showing improvement from baseline of at least 2 score intervals at one or more evaluation time points
An improvement is a change from baseline of at least 1 level out of 60 2. An improvement in severity at Day 22, 43, 71, 90, 180, 365 or 730 from baseline (pretreatment “Day 1”) of at least 12 points in the PR-CIS for the target thigh.
   3. In a population of patients with cellulite, a statistically significant improvement in severity over placebo at Day 22, 43, 71, 90, 180, 365 or 730 from baseline (pretreatment Day 1) of at least 12 points in the PR-CIS for the target thigh.
   4. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   5. At Day 180, the improvement seen in the PR-CIS rating from baseline was consistent on the right and left thighs.
   6. In a population of patients who have cellulite, the median time to a reduction in the PR-CIS total score of at least 12 from baseline at one or more evaluation time points for at least one treatment area is about 15 days, or 20 days, or 30 days, or 40 days, or 50 days, or 60 days, or 70 days, or 80 days, or 90 days.
   7. In a population of patients who have cellulite, the mean subject PR-CIS score separates from placebo 21 days after the first treatment and demonstrate continuous and significant improvement after subsequent treatments.
   8. In a population of patients who have cellulite, over one-third, or over one-half, or over two-thirds, or over three-fourths of the patients have a reduction in the PR-CIS total score of at least 12 from baseline at one or more evaluation time points in at least one treatment area by Day 71 post-treatment wherein the PR-CIS results are independent of age, BMI or skin color.
   9. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 9 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 9 above.

In another instance, the injection of about 1 mg to about 20 mg of collagenase according to one of Treatments I to V results in one or more of the results Nos. 1 to 9 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 9 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 9 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 9 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 8. Efficacy as Measured by PR-CIS Abbreviated

The treatment methods detailed above result in improved responses as measured by PR-CIS Abbreviated. The PR-CIS Abbreviated total score will be the sum of the five items on the scales. Item #1 on the PR-CIS asking how happy the subject is about their appearance of cellulite will be reversed by subtracting the subject's reported assessment from 10. The PR-CIS Abbreviated total score can range from 0 to 50 with higher numbers reflecting a more negative impact from the cellulite. For PR-CIS Abbreviated total score, a responder is a subject with a reduction in the PR-CIS total score of at least 10 from baseline at an evaluation time point. For individual PR-CIS Abbreviated impact scores, response is an improvement from baseline of at least 2 score intervals at each time point. Further, a responder is any patient showing at least a 20% improvement of maximum total score from baseline. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating. Further, an improvement is a change from baseline of at least 1 level out of 50.

In certain embodiments, the treatment methods detailed above result in one or more of the following efficacy endpoints as measured by PR-CIS Abbreviated:

1. In a population of patients with cellulite, the injection of collagenase to at least one treatment area during at least one treatment visit results in a statistically significant number of such patients meeting one or more of the following efficacy endpoints:
The PR-CIS Abbreviated shows improvement across at least one domain selected from the group consisting of happiness, bother, self-consciousness, embarrassment, looking older, and looking overweight/out of shape
A reduction in the PR-CIS Abbreviated total score of at least 10 from baseline at one or more evaluation time points
PR-CIS Abbreviated impact scores showing improvement from baseline of at least 2 score intervals at one or more evaluation time points
Further, an improvement is a change from baseline of at least 1 level out of 50 2. An improvement in severity at Day 22, 43, 71, 90, 180, 365 or 730 from baseline (pretreatment “Day 1”) of at least 12 points in the PR-CIS Abbreviated for the target thigh.
   3. In a population of patients with cellulite, a statistically significant improvement in severity over placebo at Day 22, 43, 71, 90, 180, 365 or 730 from baseline (pretreatment Day 1) of at least 10 points in the PR-CIS Abbreviated for the target thigh.
   4. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   5. At Day 180, the improvement seen in the PR-CIS Abbreviated rating from baseline was consistent on the right and left thighs.
   6. In a population of patients who have cellulite, the median time to a reduction in the PR-CIS Abbreviated total score of at least 10 from baseline at one or more evaluation time points for at least one treatment area is about 15 days, or 20 days, or 30 days, or 40 days, or 50 days, or 60 days, or 70 days, or 80 days, or 90 days.
   7. In a population of patients who have cellulite, the mean subject PR-CIS Abbreviated score separates from placebo 21 days after the first treatment and demonstrate continuous and significant improvement after subsequent treatments.
   8. In a population of patients who have cellulite, over one-third, or over one-half, or over two-thirds, or over three-fourths of the patients have a reduction in the PR-CIS Abbreviated total score of at least 10 from baseline at one or more evaluation time points in at least one treatment area by Day 71 post-treatment wherein the PR-CIS results are independent of age, BMI or skin color.
   9. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 9 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 9 above.

In another instance, the injection of about 1 mg to about 20 mg of collagenase according to one of Treatments I to V results in one or more of the results Nos. 1 to 9 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 9 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 9 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 9 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 9. Efficacy as Measured by Subject Self-Rating Scale (SSRS)

The treatment methods detailed above result in improvements as measured by SSRS. A SSRS responder is a subject who is at least slightly satisfied (slightly satisfied [4], very satisfied [5], or extremely satisfied [6]) with the appearance of the cellulite on her thighs at Day 71. Further, a responder is any patient showing at least a 17% improvement of maximum total score from baseline. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating. In certain embodiments, the treatment methods result in one or more of the following efficacy endpoints as measured by SSRS Rating:

1. In a population of patients with cellulite, the injection of collagenase to at least one treatment area during at least one treatment visit resulted in a statistically significant number of such patients meeting one or more of the following efficacy endpoints:
A SSRS Rating of “Slightly satisfied”
A SSRS Rating of “Satisfied”
A SSRS Rating of “Extremely satisfied”
   2. An improvement at Day 22, 43, 71, 90, 180, 365 or 730 days from baseline (pretreatment “Day 1”) of at least 2 levels in the SSRS Rating of the target thigh.
   3. An improvement at Day 22, 43, 71, 90, 180, 365 or 730 days from baseline (Day 1) of at least 1 level in the SSRS Rating of the target thigh.
   4. In a population of patients with cellulite, the improvement in SSRS Ratings in at least one treatment area was statistically significant compared to placebo wherein the improvement is one or more of Nos. 2 to 3 above.
   5. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   6. At day 180 after the first injection, the improvement seen in the SSRS Rating from baseline was consistent on the right and left thighs.
   7. In a population of patients who have cellulite, the median time to the earliest 2-level SSRS Rating improvement in at least one treatment area is about 50 days, or 60 days, or 70 days, or 80 days, or 90 days.
   8. In a population of patients who have cellulite, the median time to the earliest 1-level SSRS Rating improvement in at least one treatment area is about 15 days, or 20 days, or 30 days, or 40 days, or 50 days, or 60 days, or 70 days, or 80 days, or 90 days.
   9. In a population of patients who have cellulite, the mean subject SSRS Rating separates from placebo 21 days after the first treatment and demonstrate continuous and significant improvement after subsequent treatments.
   10. In a population of patients who have cellulite, the percentage of the subjects who have a 2-level composite response as measured by SSRS Rating in at least one treatment area at Day 71 is from about 1% to 10%, 10% to 20%, 20% to 30%, 30% to 40%, 40% to 50%, or greater than 50%.
   11. In a population of patients who have cellulite, the percentage of the subjects who have a 1-level composite response as measured by SSRS Rating in at least one treatment area at Day 71 is from about 1% to 10%, 10% to 20%, 20% to 30%, 30% to 40%, 40% to 50%, or greater than 50%.
   12. In a population of patients who have cellulite, over one-third, or one-half, or two-thirds, or three-fourths of the patients have at least a 1-level SSRS Rating responses in at least one treatment area by day 71 post-treatment wherein the SSRS Rating results are independent of age, BMI or skin color.
   13. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 13 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 13 above.

In another instance, the injection of about 1 mg to about 20 mg of collagenase according to one of Treatments I to V results in one or more of the results Nos. 1 to 13 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 13 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 13 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 13 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

10. Efficacy as Measured by Subject Satisfaction with Cellulite Treatment (SSCT)

The treatment methods detailed above result in improvements as measured by SSCT. A subject with a response of “Satisfied” or “Very Satisfied” on the SSCT assessment at Day 71 is considered a responder showing efficacy. An improvement for the individual patient at any visit is an improvement of at least 1 level or 1 rating from baseline or any previous score or rating. An improvement for a group of patients at any visit is an improvement of about 0.1 in the mean score or rating from the baseline or any previous mean score or rating. Further, for SSCT, an improvement is at least 0.1 as compared to placebo. In certain embodiments, the treatment methods result in one or more of the following efficacy endpoints as measured by SSCT Rating:

1. In a population of patients with cellulite, the injection of collagenase to at least one treatment area during at least one treatment visit resulted in a statistically significant number of such patients meeting one or more of the following efficacy endpoints:
A SSCT Rating of “Satisfied”
A SSCT Rating of “Very Satisfied”
   2. Increase in the SSCT rating (e.g., 1 to 2, etc) at Day 22, 43, 71, 90, 180, 365 or 730 of the target thigh.
   3. In a population of patients with cellulite, an increase in SSCT Ratings in at least one treatment area was statistically significant compared to placebo wherein the improvement is at Day 22, 43, 71, 90, 180, 365 or 730 in the SSCT Rating of the target thigh.
   4. The treatment resulted in at least 5% of patients maintaining their level of improvement for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   5. At Day 180, the improvement seen in the SSCT Rating is consistent on the right and left thighs.
   6. In a population of patients who have cellulite, the median time to the earliest SSCT Rating improvement in at least one treatment area is about 15 days, or 20 days, or 30 days, or 40 days, or 50 days, or 60 days, or 70 days, or 80 days, or 90 days.
   7. In a population of patients who have cellulite, the mean subject SSCT Ratings separate from placebo 21 days after the first treatment and demonstrates continuous and significant improvement after subsequent treatments.
   8. In a population of patients who have cellulite, the percentage of the subjects who have an improved SSCT Rating in at least one treatment area at Day 71 is from about 1% to 10%, 10% to 20%, 20% to 30%, 30% to 40%, 40% to 50%, or greater than 50%.
   9. In a population of patients who have cellulite, over one-third, or one-half, or two-thirds, or three-fourths of the patients have an improved SSCT Rating in at least one treatment area by Day 71 post-treatment wherein the SSCT Rating results are independent of age, BMI or skin color.
   10. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 10 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 10 above.

In another instance, the injection of about 1 mg to about 20 mg of collagenase according to one of Treatments I to V results in one or more of the results Nos. 1 to 10 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 10 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 10 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 10 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 11. Efficacy as Measured by the Thigh Cellulite Efficacy Scale (CR-TCES; PR-TCES)

An improvement for the individual patient at any visit is an improvement of at least 1 level or 1 rating from baseline or any previous score or rating. An improvement for a group of patients at any visit is an improvement of about 0.1 in the mean score or rating from the baseline or any previous mean score or rating. A responder is any patient showing at least a 20% improvement of maximum total score or rating from baseline. In certain embodiments, the treatment methods detailed above result in one or more of the following efficacy endpoints as measured by CR-TCES and/or PR-TCES:

1. An improvement of at least 0.1 in CR-TCES and/or PR-TCES rating over baseline.
2. An improvement in severity at Day 22, 43, 71, 90, 180, 365 or 730 from baseline (pretreatment “Day 1”) of at least 2 levels of severity in the CR-TCES as assessed live by the clinician of the target thigh.
3. An improvement in severity at Day 22, 43, 71, 90, 180, 365 or 730 from baseline (Day 1) of at least 2 levels of severity in the PR-TCES as assessed by the subject while viewing the digital image of the target thigh.
4. An improvement demonstrated by a 2-level composite response at Day 22, 43, 71, 90, 180, 365 or 730 defined as a subject with an improvement from baseline of at least 2 levels of severity in the CR-TCES and an improvement from baseline of at least 2 levels of severity in the PR-TCES.
5. An improvement in severity at Day 22, 43, 71, 90, 180, 365 or 730 baseline (Day 1) of at least 1 level of severity in the CR-TCES as assessed live by the clinician of the target thigh.
6. An improvement in severity at Day 22, 43, 71, 90, 180, 365 or 730 from baseline (Day 1) of at least 1 level of severity in the PR-TCES as assessed by the subject while viewing the digital image of the target thigh.
7. An improvement demonstrated by a 1-level composite response at Day 22, 43, 71, 90, 180, 365 or 730 defined as a subject with an improvement from baseline of at least 1 level of severity in the CR-TCES and an improvement from baseline of at least 1 level of severity in the PR-TCES.
8. In a population of patients who all had CR-TCES ratings of moderate or severe, the improvement in at least one treatment area was statistically significant compared to placebo wherein the improvement is one or more of Nos. 1 to 7 above.
9. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
10. At Day 180, the improvement seen in the CR-TCES and/or PR-TCES rating from baseline was consistent on the right and left thighs.
11. In a population of patients who all have CR-TCES ratings of moderate or severe, the median time to the earliest 2-level CR-TCES and/or PR-TCES improvement in at least one treatment area is about 50 days, or 60 days, or 70 days, or 80 days, or 90 days.
12. In a population of patients who all have CR-TCES ratings of moderate or severe, the median time to the earliest 1-level CR-TCES and/or PR-TCES improvement in at least one treatment area is about 15 days, or 20 days, or 30 days, or 40 days, or 50 days, or 60 days, or 70 days, or 80 days, or 90 days.
13. In a population of patients who all have CR-TCES ratings of moderate or severe, the mean subject CR-TCES and/or PR-TCES scores separates from placebo 21 days after the first treatment and demonstrate continuous and significant improvement after subsequent treatments.
14. In a population of patients who all have CR-TCES ratings of moderate or severe, the percentage of the subjects who have a 2-level composite response as measured by CR-TCES and/or PR-TCES in at least one treatment area at Day 71 is from about 1% to 10%, 10% to 20%, 20% to 30%, 30% to 40%, 40% to 50%, or greater than 50%.
15. In a population of patients who all have CR-TCES ratings of moderate or severe, the percentage of the subjects who have a 1-level composite response as measured by CR-TCES and/or PR-TCES in at least one treatment area at Day 71 is from about 1% to 10%, 10% to 20%, 20% to 30%, 30% to 40%, 40% to 50%, or greater than 50%.
16. In a population of patients who all have CR-TCES ratings of moderate or severe, over one-third, or one-half, or two-thirds, or three-fourths of the patients have at least a 1-level CR-TCES and/or at least a 1-level PR-TCES responses in at least one treatment area by Day 71 post-treatment wherein the CR-TCES results are independent of age, BMI or skin color.
17. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 17 above.

In another instance, the injection of about 1 mg to about 20 mg of collagenase according to one of Treatments I to V results in one or more of the results Nos. 1 to 17 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 17 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 17 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 12. Efficacy as Measured by Body-O

The treatment methods detailed above result in improved responses as measured by Body-Q. For cellulite, there are 16 scaled items having patient response options ranging from “not at all” to “extremely bothered” over the timeframe of the past week and assuming a Flesch-Kincaid grade reading level. The score ranges from 16 (extremely bothered) to 64 (not at all). For Body-Q total score, a responder is a subject with an increase in the Body-Q total score of at least 16 from baseline at an evaluation time point. For individual Body-Q impact scores, response is an improvement from baseline of at least 1 score interval at each time point. In alternative embodiments, the scaled items may be more or less than 16, and a responder is any patient showing at least a 25% improvement of the maximal total score from baseline.

In certain embodiments, the treatment methods detailed above result in one or more of the following efficacy endpoints as measured by Body-Q:

1. In a population of patients with cellulite, the injection of collagenase to at least one treatment area during at least one treatment visit results in a statistically significant number of such patients meeting one or more of the following efficacy endpoints:
The Body-Q shows improvement across at least one domain selected from the group consisting of:
   An increase in the Body-Q total score of at least 16 from baseline at one or more evaluation time points;
   for individual Body-Q impact scores, an improvement from baseline of at least 1 score interval at each time point; and
   at least a 25% improvement of the maximal total score from baseline.
An increase in the Body-Q total score of at least 16 from baseline at one or more evaluation time points
Body-Q impact scores showing improvement from baseline of at least 1 score interval at one or more evaluation time points
Mean change from baseline in Body-Q appraisal of cellulite at Day 90 and/or Day 180
   2. An improvement in severity at Day 22, 43, 71, 90, 180, 365 or 730 days from baseline (pretreatment “Day 1”) of an increase of at least 16 points in the Body-Q for the target thigh.
   3. In a population of patients with cellulite, a statistically significant improvement in severity over placebo at Day 22, 43, 71, 90, 180, 365 or 730 days baseline (pretreatment Day 1) as indicated by an increase of at least 16 points in the Body-Q for the target thigh.
   4. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose.
   5. At Day 180 after the first injection, the improvement seen in the Body-Q rating from baseline was consistent on the right and left thighs.
   6. In a population of patients who have cellulite, the median time to a Body-Q total score increase of at least 16 from baseline at one or more evaluation time points for at least one treatment area is about 15 days, or 20 days, or 30 days, or 40 days, or 50 days, or 60 days, or 70 days, or 80 days, or 90 days.
   7. In a population of patients who have cellulite, the mean subject Body-Q score separates from placebo 21 days after the first treatment and demonstrate continuous and significant improvement after subsequent treatments.
   8. In a population of patients who have cellulite, over one-third, or over one-half, or over two-thirds, or over three-fourths of the patients have a Body-Q total score increase of at least 16 from baseline at one or more evaluation time points in at least one treatment area by day 71 post-treatment wherein the Body-Q results are independent of age, BMI or skin color.
   9. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 9 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to one of Treatments I to V results in one or more of the results Nos. 1 to 9 above.

In another instance, the injection of about 1 mg to about 20 mg of collagenase according to one of Treatments I to V results in one or more of the results Nos. 1 to 9 above, wherein the collagenase has one or more of the following characteristics:
Vₘₐₓ(min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat}(sec⁻¹) of about to about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,000 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to one of Treatments I to V results in one or more of the results Nos. 1 to 9 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 9 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing one of Treatments I to V and results in one or more of the results Nos. 1 to 9 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 13. Assessment of Treatment Effect as Measured by 3-D Photography/Imagery

As described in the Examples below, 3-D photography or other imagery can be used in the assessment of treatment effect, in particular for the dimple analysis. Dimple analysis (as defined above) using 3-D imagery can include calculations of dimple volume, length, width and area. The calculations may be performed by various known methods such as those described in Eckhouse et al. WO 2018/116304 and WO 2018/116305, Cherry Imaging and those available from Canfield Scientific, Inc. Such measurements of volume, length, width and area may be calculated using digital 3-D greyscale images (with X and Y axis rotation feature) and digital 3-D textured and lit images (with X and Y rotation feature) together with a computer program that analyzes such images. In one embodiment, a buttock treated area, images may be taken of the left treated buttock and/or right treated buttock for each patient before and after treatment. In another embodiment, a thigh treated area, images may be taken of each of the thigh treated areas at 0 degrees, 45 degrees and 90 degrees before and after treatment.

Clinicians and patients may use photographs and imaging tools to assess the severity of cellulite with or without the scales and other tools described herein. In one embodiment, the clinician/investigator or qualified designee photographs each treatment area (each buttock or each thigh) using a supplied standardized 3-D camera in a standardized manner. The subject stands for each photography session and wears a standardized photographic garment. The clinician/investigator or qualified designee photographs each of the 2 treatment areas (2 buttocks or 2 thighs) while the subject is standing in a consistent, standardized relaxed pose. These photographs and the imagery described above may be used in the efficacy analyses described below.

### G. Durability of the Effect

The collagenase treatments described herein have durability in effectiveness as measured by any of the scales or assessment methods disclosed herein. Such durability may range from about 3 months to 5 years or longer. A single injection or series of injections can maintain an improvement in cellulite or continue to improve the appearance of cellulite or reduce the severity of the appearance of cellulite for a long period of time, e.g., about 6 months, 1 year, 2 years, 3 years, 4 years, or 5 years, or longer.

In a certain embodiment, patients receiving collagenase injections continue to exhibit a ≥1-point improvement from baseline for either or both the CR-PCSS and PR-PCS S score at about 6 months post-treatment, or have a ≥1-point improvement from baseline for either or both the CR-PCSS and PR-PCSS score at about 12 months after treatment. Further, such patients have a ≥1-point improvement from baseline for either or both the CR-PCSS and PR-PCSS score at about 22 days, 43 days, 90 days, or 180 days after treatment. For example, at least about 10%, or 15%, or 20%, or 25%, or 30%, or 35%, or 40%, or 45%, or 50%, or 55%, or 60%, or 65%,or 70%, or 75%, or 80%, or 85%, or 90%, or 95%, or 100% of patients demonstrate such durability.

In another aspect, the treatment method evaluates the durability of the effect of 2-level composite responders (patients that had an improvement of at least 2 levels of cellulite severity in both the PR-PCSS and the CR-PCSS), resulting in a statistically significant number demonstrating durability of effect at 6 months and 12 months. In certain embodiments, at least about 10%, or 15%, or 20%, or 25%, or 30%, or 35% or 40%, or 45% or 50%, or 55% or 60% or 65%, or 70%, or 75%, or 80%, or 85%, or 90%, or 95%, or 100% of patients demonstrate such durability.

Non-limiting examples of durability include:
a. The decrease in the CR-PCSS ratings is maintained until Day 180 coupled with the scores on the Subject Satisfaction with Cellulite Treatment Scale support the effectiveness of 3 treatment sessions of CCH 0.84 mg administered as 12 subcutaneous injections per treatment area (×2 treatment areas) to either bilateral buttocks or bilateral thighs.
b. At Day 180, the decrease in the CR-PCSS rating from Baseline (Screening Visit) are consistent on the right and left sides.
c. A 2-level improvement in the CR-PCSS rating in at least 1 treatment area is observed at Day 180. Response is similar for the buttock and thigh regions and for left and right sides.
d. A 1-level improvement in the CR-PCSS rating in at least 1 area is observed at Day 180. Response is similar for the buttock and thigh regions and for left and right sides.
e. The median time to the earliest 2-level CR-PCSS response in at least 1 area is observed at 83 days.
f. The median time to the earliest 1-level CR-PCSS response is 41 days.
g. At Day 180 more than half of patients were either satisfied or very satisfied with treatment.
h. The period of time a patient's score on the Hexsel CSS was first classified as moderate (6-10) or mild (1-5) and continued to be classified as moderate (6-10) or mild (1-5) as compared to her baseline classification as severe (11-15).
i. The period of time measured from the date of a measured improvement to the date when the patient returns to baseline or worse after having demonstrated improvement as measured by one or more of the CR-PCSS, PR-PCSS, Hexsel CSS, Hexsel depression depth score, Likert Scale, dimple analysis, I-GAIS, S-GAIS, PR-CIS, PR-CIS Abbreviated, SSRS, SSCT, CR-TCES, PR-TCES, Body-Q, assessment by photography or other imagery, or any other validated photonumeric or other scale used by clinicians and/or patients to assess cellulite severity, improvement, and/or patient satisfaction.
j. A period of time measured from the date of measured improvement to the date when a subject has an observable loss of response to the treatment as measured by one or more of the CR-PCSS, PR-PCSS, Hexsel CSS, Hexsel depression depth score, Likert Scale, dimple analysis, I-GAIS, S-GAIS, PR-CIS, PR-CIS Abbreviated, SSRS, SSCT, CR-TCES, PR-TCES, Body-Q, assessment by photography or other imagery, or any other validated photonumeric or other scale used by clinicians and/or patients to assess cellulite severity, improvement, and/or patient satisfaction.
k. A period of time measured from the reference time point of response to treatment to when the subject has a change in response (including an improvement over initial response to treatment). Change in response and/or improvement can be measured by one or more of the CR-PCSS, PR-PCSS, Hexsel CSS, Hexsel depression depth score, Likert Scale, dimple analysis, I-GAIS, S-GAIS, PR-CIS, PR-CIS Abbreviated, SSRS, SSCT, CR-TCES, PR-TCES, Body-Q, assessment by photography or other imagery, or any other validated photonumeric or other scale used by clinicians and/or patients to assess cellulite severity, improvement, and/or patient satisfaction. In some embodiments, the reference time point is baseline (pre-dose, Day 1) or Day 71 after treatment.
l. A period of time measured from the visit date that a subject became a 2-level composite responder according to the CR-PCSS and PR-PCSS until the first date of 2 sequential visits at which the assessment ratings return and are sustained to baseline ratings.
m. A period of time measured from the visit date that a subject became a 1-level composite responder according to the CR-PCSS and PR-PCSS until the first date of 2 sequential visits at which the assessment ratings return and are sustained to baseline ratings.

### H. Safety of Collagenase Injections

The studies done to date, some of which are detailed in the Examples below, establish the safety of the treatments described herein. For example, these studies confirm the lack of systemic exposure of collagenase following concurrent, subcutaneous administration of 3.36 mg CCH in four treatment areas. In fact, no new concerns in the safety profile of collagenase were observed with concurrent administration in four treatment areas. The commonly reported events were consistent with the currently known adverse event profile of collagenase.

The majority of subjects treated with CCH experienced at least one TEAE that was mild to moderate in severity. There were no notable differences in the subjects experiencing a TEAE by thigh or buttock treated region. The most common type of TEAEs were injection site reactions, specifically injection site bruising, which did not differ between treatment region (buttock or thigh). Most TEAEs resolving within 21 days. There were no clinically meaningful changes in the clinical and hematology laboratory parameters, urinalysis results, vital signs or concomitant medications. There were no clinically relevant findings in subjects with anti-drug antibodies or neutralizing antibodies.

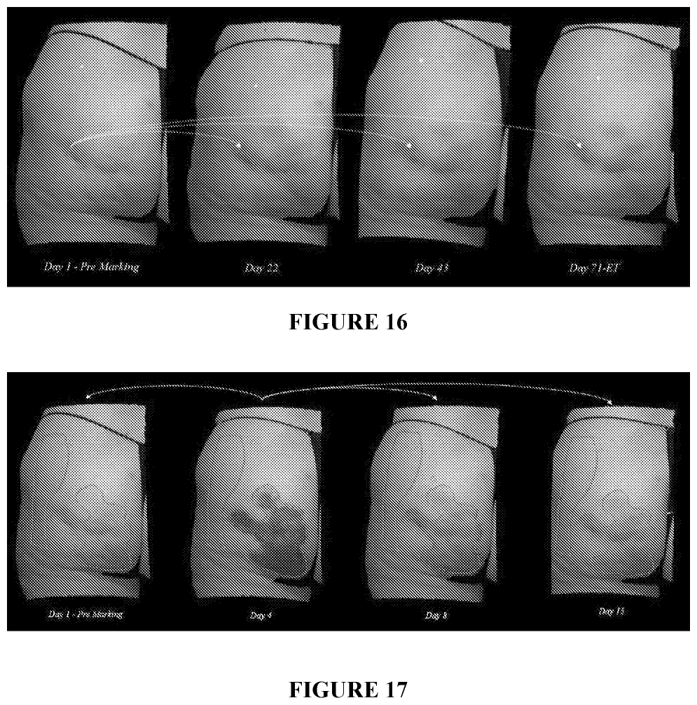

As detailed in the Definitions, Example 1 and FIGS. 16-18, bruising analyses can be performed to measure the extent of bruising over time. In certain embodiments, any bruising caused by the collagenase treatments may resolve or significantly decrease in color intensity at about 3 days, or 4 days, or 5 days, or 6 days, or 7 days, or 8 days, or 9 days, or 10 days, or 11 days, or 12 days, or 13 days, or 14 days, or 15 days, or 20 days after a treatment visit.

### I. Examples

The following examples are included to demonstrate certain embodiments of the present disclosure. Those of skill in the art should, however, in light of the present disclosure, appreciate that modifications can be made in the specific embodiments that are disclosed and still obtain a like or similar result without departing from the spirit and scope of the invention. Therefore, all matter set forth is to be interpreted as illustrative and not in a limiting sense. For instance, in the studies employing CCH, other collagenases can be used in an amount sufficient (therapeutically effective amount) to produce the activity and response comparable to CCH. In the clinical trial results described below, it is to be understood that each numerical value reported is not intended to be strictly limiting. The scope of the present disclosure includes ranges around each value that are consistent with the facts and principles of the inventions described herein. Accordingly, each value may vary up or down by about 1%, 5%, 10%, 15%, 20%, 25%, 30%, 35%, 40%, 45%, 50%, 55%, 60%, 65%, 70%, 75%, 80%, 85%, 90%, 95%, or 100%. In some instances and in keeping with the context and circumstances of a particular value, it may vary up or down by about 125%, 150%, 175%, 200%, 225%, 250%, 275%, or 300%. For example, in one embodiment, the mean percent reduction from baseline in dimple volume in the thigh at day 71 post-treatment is 10%. However, such reduction could be 30%.

### Example 1—Clinical Study Evaluating the Safety and Efficacy of Five Different Injection Techniques of CCH for the Treatment of EFP (Study 209)

An open-label, multicenter study was conducted to evaluate the safety and effectiveness of five different injection techniques when subcutaneously administering CCH for the treatment of adult women with mild, moderate, or severe EFP (as assessed by the investigator using the Clinician Reported Photonumeric Cellulite Severity Scale (CR-PCSS)).

Injection Technique I (Shallow Injection, 3 Aliquots).

In Injection Technique I, CCH was injected subcutaneously using a 1 mL syringe with 0.1 mL gradients while the subject lay in a prone position. The needle was 30 gauge and ½ inch long. Each injection consisted of a single skin injection of CCH administered as three 0.1 mL aliquots to Positions A, B, and C (for a total injection volume of 0.3 mL). As illustrated in FIG. 7, the injection technique used in Treatment I is as follows: (1) position the needle perpendicular to the skin surface and perpendicular to the long axis of a dimple if the dimple is an elongated trough-like dimple, insert the length of the needle through the skin without any downward pressure from the syringe (Position A), and inject 0.1 mL of study drug; (2) withdraw the needle slightly and reposition it at an angle of approximately 450 to the surface of the skin and toward the subject's head, insert the remaining length of the needle without any downward pressure from the syringe (Position B), and inject 0.1 mL of study drug; (3) withdraw the needle slightly and reposition it at an angle of approximately 450 to the surface of the skin and toward the subject's feet, insert the remaining length of the needle without any downward pressure from the syringe (Position C), and inject 0.1 mL of study drug; and (4) withdraw the needle completely from the injection site and proceed to the next injection site. Four 0.9 mL syringes were used to administer 0.3 mL of study drug (3 aliquots of 0.1 mL each) at twelve injection sites in each treatment area during each treatment session.

Injection Technique II (Shallow Injection, 1 Aliquot).

In Injection Technique II, CCH was injected subcutaneously using a 1 mL syringe with 0.1 mL gradients and a 30-gauge ½-inch needle while the subject lay in a prone position. Each injection consisted of a single skin injection of CCH administered as single shallow injection of 0.3 mL study drug. As illustrated in FIG. 8, the injection technique used in Treatment II is as follows: (1) position the needle at an angle of about 300 to the skin surface at the injection and toward the subject's head, insert the length of the needle through the skin, and inject 0.3 mL of study drug; and (2) withdraw the needle completely from the injection site and proceed to the next injection site. Four 0.9 mL syringes were used to administer 0.3 mL of study drug at twelve injection sites in each treatment area during each treatment session.

Injection Technique III (Deep Injection, 1 Aliquot).

In Injection Technique III, CCH was injected subcutaneously using a 1 mL syringe with 0.1 mL gradients and a 30-gauge 1-inch needle while the subject lay in a prone position. Each injection consisted of a single skin injection of CCH administered as single 1-inch injection of 0.3 mL study drug. As illustrated in FIG. 9, the injection technique used in Treatment III is as follows: (1) position the needle at an angle of about 300 to the skin surface at the injection and toward the subject's head, insert the length of the needle through the skin, and inject 0.3 mL of study drug; and (2) withdraw the needle completely from the injection site and proceed to the next injection site. Four syringes containing 0.9 mL of study drug were used to administer 0.3 mL of study drug at twelve injection sites in each treatment area during each treatment session.

Injection Technique IV (Deep and Shallow Injections, 5 Aliquots).

In Injection Technique IV, CCH was injected subcutaneously using a 3 mL syringe with 0.1 mL gradients and a 30-gauge 1-inch needle while the subject lay in a prone position. Each injection consisted of a single skin injection of CCH administered as five 0.3 mL aliquots to Positions A, B, C, D and E (for a total injection volume of 1.5 mL). As illustrated in FIG. 10, the injection technique used in Treatment IV is as follows: (1) position the needle at an angle of about 30° to the skin surface and directed toward the subject's head, insert the length of the needle (1 inch) through the skin (Position A), and inject 0.3 mL of study drug; (2) while maintaining the 30° angle of the needle to the skin surface, withdraw the needle about a half inch and reposition it towards one side of the subject (Position B), and inject 0.3 mL of study drug; (3) while maintaining the 30 angle of the needle to the skin surface, reposition the needle to midway between Positions A and B (Position C, toward the subject's shoulder), and inject 0.3 mL of study drug; (4) while maintaining the 30° angle of the needle to the skin surface, reposition the needle opposition Position B (Position D), and inject 0.3 mL of study drug; (5) while maintaining the 30° angle of the needle to the skin surface, reposition the needle to midway between Positions A and D (Position E, toward the subject's other shoulder), and inject 0.3 mL of study drug; and (6) withdraw the needle completely from the injection site and proceed to the next injection site. Twelve syringes containing 1.5 mL were used to administer 1.5 mL of study drug in 0.3 mL aliquots at twelve injection sites in each treatment area during each treatment session.

Injection Technique V (Shallow Injections, 4 Aliquots).

In Injection Technique V, CCH was injected subcutaneously using a 3 mL syringe with 0.1 mL gradients and a 30-gauge ½-inch needle, while the subject lay in a prone position. Each injection consisted of a single skin injection of CCH administered as four 0.3 mL aliquots to Positions A, B, C and D (for a total injection volume of 1.2 mL). As illustrated in FIG. 11, the injection technique used in Treatment V is as follows: (1) position the needle at an angle of about 30 to the skin surface and directed toward the subject's head, insert the length of the needle (½ inch) through the skin (Position A), and inject 0.3 mL of study drug; (2) while maintaining the 300 angle of the needle to the skin surface, reposition the needle to approximately 600 from Position A and towards the subject's shoulder (Position B), and inject 0.3 mL of study drug; (3) while maintaining the 30 angle of the needle to the skin surface, reposition the needle to approximately 60 from Position B and towards the subject's other shoulder (Position C), and inject 0.3 mL of study drug; (4) while maintaining the 300 angle of the needle to the skin surface, reposition the needle opposite Position A and toward the other side of the subject (Position D), and inject 0.3 mL of study drug; and (5) withdraw the needle completely from the injection site and proceed to the next injection site.

The study drug, CCH, was a sterile lyophilized powder comprising 0.92 mg of collagenase Clostridium histolyticum, sucrose, Tris, mannitol, and hydrochloric acid qs to pH 8.5. CCH sterile diluent for reconstitution was 0.6% sodium chloride and 0.03% calcium chloride dehydrate in water for injection. The diluent was supplied as a terminally-sterilized liquid at 4 mL per vial. CCH was stored at 2-8° C. in a temperature-controlled refrigerator, and the reconstituted CCH solution was administered as soon as practicable after reconstitution.

Study subjects were non-pregnant females 18 years of age or older. Each subject had a score of 2 (mild), 3 (moderate), or 4 (severe) in two thighs or two buttocks at the screening visit, as determined by the investigator using the CR-PCSS scale. Subjects had at least two dimples in each treatment area that were isolated and separated by at least 5 cm from any other dimples, were scored 2 or 3 on the Hexsel depression depth scale, and had a width:length ratio of greater or equal to 0.5. All subjects were deemed to be in good health based upon the results of medical history, physical examination, and laboratory profile at screening. A total of 64 subjects were enrolled in the study. A total of 32 subjects were enrolled in the buttock treatment group, with 31 subjects included in the safety population and 29 subjects included in the evaluable population. One subject in the buttock treatments group was excluded from the safety and evaluable populations because she was not treated. A total of 32 subjects were enrolled in the thigh treatment group, with 32 subjects included in the safety population and 29 subjects included in the evaluable population. Three subjects in the thigh treatment group were excluded from the evaluable population because no Likert scale data was available.

On the Day 1 visit before treatment, the assigned treatment areas (two buttocks or two thighs) were photographed and evaluated for eligibility in the same manner as was done during screening. After confirming the inclusion/exclusion criteria, the investigator selected and marked the dimples in the treatment areas (left and right buttocks, or left and right posterolateral thighs) that were well defined, evident when the subject was standing in a consistent relaxed pose (without the use of any manipulation such as skin pinching or muscle contraction), and suitable for treatment. If both buttocks and both thighs met entry criteria for a single subject, then the thighs were to be treated. No subject was treated for EFP in both the buttocks and the thighs during this study.

The investigator identified at least 2 dimples per treatment area which were spaced out by ≥5 cm from other dimples and scored 2 or 3 on the Hexsel CSS (B) depression depth scale. The dimples in the selected treatment areas were marked for treatment, and the area was photographed again after the dimples were marked.

On Day 1, treatment was assigned sequentially to one of the five injection techniques, and the subject was treated with 24 injections (12 injections per treatment area). At every treatment visit, irrespective of the treatment arm (injection technique) assigned, all eligible subjects were to receive the same amount of CCH (0.84 mg) injected in each assigned treatment area. Subjects returned for up to 2 additional sessions, at which the same procedure was followed (photography, assessment, marking, photography, treatment). Each treatment session was separated by approximately 21 days. All 24 injections for each subject (12 for each treatment area) were required to be within the assigned treatment area for all 3 sessions. Each subject received all 3 treatment sessions. If no injections were given at treatment session 2, the subject still returned for treatment session 3, where the treatment area was again photographed and evaluated. The end of study (EOS) was when the last subject completed the Day 71 end-of-study visit. At the EOS/early termination (ET) visit, each treatment area for each subject was photographed and evaluated, and safety assessments were performed.

The injection volume and concentration for each treatment arm are outlined in Table 20.

TABLE 20
Volume and Concentration of CCH Injections by Injection Type

| | Number of | | Dose/ | | Drug | | Total volume/treatment |
|---|---|---|---|---|---|---|---|
| Treatment | subjects/area | | injection | Volume/injection | concentration/injection | | visit |
| Treatment I: Shallow | 6 | (buttocks) | 0.07 mg | 0.3 mL | 0.23 | mg/mL | 7.2 mL |
| Injection, 3 aliquots | 6 | (thighs) | | (given as 3- 0.1 mL | | | (12 injections/treatment |
| | | | | aliquots) | | | area x 2 treatment areas x |
| | | | | | | | 0.3 mL) |
| Treatment II: Shallow | 6 | (buttocks) | 0.07 mg | 0.3 mL | 0.23 | mg/mL | 7.2 mL |
| injection, 1 aliquot | 6 | (thighs) | | (given as 1- 0.3 mL | | | (12 injections/treatment |
| | | | | aliquot) | | | area x 2 treatment areas x |
| | | | | | | | 0.3 mL) |
| Treatment III: Deep | 6 | (buttocks) | 0.07 mg | 0.3 mL | 0.23 | mg/mL | 7.2 mL |
| injection, 1 aliquot | 6 | (thighs) | | (given as 1- 0.3 mL | | | (12 injections/treatment |
| | | | | aliquot) | | | area x 2 treatment areas x |
| | | | | | | | 0.3 mL) |
| Treatment IV: Deep and | 6 | (buttocks) | 0.07 mg | 1.5 mL | 0.047 | mg/mL | 36 mL |
| shallow injections, | 6 | (thighs) | | (given as 5- 0.3 mL | | | (12 injections/treatment |
| 5 aliquots | | | | aliquots) | | | area x 2 treatment areas x |
| | | | | | | | 1.5 mL) |
| Treatment V: Shallow | 6 | (buttocks) | 0.07 mg | 1.2 mL | 0.060 | mg/mL | 28.8 mL |
| injection, 4 aliquots | 6 | (thighs) | | (given as 4- 0.3 mL | | | (12 injections/treatment |
| | | | | aliquots) | | | area x 2 treatment areas x |
| | | | | | | | 1.2 mL) |

The efficacy endpoints for this study included: (a) the change from baseline in a blinded assessor's Likert Scale score of aesthetic appearance in each treatment area (buttocks or thighs) at Days 22, 43, and 71 by each treatment arm, (b) the change in bruise value of the treatment area compared with normal skin, and (c) the change from baseline in the Hexsel CSS (B) depression depth scale at Days 22, 43, and 71 by treatment arm. Dimple volume was also analyzed using 3-D photography.

Efficacy as Measured by Likert Scale Score.

As shown in the tables below, the Likert Scale score of improvement in aesthetic appearance from baseline was analyzed using a linear model with each injection type (I-V), study visit, interaction of treatment arm and study visit as fixed effects. The fixed effect estimates and 95% confidence intervals (CIs) for interaction effect of treatment arm and study visit are presented. This model was fitted using the response for each treatment area (i.e., left buttock, right buttock, left thigh, and right thigh) and for the average of left and right of treatment area (i.e., average of left buttock and right buttock; average of left thigh and right thigh). Improvement from baseline in aesthetic appearance for each treatment area is assessed by a blind central assessor using a 5-point Likert Scale.

The Likert Scale rating of improvement in aesthetic appearance from baseline was summarized using count and percentage by treatment arm and study visit/day (Day 22, 43, and 71) for each treatment area and with mean and SD. Using the captured images described above, a central assessor completed a 5-point Likert Scale to gauge the level of aesthetic improvement from baseline achieved in each treatment area. Aesthetic improvement was scored as worse (−1), —stayed the same (0), improved (1), much improved (2) and very much improved (3). The efficacy was evaluated at Day 22, 43 and 71. Improvement from baseline in aesthetic appearance for each treatment area (buttocks or thighs) was assessed by a blind central assessor using a 5-point Likert scale at Days 22, 43, and 71 for each treatment arm. Likert scores were as follows: worse (−1), no change (0), improved (1), much improved (2), and very much improved (3). The assessor was provided with 2 images at the same time; baseline was on the left of screen and the visit image was on the right of screen. The assessor evaluated the visit image in comparison with baseline and recorded the Likert score. For the buttock, there was only 1 orientation image per visit to compare with the baseline image. However, for the thigh there were 3 orientation images; thus, there were 3 pairs of images at each visit; 1 pair 0° (lateral), 1 pair 45 (oblique), and 1 pair 90° (posterior). So the assessor had to view all three pairs (i.e., baseline lateral and Day 71 lateral, baseline oblique and Day 71 oblique, and baseline posterior and Day 71 posterior), and then record a single Likert score for the entire thigh (1 value).

Generally, as shown in Tables 21 and 22 below, the left and right buttocks had similar LSMean improvements from baseline in aesthetic appearance based on Likert score for each treatment arm.

TABLE 21
Analysis of Improvement in Aesthetic Appearance from Baseline Likert Scale Rating by Treatment Region

| | | Injection Type | | | | |
|---|---|---|---|---|---|---|
| Visit | | I | II | III | IV | V |
| Treatment Region | Statistic | (N = 12) | (N = 13) | (N = 11) | (N = 12) | (N = 12) |
| Day 22 | | | | | | |
| Average of Left and Right | | | | | | |
| Buttock | N | 6 | 6 | 6 | 6 | 7 |
| | LS Mean | 1.08 | 0.75 | 0.67 | 0.83 | 1.00 |
| | 95% CI | (0.51, 1.65) | (0.18, 1.32) | (0.10, 1.24) | (0.26, 1.40) | (0.47, 1.53) |
| Thigh | N | 5 | 7 | 5 | 6 | 5 |
| | LS Mean | 0.24 | 0.24 | 0.06 | 0.08 | 0.34 |
| | 95% CI | (−0.11, 0.58) | (−0.05, 0.54) | (−0.29, 0.41) | (−0.24, 0.40) | (−0.01, 0.69) |
| Day 43 | | | | | | |
| Average of Left and Right | | | | | | |
| Buttock | n | 6 | 5 | 6 | 6 | 7 |
| | LS Mean | 0.75 | 1.10 | 1.00 | 1.17 | 1.21 |
| | 95% CI | (0.18, 1.32) | (0.47, 1.73) | (0.43, 1.57) | (0.60, 1.74) | (0.69, 1.74) |
| Thigh | n | 5 | 7 | 5 | 6 | 5 |
| | LS Mean | 0.50 | 0.57 | 0.18 | 0.12 | 0.46 |
| | 95% CI | (0.15, 0.85) | (0.28, 0.87) | (−0.17, 0.53) | (−0.20, 0.44) | (0.11, 0.81) |
| Day 71 (EOS) | | | | | | |
| Average of Left and Right | | | | | | |
| Buttock | n | 6 | 6 | 6 | 6 | 7 |
| | LS Mean | 1.08 | 1.08 | 1.00 | 0.50 | 1.29 |
| | 95% CI | (0.51, 1.65) | (0.51, 1.65) | (0.43, 1.57) | (−0.07, 1.07) | (0.76, 1.81) |
| Thigh | n | 6 | 7 | 4 | 5 | 5 |
| | LS Mean | 0.38 | 0.40 | 0.18 | 0.20 | 0.40 |
| | 95% CI | (0.06, 0.70) | (0.10, 0.70) | (−0.22, 0.57) | (−0.15, 0.55) | (0.05, 0.75) |

Note:
EOS = End of Study.

As also can be seen in Table 21, averaging the left and right buttocks, at Day 71, all injection types showed an improvement in aesthetic appearance compared with baseline, with injection types I, II, III, and V having a significant improvement; injection type IV had a significant improvement at Day 22 and Day 43 only. In general, the LSMean improvement in aesthetic appearance of the buttocks compared with baseline tended to improve from Day 22 to Day 71, except for injection type IV. Half of the subjects in injection type IV had no change or a worse Likert score at Day 71 compared with baseline in the left and right buttock. Generally, the left and right thighs had similar LSMean improvements from baseline in aesthetic appearance for each treatment arm. Averaging the left and right thighs, at Day 71, all injection types showed an improvement in aesthetic appearance compared with baseline, with injection type I having a significant improvement. In general, the LSMean improvement in aesthetic appearance of the thighs compared with baseline tended to decline from Day 22 to Day 71 in all injection types. While in the right and left thigh some subjects had a worse Likert score at Day 71 compared with baseline, applicant believes that the method of using 3 pairs to generate 1 entire thigh score may be flawed.

Further efficacy results from the Likert Scale Rating data are summarized in Tables 22 and 23 below.

TABLE 22
Analysis of improvement in aesthetic appearance from
baseline Likert scale rating by treatment area.

| Visit | | Injection Type | | | | |
|---|---|---|---|---|---|---|
| Treatment Area | Statistic | I | II | III | IV | V |
| Day 22 | | | | | | |
| Left buttock | n | 6 | 6 | 6 | 6 | 7 |
| | LS mean | 0.83 | 1.00 | 0.67 | 0.67 | 1.29 |
| | 95% CI | (0.11, 1.55) | (0.28, 1.72) | (−0.05, 1.39) | (−0.05, 1.39) | (0.62, 1.95) |
| Right buttock | n | 6 | 6 | 6 | 6 | 7 |
| | LS mean | 1.33 | 0.50 | 0.67 | 1.00 | 0.71 |
| | 95% CI | (0.66, 2.01) | (−0.18, 1.18) | (−0.01, 1.34) | (0.32, 1.68) | (0.09, 1.34) |
| Left thigh | n | 5 | 7 | 5 | 6 | 5 |
| lateral | | | | | | |
| | LS mean | 0.20 | 0.71 | <0.01 | <0.01 | 0.40 |
| | 95% CI | (−0.37, 0.77) | (0.23, 1.20) | (−0.57, 0.57) | (−0.52, 0.52) | (−0.17, 0.97) |
| Left thigh | n | 5 | 7 | 5 | 6 | 5 |
| oblique | | | | | | |
| | LS mean | 0.20 | 0.29 | 0.00 | 0.17 | 0.40 |
| | 95% CI | (−0.38, 0.78) | (−0.20, 0.77) | (−0.58, 0.58) | (−0.36, 0.69) | (−0.18, 0.98) |
| Left thigh | n | 5 | 7 | 5 | 6 | 5 |
| posterior | | | | | | |
| | LS mean | 0.40 | 0.43 | 0.20 | 0.17 | 0.40 |
| | 95% CI | (−0.17, 0.97) | (−0.05, 0.91) | (−0.37, 0.37) | (−0.35, 0.69) | (−0.17, 0.97) |
| Right thigh | n | 5 | 7 | 5 | 6 | 5 |
| lateral | | | | | | |
| | LS mean | <0.01 | 0.29 | 0.00 | <−0.01 | 0.00 |
| | 95% CI | (−0.37, 0.37) | (−0.02, 0.60) | (−0.37, 0.37) | (−0.34, 0.34) | (−0.37, 0.37) |
| Right thigh | n | 5 | 7 | 5 | 6 | 5 |
| oblique | | | | | | |
| | LS mean | <0.01 | <0.01 | <0.01 | <0.01 | 0.40 |
| | 95% CI | (−0.56, 0.56) | (−0.47, 0.47) | (−0.56, 0.56) | (−0.51, 0.51) | (−0.16, 0.96) |
| Right thigh | n | 5 | 7 | 5 | 6 | 5 |
| posterior | | | | | | |
| | LS mean | 0.60 | −0.29 | 0.20 | 0.17 | 0.40 |
| | 95% CI | (0.00, 1.20) | (−0.79, 0.22) | (−0.40, 0.80) | (−0.38, 0.71) | (−0.16, 0.96) |
| Day 43 | | | | | | |
| Left buttock | n | 6 | 5 | 6 | 6 | 7 |
| | LS mean | 0.83 | 1.00 | 1.17 | 1.00 | 1.43 |
| | 95% CI | (0.11, 1.55) | (0.21, 1.79) | (0.45, 1.89) | (0.28, 1.72) | (0.76, 2.10) |
| Right buttock | n | 6 | 5 | 6 | 6 | 7 |
| | LS mean | 0.67 | 1.20 | 0.83 | 1.33 | 1.00 |
| | 95% CI | (−0.01, 1.34) | (0.46, 1.94) | (0.16, 1.51) | (0.66, 2.01) | (0.37, 1.63) |
| Left thigh | n | 5 | 7 | 5 | 6 | 5 |
| lateral | | | | | | |
| | LS mean | 0.20 | 0.57 | 0.40 | 0.33 | 0.60 |
| | 95% CI | (−0.37, 0.77) | (0.09, 1.05) | (−0.17, 0.97) | (−0.19, 0.85) | (0.03, 1.17) |
| Left thigh | n | 5 | 7 | 5 | 6 | 5 |
| oblique | | | | | | |
| | LS mean | 0.80 | 0.57 | <−0.01 | 0.17 | 0.80 |
| | 95% CI | (0.22, 1.38) | (0.08, 1.06) | (−0.58, 0.58) | (−0.36, 0.69) | (0.22, 1.38) |
| Left thigh | n | 5 | 7 | 5 | 6 | 5 |
| posterior | | | | | | |
| | LS mean | 0.60 | 0.71 | <−0.010.20 | 0.17 | −0.20 |
| | 95% CI | (0.03, 1.17) | (0.23, 1.20) | (−0.57, 0.57) | (−0.35, 0.69) | (−0.77, 0.37) |
| Right thigh | n | 5 | 7 | 5 | 6 | 5 |
| lateral | | | | | | |
| | LS mean | 0.20 | 0.29 | 0.20 | −0.17 | <0.01 |
| | 95% CI | (−0.17, 0.57) | (−0.02, 0.60) | (−0.17, 0.57) | (−0.50, 0.17) | (−0.37, 0.37) |
| Right thigh | n | 5 | 7 | 5 | 6 | 5 |
| oblique | | | | | | |
| | LS mean | 0.60 | 0.86 | −0.20 | 0.17 | 1.00 |
| | 95% CI | (0.04, 1.16) | (0.38, 1.33) | (−0.76, 0.36) | (−0.34, 0.68) | (0.44, 1.56) |
| Right thigh | n | 5 | 7 | 5 | 6 | 5 |
| posterior | | | | | | |
| | LS mean | 0.60 | 0.43 | 0.60 | <−0.01 | 0.60 |
| | 95% CI | (0.00, 1.20) | (−0.08, 0.93) | (0.00, 1.20) | (−0.54, 0.54) | (0.00, 1.20) |
| Day 71 (EOS) | | | | | | |
| Left buttock | n | 6 | 6 | 6 | 6 | 7 |
| | LS mean | 1.17 | 1.17 | 1.33 | 0.50 | 1.43 |
| | 95% CI | (0.45, 1.89) | (0.45, 1.89) | (0.61, 2.05) | (−0.22, 1.22) | (0.76, 2.10) |
| Right buttock | n | 6 | 6 | 6 | 6 | 7 |
| | LS mean | 1.00 | 1.00 | 0.67 | 0.50 | 1.14 |
| | 95% CI | (0.32, 1.68) | (0.32, 1.68) | (−0.01, 1.34) | (−0.18, 1.18) | (0.52, 1.77) |
| Left thigh | n | 6 | 7 | 4 | 5 | 5 |
| lateral | | | | | | |
| | LS mean | 0.33 | 0.86 | 0.25 | <0.01 | 0.40 |
| | 95% CI | (−0.19, 0.85) | (0.37, 1.34) | (−0.39, 0.89) | (−0.57, 0.57) | (−0.17, 0.97) |
| Left thigh | n | 6 | 7 | 4 | 5 | 5 |
| oblique | | | | | | |
| | LS mean | 0.67 | 0.71 | 0.25 | 0.40 | 0.40 |
| | 95% CI | (0.14, 1.19) | (0.23, 1.20) | (−0.39, 0.89) | (−0.18, 0.98) | (−0.18, 0.98) |
| Left thigh | n | 6 | 7 | 4 | 5 | 5 |
| posterior | | | | | | |
| | LS mean | 0.33 | 0.14 | <−0.01 | 0.20 | −0.20 |
| | 95% CI | (−0.19, 0.85) | (−0.34, 0.63) | (−0.64, 0.64) | (−0.37, 0.77) | (−0.77, 0.37) |
| Right thigh | n | 6 | 7 | 4 | 5 | 5 |
| lateral | | | | | | |
| | LS mean | 0.17 | 0.29 | 0.25 | <0.01 | 0.60 |
| | 95% CI | (−0.17, 0.50) | (−0.02, 0.60) | (−0.16, 0.66) | (−0.37, 0.37) | (0.23, 0.97) |
| Right thigh | n | 6 | 7 | 4 | 5 | 5 |
| oblique | | | | | | |
| | LS mean | 0.33 | 0.29 | 0.25 | 0.60 | 0.60 |
| | 95% CI | (−0.18, 0.84) | (−0.19, 0.76) | (−0.38, 0.88) | (0.04, 1.16) | (0.04, 1.16) |
| Right thigh | n | 6 | 7 | 4 | 5 | 5 |
| posterior | | | | | | |
| | LS mean | 0.50 | 0.14 | <0.01 | <0.01 | 0.60 |
| | 95% CI | (−0.04, 1.04) | (−0.36, 0.65) | (−0.67, 0.67) | (−0.60, 0.60) | (0.00, 1.20) |

TABLE 23
Summary of improvement in aesthetic appearance from
baseline Likert scale rating by treatment area.

| | | Injection type | | | | | | | |
|---|---|---|---|---|---|---|---|---|---|
| | Statistic | I | II | III | | IV | | V | |
| Left Buttock | n | 6 | 6 | 6 | | 6 | | 7 | |
| Day 22 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 2 (33.3) | 1 (16.7) | 2 | (33.3) | 3 | (50.0) | 2 | (28.6) |
| 1 (Improved) | n (%) | 3 (50.0) | 4 (66.7) | 4 | (66.7) | 2 | (33.3) | 2 | (28.6) |
| 2 (Much | n (%) | 1 (16.7) | 1 (16.7) | 0 | | 1 | (16.7) | 2 | (28.6) |
| improved) | | | | | | | | | |
| 3 (Very much | n (%) | 0 | 0 | 0 | | 0 | | 1 | (14.3) |
| improved) | | | | | | | | | |
| | n | 6 | 6 | 6 | | 6 | | 7 | |
| | Mean | 0.8 | 1.0 | 0.7 | | 0.7 | | 1.3 | |
| | Std | 0.75 | 0.63 | 0.52 | | 0.82 | | 1.11 | |
| | Median | 1.0 | 1.0 | 1.0 | | 0.5 | | 1.0 | |
| | Min | 0 | 0 | 0 | | 0 | | 0 | |
| | Max | 2 | 2 | 1 | | 2 | | 3 | |
| Left Buttock | n | 6 | 6 | 6 | | 6 | | 7 | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 2 (33.3) | 1 (16.7) | 2 | (33.3) | 2 | (33.3) | 2 | (28.6) |
| 1 (Improved) | n (%) | 3 (50.0) | 3 (50.0) | 1 | (16.7) | 2 | (33.3) | 1 | (14.3) |
| 2 (Much | n (%) | 1 (16.7) | 1 (16.7) | 3 | (50.0) | 2 | (33.3) | 3 | (42.9) |
| improved) | | | | | | | | | |
| 3 (Very much | n (%) | 0 | 0 | 0 | | 0 | | 1 | (14.3) |
| improved) | | | | | | | | | |
| | n | 6 | 5 | 6 | | 6 | | 7 | |
| | Mean | 0.8 | 1.0 | 1.2 | | 1.0 | | 1.4 | |
| | Std | 0.75 | 0.71 | 0.98 | | 0.89 | | 1.13 | |
| | Median | 1.0 | 1.0 | 1.5 | | 1.0 | | 2.0 | |
| | Min | 0 | 0 | 0 | | 0 | | 0 | |
| | Max | 2 | 2 | 2 | | 2 | | 3 | |
| Left Buttock | n | 6 | 6 | 6 | | 6 | | 7 | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 0 | 0 | | 1 | (16.7) | 0 | |
| 0 (No change) | n (%) | 1 (16.7) | 0 | 1 | (16.7) | 2 | (33.3) | 2 | (28.6) |
| 1 (Improved) | n (%) | 3 (50.0) | 5 (83.3) | 2 | (33.3) | 2 | (33.3) | 2 | (28.6) |
| 2 (Much | n (%) | 2 (33.3) | 1 (16.7) | 3 | (50.0) | 1 | (16.7) | 1 | (14.3) |
| improved) | | | | | | | | | |
| 3 (Very much | n (%) | 0 | 0 | 0 | | 0 | | 2 | (28.6) |
| improved) | | | | | | | | | |
| | n | 6 | 6 | 6 | | 6 | | 7 | |
| | Mean | 1.2 | 1.2 | 1.3 | | 0.5 | | 1.4 | |
| | Std | 0.75 | 0.41 | 0.82 | | 1.05 | | 1.27 | |
| | Median | 1.0 | 1.0 | 1.5 | | 0.5 | | 1.0 | |
| | Min | 0 | 1 | 0 | | −1 | | 0 | |
| | Max | 2 | 2 | 2 | | 2 | | 3 | |
| Right Buttock | n | 6 | 6 | 6 | | 6 | | 7 | |
| Day 22 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 0 | 4 (66.7) | 3 | (50.0) | 2 | (33.3) | 2 | (28.6) |
| 1 (Improved) | n (%) | 4 (66.7) | 1 (16.7) | 2 | (33.3) | 2 | (33.3) | 5 | (71.4) |
| 2 (Much | n (%) | 2 (33.3) | 1 (16.7) | 1 | (16.7) | 2 | (33.3) | 0 | |
| improved) | | | | | | | | | |
| 3 (Very much | n (%) | 0 | 0 | 0 | | 0 | | 0 | |
| improved) | | | | | | | | | |
| | n | 6 | 6 | 6 | | 6 | | 7 | |
| | Mean | 1.3 | 0.5 | 0.7 | | 1.0 | | 0.7 | |
| | Std | 0.52 | 0.84 | 0.82 | | 0.89 | | 0.49 | |
| | Median | 1.0 | 0.0 | 0.5 | | 1.0 | | 1.0 | |
| | Min | 1 | 0 | 0 | | 0 | | 0 | |
| | Max | 2 | 2 | 2 | | 2 | | 1 | |
| Right Buttock | n | 6 | 6 | 6 | | 6 | | 7 | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 2 (33.3) | 2 (33.3) | 3 | (50.0) | 1 | (16.7) | 2 | (28.6) |
| 1 (Improved) | n (%) | 4 (66.7) | 1 (16.7) | 1 | (16.7) | 3 | (50.0) | 3 | (42.9) |
| 2 (Much | n (%) | 0 | 1 (16.7) | 2 | (33.3) | 1 | (16.7) | 2 | (28.6) |
| improved) | | | | | | | | | |
| 3 (Very much | n (%) | 0 | 1 (16.7) | 0 | | 1 | (16.7) | 0 | |
| improved) | | | | | | | | | |
| | n | 6 | 5 | 6 | | 6 | | 7 | |
| | Mean | 0.7 | 1.2 | 0.8 | | 1.3 | | 1.0 | |
| | Std | 0.52 | 1.30 | 0.98 | | 1.03 | | 0.82 | |
| | Median | 1.0 | 1.0 | 0.5 | | 1.0 | | 1.0 | |
| | Min | 0 | 0 | 0 | | 0 | | 0 | |
| | Max | 1 | 3 | 2 | | 3 | | 2 | |
| Right Buttock | n | 6 | 6 | 6 | | 6 | | 7 | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 0 | 0 | | 1 | (16.7) | 0 | |
| 0 (No change) | n (%) | 1 (16.7) | 2 (33.3) | 3 | (50.0) | 2 | (33.3) | 1 | (14.3) |
| 1 (Improved) | n (%) | 4 (66.7) | 2 (33.3) | 2 | (33.3) | 2 | (33.3) | 4 | (57.1) |
| 2 (Much | n (%) | 1 (16.7) | 2 (33.3) | 1 | (16.7) | 1 | (16.7) | 2 | (28.6) |
| improved) | | | | | | | | | |
| 3 (Very much | n (%) | 0 | 0 | 0 | | 0 | | 0 | |
| improved) | | | | | | | | | |
| | n | 6 | 6 | 6 | | 6 | | 7 | |
| | Mean | 1.0 | 1.0 | 0.7 | | 0.5 | | 1.1 | |
| | Std | 0.63 | 0.89 | 0.82 | | 1.05 | | 0.69 | |
| | Median | 1.0 | 1.0 | 0.5 | | 0.5 | | 1.0 | |
| | Min | 0 | 0 | 0 | | −1 | | 0 | |
| | Max | 2 | 2 | 2 | | 2 | | 2 | |
| Left Thigh | n | 6 | 7 | 5 | | 6 | | 5 | |
| Lateral | | | | | | | | | |
| Day 22 | | | | | | | | | |
| Likert Scale | | | | | | | | | |
| Rating of | | | | | | | | | |
| improvement | | | | | | | | | |
| −1 (Worse) | n (%) | 0 | 1 (14.3) | 0 | | 0 | | 0 | |
| 0 (No change) | n (%) | 4 (66.7) | 1 (14.3) | 5 | (100) | 6 | (100) | 3 | (60.0) |
| 1 (Improved) | n (%) | 1 (16.7) | 4 (57.1) | 0 | | 0 | | 2 | (40.0) |
| 2 (Much | n (%) | 0 | 1 (14.3) | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.2 | 0.7 | 0.0 | | 0.0 | | 0.4 | |
| | Std | 0.45 | 0.95 | 0.00 | | 0.00 | | 0.55 | |
| | Median | 0.0 | 1.0 | 0.0 | | 0.0 | | 0.0 | |
| | Min | 0 | −1 | 0 | | 0 | | 0 | |
| | Max | 1 | 2 | 0 | | 0 | | 1 | |
| Left Thigh | n | 6 | 7 | 5 | | 6 | | 5 | |
| Lateral | | | | | | | | | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 4 (66.7) | 4 (57.1) | 3 | (60.0) | 5 | (83.3) | 3 | (60.0) |
| 1 (Improved) | n (%) | 1 (16.7) | 2 (28.6) | 2 | (40.0) | 0 | | 1 | (20.0) |
| 2 (Much | n (%) | 0 | 1 (14.3) | 0 | | 1 | (16.7) | 1 | (20.0) |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.2 | 0.6 | 0.4 | | 0.3 | | 0.6 | |
| | Std | 0.45 | 0.79 | 0.55 | | 0.82 | | 0.89 | |
| | Median | 0.0 | 0.0 | 0.0 | | 0.0 | | 0.0 | |
| | Min | 0 | 0 | 0 | | 0 | | 0 | |
| | Max | 1 | 2 | 1 | | 2 | | 2 | |
| Left Thigh | n | 6 | 7 | 5 | | 6 | | 5 | |
| Lateral | | | | | | | | | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 1 | (20.0) |
| 0 (No change) | n (%) | 4 (66.7) | 2 (28.6) | 3 | (60.0) | 5 | (83.3) | 1 | (20.0) |
| 1 (Improved) | n (%) | 2 (33.3) | 4 (57.1) | 1 | (20.0) | 0 | | 3 | (60.0) |
| 2 (Much | n (%) | 0 | 1 (14.3) | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 6 | 7 | 4 | | 5 | | 5 | |
| | Mean | 0.3 | 0.9 | 0.3 | | 0.0 | | 0.4 | |
| | Std | 0.52 | 0.69 | 0.50 | | 0.00 | | 0.89 | |
| | Median | 0.0 | 1.0 | 0.0 | | 0.0 | | 1.0 | |
| | Min | 0 | 0 | 0 | | 0 | | −1 | |
| | Max | 1 | 2 | 1 | | 0 | | 1 | |
| Left Thigh | n | 6 | 7 | 5 | | 6 | | 5 | |
| Oblique | | | | | | | | | |
| Day 22 | | | | | | | | | |
| Likert Scale | | | | | | | | | |
| Rating of | | | | | | | | | |
| improvement | | | | | | | | | |
| −1 (Worse) | n (%) | 0 | 1 (14.3) | 0 | | 0 | | 0 | |
| 0 (No change) | n (%) | 4 (66.7) | 3 (42.9) | 5 | (100) | 5 | (83.3) | 3 | (60.0) |
| 1 (Improved) | n (%) | 1 (16.7) | 3 (42.9) | 0 | | 1 | (16.7) | 2 | (40.0) |
| 2 (Much | n (%) | 0 | 0 | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.2 | 0.3 | 0.0 | | 0.2 | | 0.4 | |
| | Std | 0.45 | 0.76 | 0.00 | | 0.41 | | 0.55 | |
| | Median | 0.0 | 0.0 | 0.0 | | 0.0 | | 0.0 | |
| | Min | 0 | −1 | 0 | | 0 | | 0 | |
| | Max | 1 | 1 | 0 | | 1 | | 1 | |
| Left Thigh | n | 6 | 7 | 5 | | 6 | | 5 | |
| Oblique | | | | | | | | | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 1 (16.7) | 5 (71.4) | 5 | (100) | 5 | (83.3) | 1 | (20.0) |
| 1 (Improved) | n (%) | 4 (66.7) | 0 | 0 | | 1 | (16.7) | 4 | (80.0) |
| 2 (Much | n (%) | 0 | 2 (28.6) | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.8 | 0.6 | 0.0 | | 0.2 | | 0.8 | |
| | Std | 0.45 | 0.98 | 0.00 | | 0.41 | | 0.45 | |
| | Median | 1.0 | 0.0 | 0.0 | | 0.0 | | 1.0 | |
| | Min | 0 | 0 | 0 | | 0 | | 0 | |
| | Max | 1 | 2 | 0 | | 1 | | 1 | |
| Left Thigh | n | 6 | 7 | 5 | | 6 | | 5 | |
| Oblique | | | | | | | | | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 1 | (20.0) |
| 0 (No change) | n (%) | 4 (66.7) | 3 (42.9) | 3 | (60.0) | 3 | (50.0) | 1 | (20.0) |
| 1 (Improved) | n (%) | 0 | 3 (42.9) | 1 | (20.0) | 2 | (33.3) | 3 | (60.0) |
| 2 (Much | n (%) | 2 (33.3) | 1 (14.3) | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 6 | 7 | 4 | | 5 | | 5 | |
| | Mean | 0.7 | 0.7 | 0.3 | | 0.4 | | 0.4 | |
| | Std | 1.03 | 0.76 | 0.50 | | 0.55 | | 0.89 | |
| | Median | 0.0 | 1.0 | 0.0 | | 0.0 | | 1.0 | |
| | Min | 0 | 0 | 0 | | 0 | | −1 | |
| | Max | 2 | 2 | 1 | | 1 | | 1 | |
| Left Thigh | n | 6 | 7 | 5 | | 6 | | 5 | |
| Posterior | | | | | | | | | |
| Day 22 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 3 (50.0) | 4 (57.1) | 4 | (80.0) | 5 | (83.3) | 3 | (60.0) |
| 1 (Improved) | n (%) | 2 (33.3) | 3 (42.9) | 1 | (20.0) | 1 | (16.7) | 2 | (40.0) |
| 2 (Much | n %) | 0 | 0 | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.4 | 0.4 | 0.2 | | 0.2 | | 0.4 | |
| | Std | 0.55 | 0.53 | 0.45 | | 0.41 | | 0.55 | |
| | Median | 0.0 | 0.0 | 0.0 | | 0.0 | | 0.0 | |
| | Min | 0 | 0 | 0 | | 0 | | 0 | |
| | Max | 1 | 1 | 1 | | 1 | | 1 | |
| Left Thigh | n | 6 | 7 | 5 | | 6 | | 5 | |
| Posterior | | | | | | | | | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 1 | (16.7) | 2 | (40.0) |
| 0 (No change) | n (%) | 2 (33.3) | 3 (42.9) | 5 | (100) | 3 | (50.0) | 2 | (40.0) |
| 1 (Improved) | n (%) | 3 (50.0) | 3 (42.9) | 0 | | 2 | (33.3) | 1 | (20.0) |
| 2 (Much | n (%) | 0 | 1 (14.3) | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.6 | 0.7 | 0.0 | | 0.2 | | −0.2 | |
| | Std | 0.55 | 0.76 | 0.00 | | 0.75 | | 0.84 | |
| | Median | 1.0 | 1.0 | 0.0 | | 0.0 | | 0.0 | |
| | Min | 0 | 0 | 0 | | −1 | | −1 | |
| | Max | 1 | 2 | 0 | | 1 | | 1 | |
| Left Thigh | n | 6 | 7 | 5 | | 6 | | 5 | |
| Posterior | | | | | | | | | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 2 (28.6) | 0 | | 0 | | 2 | (40.0) |
| 0 (No change) | n (%) | 4 (66.7) | 3 (42.9) | 4 | (80.0) | 4 | (66.7) | 2 | (40.0) |
| 1 (Improved) | n (%) | 2 (33.3) | 1 (14.3) | 0 | | 1 | (16.7) | 1 | (20.0) |
| 2 (Much | n (%) | 0 | 1 (14.3) | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 6 | 7 | 4 | | 5 | | 5 | |
| | Mean | 0.3 | 0.1 | 0.0 | | 0.2 | | −0.2 | |
| | Std | 0.52 | 1.07 | 0.00 | | 0.45 | | 0.84 | |
| | Median | 0.0 | 0.0 | 0.0 | | 0.0 | | 0.0 | |
| | Min | 0 | −1 | 0 | | 0 | | −1 | |
| | Max | 1 | 2 | 0 | | 1 | | 1 | |
| Right Thigh | n | 6 | 7 | 5 | | 6 | | 5 | |
| Lateral | | | | | | | | | |
| Day 22 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 5 (83.3) | 5 (71.4) | 5 | (100) | 6 | (100) | 5 | (100) |
| 1 (Improved) | n (%) | 0 | 2 (28.6) | 0 | | 0 | | 0 | |
| 2 (Much | n (%) | 0 | 0 | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.0 | 0.3 | 0.0 | | 0.0 | | 0.0 | |
| | Std | 0.00 | 0.49 | 0.00 | | 0.00 | | 0.00 | |
| | Median | 0.0 | 0.0 | 0.0 | | 0.0 | | 0.0 | |
| | Min | 0 | 0 | 0 | | 0 | | 0 | |
| | Max | 0 | 1 | 0 | | 0 | | 0 | |
| Right Thigh | n | 6 | 7 | 5 | | 6 | | 5 | |
| Lateral | | | | | | | | | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 1 | (16.7) | 0 | |
| 0 (No change) | n (%) | 4 (66.7) | 5 (71.4) | 4 | (80.0) | 5 | (83.3) | 5 | (100) |
| 1 (Improved) | n (%) | 1 (16.7) | 2 (28.6) | 1 | (20.0) | 0 | | 0 | |
| 2 (Much | n (%) | 0 | 0 | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.2 | 0.3 | 0.2 | | −0.2 | | 0.0 | |
| | Std | 0.45 | 0.49 | 0.45 | | 0.41 | | 0.00 | |
| | Median | 0.0 | 0.0 | 0.0 | | 0.0 | | 0.0 | |
| | Min | 0 | 0 | 0 | | −1 | | 0 | |
| | Max | 1 | 1 | 1 | | 0 | | 0 | |
| Right Thigh | n | 6 | 7 | 5 | | 6 | | 5 | |
| Lateral | | | | | | | | | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 0 | 0 | | 0 | | 0 | |
| 0 (No change) | n (%) | 5 (83.3) | 5 (71.4) | 3 | (60.0) | 5 | (83.3) | 3 | (60.0) |
| 1 (Improved) | n (%) | 1 (16.7) | 2 (28.6) | 1 | (20.0) | 0 | | 1 | (20.0) |
| 2 (Much | n (%) | 0 | 0 | 0 | | 0 | | 1 | (20.0) |
| improved) | | | | | | | | | |
| 3 (Very much | n (%) | 0 | 0 | 0 | | 0 | | 0 | |
| improved) | | | | | | | | | |
| | n | 6 | 7 | 4 | | 5 | | 5 | |
| | Mean | 0.2 | 0.3 | 0.3 | | 0.0 | | 0.6 | |
| | Std | 0.41 | 0.49 | 0.50 | | 0.00 | | 0.89 | |
| | Median | 0.0 | 0.0 | 0.0 | | 0.0 | | 0.0 | |
| | Min | 0 | 0 | 0 | | 0 | | 0 | |
| | Max | 1 | 1 | 1 | | 0 | | 2 | |
| Right Thigh | n | 6 | 7 | 5 | | 6 | | 5 | |
| Oblique | | | | | | | | | |
| Day 22 | | | | | | | | | |
| Likert Scale | | | | | | | | | |
| Rating of | | | | | | | | | |
| improvement | | | | | | | | | |
| −1 (Worse) | n (%) | 0 | 2 (28.6) | 1 | (20.0) | 0 | | 0 | |
| 0 (No change) | n (%) | 5 (83.3) | 3 (42.9) | 3 | (60.0) | 6 | (100) | 3 | (60.0) |
| 1 (Improved) | n (%) | 0 | 2 (28.6) | 1 | (20.0) | 0 | | 2 | (40.0) |
| 2 (Much | n (%) | 0 | 0 | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.0 | 0.0 | 0.0 | | 0.0 | | 0.4 | |
| | Std | 0.00 | 0.82 | 0.71 | | 0.00 | | 0.55 | |
| | Median | 0.0 | 0.0 | 0.0 | | 0.0 | | 0.0 | |
| | Min | 0 | −1 | −1 | | 0 | | 0 | |
| | Max | 0 | 1 | 1 | | 0 | | 1 | |
| Right Thigh | n | 6 | 7 | 5 | | 6 | | 5 | |
| Oblique | | | | | | | | | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 1 | (20.0) | 0 | | 0 | |
| 0 (No change) | n (%) | 2 (33.3) | 3 (42.9) | 4 | (80.0) | 5 | (83.3) | 1 | (20.0) |
| 1 (Improved) | n (%) | 3 (50.0) | 2 (28.6) | 0 | | 1 | (16.7) | 3 | (60.0) |
| 2 (Much | n (%) | 0 | 2 (28.6) | 0 | | 0 | | 1 | (20.0) |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.6 | 0.9 | −0.2 | | 0.2 | | 1.0 | |
| | Std | 0.55 | 0.90 | 0.45 | | 0.41 | | 0.71 | |
| | Median | 1.0 | 1.0 | 0.0 | | 0.0 | | 1.0 | |
| | Min | 0 | 0 | −1 | | 0 | | 0 | |
| | Max | 1 | 2 | 0 | | 1 | | 2 | |
| Right Thigh | n | 6 | 7 | 5 | | 6 | | 5 | |
| Oblique | | | | | | | | | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 1 (14.3) | 0 | | 0 | | 0 | |
| 0 (No change) | n (%) | 4 (66.7) | 3 (42.9) | 3 | (60.0) | 3 | (50.0) | 2 | (40.0) |
| 1 (Improved) | n (%) | 2 (33.3) | 3 (42.9) | 1 | (20.0) | 1 | (16.7) | 3 | (60.0) |
| 2 (Much | n (%) | 0 | 0 | 0 | | 1 | (16.7) | 0 | |
| improved) | | | | | | | | | |
| 3 (Very much | n (%) | 0 | 0 | 0 | | 0 | | 0 | |
| improved) | | | | | | | | | |
| | n | 6 | 7 | 4 | | 5 | | 5 | |
| | Mean | 0.3 | 0.3 | 0.3 | | 0.6 | | 0.6 | |
| | Std | 0.52 | 0.76 | 0.50 | | 0.89 | | 0.55 | |
| | Median | 0.0 | 0.0 | 0.0 | | 0.0 | | 1.0 | |
| | Min | 0 | −1 | 0 | | 0 | | 0 | |
| | Max | 1 | 1 | 1 | | 2 | | 1 | |
| Right Thigh | n | 6 | 7 | 5 | | 6 | | 5 | |
| Posterior | | | | | | | | | |
| Day 22 | | | | | | | | | |
| Likert Scale | | | | | | | | | |
| Rating of | | | | | | | | | |
| improvement | | | | | | | | | |
| −1 (Worse) | n (%) | 0 | 2 (28.6) | 0 | | 0 | | 0 | |
| 0 (No change) | n (%) | 2 (33.3) | 5 (71.4) | 4 | (80.0) | 5 | (83.3) | 3 | (60.0) |
| 1 (Improved) | n (%) | 3 (50.0) | 0 | 1 | (20.0) | 1 | (16.7) | 2 | (40.0) |
| 2 (Much | n (%) | 0 | 0 | 0 | | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.6 | −0.3 | 0.2 | | 0.2 | | 0.4 | |
| | Std | 0.55 | 0.49 | 0.45 | | 0.41 | | 0.55 | |
| | Median | 1.0 | 0.0 | 0.0 | | 0.0 | | 0.0 | |
| | Min | 0 | −1 | 0 | | 0 | | 0 | |
| | Max | 1 | 0 | 1 | | 1 | | 1 | |
| Right Thigh | n | 6 | 7 | 5 | | 6 | | 5 | |
| Posterior | | | | | | | | | |
| Day 43 | | | | | | | | | |
| Likert Scale | | | | | | | | | |
| Rating of | | | | | | | | | |
| improvement | | | | | | | | | |
| −1 (Worse) | n (%) | 0 | 0 | 0 | | 1 | (16.7) | 0 | |
| 0 (No change) | n (%) | 2 (33.3) | 5 (71.4) | 3 | (60.0) | 4 | (66.7) | 2 | (40.0) |
| 1 (Improved) | n (%) | 3 (50.0) | 1 (14.3) | 1 | (20.0) | 1 | (16.7) | 3 | (60.0) |
| 2 (Much | n (%) | 0 | 1 (14.3) | 1 | (20.0) | 0 | | 0 | |
| improved) | | | | | | | | | |
| 3 (Very much | n (%) | 0 | 0 | 0 | | 0 | | 0 | |
| improved) | | | | | | | | | |
| | n | 5 | 7 | 5 | | 6 | | 5 | |
| | Mean | 0.6 | 0.4 | 0.6 | | 0.0 | | 0.6 | |
| | Std | 0.55 | 0.79 | 0.89 | | 0.63 | | 0.55 | |
| | Median | 1.0 | 0.0 | 0.0 | | 0.0 | | 1.0 | |
| | Min | 0 | 0 | 0 | | −1 | | 0 | |
| | Max | 1 | 2 | 2 | | 1 | | 1 | |
| Right Thigh | n | 6 | 7 | 5 | | 6 | | 5 | |
| Posterior | | | | | | | | | |
| Day 71 (EOS) | | | | | | | | | |
| Likert Scale | | | | | | | | | |
| Rating of | | | | | | | | | |
| improvement | | | | | | | | | |
| −1 (Worse) | n (%) | 0 | 2 (28.6) | 1 | (20.0) | 1 | (16.7) | 1 | (20.0) |
| 0 (No change) | n (%) | 3 (50.0) | 2 (28.6) | 2 | (40.0) | 3 | (50.0) | 0 | |
| 1 (Improved) | n (%) | 3 (50.0) | 3 (42.9) | 1 | (20.0) | 1 | (16.7) | 4 | (80.0) |

| | | | | | | |
|---|---|---|---|---|---|---|
| 2 (Much | n (%) | 0 | 0 | 0 | 0 | 0 |
| improved) | | | | | | |
| 3 (Very much | n (%) | 0 | 0 | 0 | 0 | 0 |
| improved) | | | | | | |
| | n | 6 | 7 | 4 | 5 | 5 |
| | Mean | 0.5 | 0.1 | 0.0 | 0.0 | 0.6 |
| | Std | 0.55 | 0.90 | 0.82 | 0.71 | 0.89 |
| | Median | 0.5 | 0.0 | 0.0 | 0.0 | 1.0 |
| | Min | 0 | −1 | −1 | −1 | −1 |
| | Max | 1 | 1 | 1 | 1 | 1 |

Hexcel CSS (B) Depth of Depressions.

The Hexel CSS is a photonumeric scale that evaluates 5key morphologic features of cellulite: (A) number of evident depressions, (B) depth of depressions, (C) morphological appearance of skin surface alterations, (D) laxity, flaccidity or sagging of skin, and (E) current classification scale based on medical literature (Hexsel, 2010; Nürnberger, 1978). For this study, only “B—depth of depressions” was assessed. The investigator or qualified designee used the Hexsel CSS to assess the depth of depression of EFP in each buttock or each thigh. The assessment was conducted while the subject was in the standing position with relaxed gluteus muscles. Dimple depth depression was assessed by investigator at the screening visit, Day 22, 43, and 71 using the Hexsel CSS (B) depth of depressions scale and graded as: no depressions (0), superficial depressions (1), medium depth depressions (2) and deep depressions (3). The change from baseline at each post-baseline visit was analyzed and summarized for Hexsel CSS (B) depth of depressions scale score for each treatment area.

The change from baseline in the Hexsel CSS (B) depression depth scale at Days 22, 43, and 71 by injection type was assessed and graded as: no depressions (0), superficial depressions (1), medium depth depressions (2) and deep depressions (3). A negative change in Hexsel CSS (B) from Day 1 to Day 71 indicates an improvement in depression depth. Generally, the left and right buttocks had similar LSMean improvements from baseline in Hexsel CSS (B) dimple depression depth for each injection type. Averaging the left and right buttocks, at Day 71, LSMean depression depth improved in all injection types. Injection type I had the greatest improvement (−1.17), followed by injection type IV (−0.83), and the improvements were statistically significant. These results are summarized in Table 24 below.

TABLE 24
Analysis of Change from Baseline in Dimple Depth Depression (Hexsel CSS (B)) by Treatment Region

| | | Treatment Arm | | | | |
|---|---|---|---|---|---|---|
| Visit | | I | II | III | IV | V |
| Treatment Region | Statistic | (N = 12) | (N = 13) | (N = 11) | (N = 12) | (N = 12) |
| Day 22 | | | | | | |
| Average of Left and Right | | | | | | |
| Buttock | n | 6 | 6 | 6 | 6 | 7 |
| | LS Mean | −0.83 | −0.17 | −0.17 | −0.83 | −0.14 |
| | 95% CI | (−1.28, −0.39) | (−0.61, 0.28) | (−0.61, 0.28) | (−1.28, −0.39) | (−0.56, 0.27) |
| Thigh | n | 5 | 7 | 5 | 6 | 5 |
| | LS Mean | −0.30 | −0.43 | −0.20 | −0.67 | −0.20 |
| | 95% CI | (−0.84, 0.24) | (−0.89, 0.03) | (−0.74, 0.34) | (−1.16, −0.17) | (−0.74, 0.34) |
| Day 43 | | | | | | |
| Average of Left and Right | | | | | | |
| Buttock | n | 6 | 5 | 6 | 6 | 7 |
| | LS Mean | −0.92 | −0.40 | −0.50 | −0.83 | −0.36 |
| | 95% CI | (−1.36, −0.47) | (−0.89, 0.09) | (−0.95, −0.05) | (−1.28, −0.39) | (−0.77, 0.06) |
| Thigh | n | 5 | 7 | 5 | 6 | 5 |
| | LS Mean | −0.40 | −0.43 | −0.40 | −0.67 | −0.60 |
| | 95% CI | (−0.94, 0.14) | (−0.89, 0.03) | (−0.94, 0.14) | (−1.16, −0.17) | (−1.14, −0.06) |
| Day 71 (EOS) | | | | | | |
| Average of Left and Right | | | | | | |
| Buttock | n | 6 | 6 | 6 | 6 | 7 |
| | LS Mean | −1.17 | −0.08 | −0.67 | −0.83 | −0.36 |
| | 95% CI | (−1.61, −0.72) | (−0.53, 0.36) | (−1.11, −0.22) | (−1.28, −0.39) | (−0.77, 0.06) |
| Thigh | n | 6 | 7 | 4 | 5 | 5 |
| | LS Mean | −0.33 | −0.29 | −0.75 | −1.40 | −0.80 |
| | 95% CI | (−0.83, 0.16) | (−0.74, 0.17) | (−1.36, −0.14) | (−1.94, −0.86) | (−1.34, −0.26) |

Note:
EOS = End of Study.
n = Number of subjects having Hexsel CSS score in both baseline and post-baseline visits. Dimple depth depression for each treatment area is assessed by the Investigator using a Hexsel Cellulite Severity Scale (B).
Change from Baseline in dimple depth depression of Hexsel Cellulite Severity Scale (B) is analyzed using linear mixed model with treatment arm, study visits, interaction of treatment arm and study visit as fixed effect. This model will be fitted using the response for the average of left and right of treatment regions (i.e. average of left buttock and right buttock; average of left thigh and right thigh).
Treatment Arm I: Shallow Injection, 3 aliquots; Treatment Arm II: Shallow injection, 1 aliquot; Treatment Arm III: Deep injection, 1 aliquot; Treatment Arm IV: Deep and shallow injections, 5 aliquots; Treatment Arm V: Shallow injection, 4 aliquots.

In injection type I, the buttocks group (average left and right) had an LSMean improvement in dimple depression depth that improved from Day 22 to Day 71. In injection type IV, the buttocks group (average left and right) had an LSMean improvement in dimple depression depth that was maintained from Day 22 to Day 71.

Generally, the left and right thighs had similar LSMean improvements from baseline in Hexsel CSS (B) dimple depression depth for each injection type. Averaging the left and right thighs, at Day 71, LSMean depression depth improved in all injection types. Injection type IV had the greatest improvement (−1.40), followed by injection type V (−0.80), and the improvements were statistically significant. In both injection types IV and V, the thigh group (average left and right) had an LSMean improvement in dimple depression depth that improved from Day 22 to Day 71.

Image Analysis by 3-D Photography.

Assessment of treatment effect was performed by 3-D photography. The investigator or qualified designee photographed each treatment area (both buttocks or both thighs) prior to injections using a standardized digital camera in a standardized manner, before and after marking dimples and injection sites on treatment Day 1, 22, and 43. A single set of photographs were taken of each treatment area at screening and all other non-dosing visits. The photographs taken at Day 4, 8, 15, 22, 43, and 71 (end of study/ET) were reviewed by a central assessor blinded to the treatment arm and study visit day.

The standard Image Analysis (IA) procedures used to evaluate bruising, volume, surface area, and max length/width are discussed below. The camera system used for this study was an IntelliStudio available from Canfield Scientific equipped with custom lighting, a Vectra M1 camera, and Canfield Capture software (v. 3.5). The image types, attributes, views, and visit windows were defined and inputted directly into a Digital Monitoring System (DMS) within the study protocol. The study set-up in DMS contained the following:

| Attribute Type | |
|---|---|
| (category) | Attribute Names |
| Image Type | 2-D, 3-D, Contact Sheets |
| View | ID Card, Left Buttock Posterior, Right |
| | Buttock Posterior, Left Thigh Lateral, Left |
| | Thigh Oblique, Left Thigh Posterior, Right |
| | Thigh Posterior, Right Thigh Oblique, Right |
| | Thigh Lateral |
| Real Time Monitoring | Real Time Monitoring |

Images were captured and staged into DMS and available for review. Photographic visits included: Screening, Day 1/Pre-Marking, Day 1/Post-Marking, Day 4, Day 8, Day 15, Day 22/Pre-Marking, Day 22/Post Marking, Day 43/Pre-Marking, Day 43 Post-Marking, and Day 71/ET.

Dimple Analysis:

Images were reviewed to ensure a primary dimple was marked with an “X” as instructed in each quadrant. If a primary dimple was not marked, the following steps were taken:
1. The image(s) and place the images were exported in a PowerPoint.
2. The site was contacted and asked the site's investigator to mark the primary dimple with an X on the image within the PowerPoint.
3. The correspondence was saved and share with the image analysis (IA) team.
   Dimple analysis was performed on Days 1 (pre-marking), 22, 43 and 71.

An IAT used the Day 1-Post marking image as a reference to determine the location of the target dimple on the Day 1-Pre marking image. A tracing was made around the border of concavity of the dimple on the Day 1-Pre marking. The dimple tracing was transposed on to the Day 22-Pre Marking, Day 43-Pre marking, and Day 71/ET images. This tracing was used to measure the (i) maximum length (i.e., the longest straight line distance across the dimple); (ii) maximum width (i.e., the longest straight line distance perpendicular to the maximum length measurement); (iii) surface area of the dimple; and (iv) volume between the base of the dimple and the interpolated surface.

Bruising Analysis:

Images were reviewed to ensure a white reference label was present in each image and outside of the bruise area. If the reference label was not present or obstructing the bruised area, a reshoot was requested if applicable for that visit. Bruise analysis was performed on Days 1 (pre-marking), 4, 8, and 15.

Site Marking Analysis:

Images were reviewed to ensure dimples were marked at the Day 1 Post-Marking visit. If dimples were not marked, a reshoot was requested if applicable for that visit. Site Marking Analysis:

Image Analysis (IA) Workflow:

All images selected for analysis were assigned random tracking numbers which blinded the image analysis technicians (IAT) to information, such as investigative site, subject secondary identifier, and/or treatment arm. The analysis procedure performed on a particular visit was pre-determined based on visit name. Dimple analysis was performed on the pre-identified, primary dimple and will include measurements of volume, surface area, and max length/width. Bruise analysis included L*a*b* color measurements in the perceived bruised area and an area outside the perceived bruise on unaffected skin. Site Marking Analysis consisted of max length/width in addition to a surface area measurement of the tracing.

Image Registration:

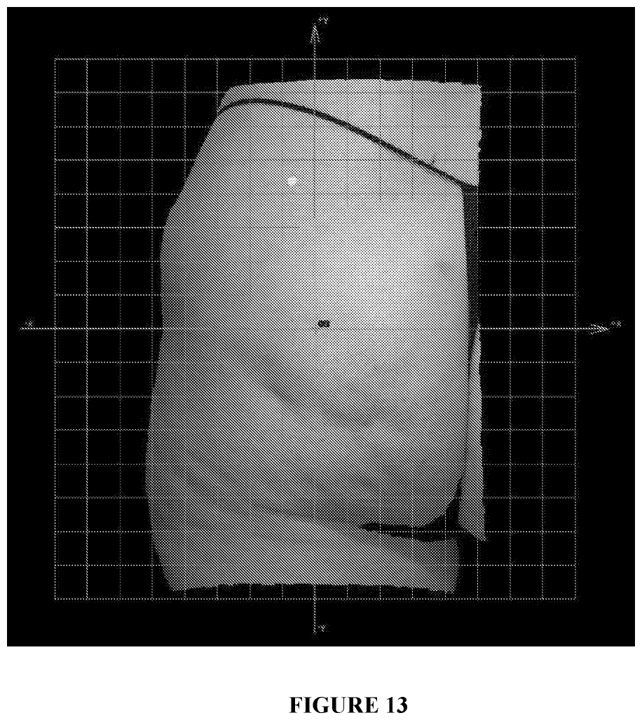

Upon receiving a Day 1 Pre-Marking (Baseline) visit, a IAT opened the pre-marking image in Vectra Analysis Module (VAM) software and centered the image to grid in 3-D space. Using the grid as a reference, the IAT positioned the Baseline image so that the approximate center of the image was placed at the grid's origin. The thigh/buttock faced forward in the +z-direction, the upper thigh/buttock pointed in the +y-direction and the lower thigh/buttock pointed in the −y-direction (FIG. 13). All subsequent time points, including the Day 1 post-marking image were registered to Baseline image's position in the grid using an algorithmic registration function.

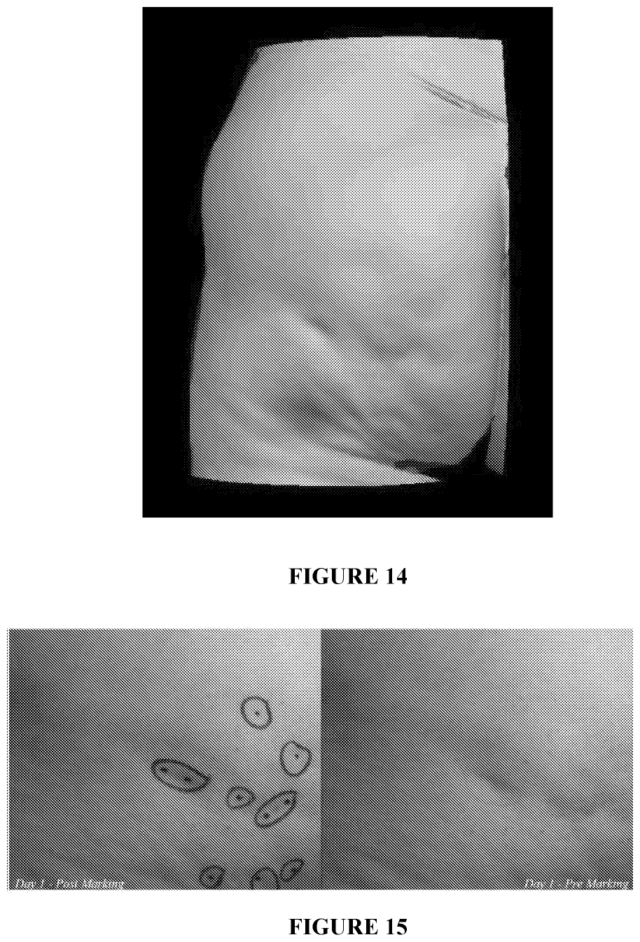

A color-by-distance map was produced within VAM which the IAT used to review registration. The color-by-distance map represented the distance between the two (2) image models based on a color scale of +5 to −5 millimeter (mm) (FIG. 14). The IAT ensured the majority of the image is colored green, indicating there was a negligible distance between the two images and that were properly aligned.

| Color | Distance from Baseline |
|---|---|
| Green | 0 or negligible distance |
| Cyan → Blue | Positive Distance (Up to +5 mm) |
| Yellow → Red | Negative Distance (Down to −5 mm) |

Surface Tracking Processing:

Following image registration, the Day 1 (Pre-Marking), Day 4, Day 8, Day 15, Day 22, Day 43 and Day 71 images received surface tracking processing. The IAT first used a script to create a high-density mesh template of the Day 1, Baseline image. One Follow-Up image at a time was opened in the VAM software along with the Baseline and mesh template. Tracking seed landmarks were placed on identifiable skin features as references. The tracking script was run to programmatically create and save three (3) new 3-D images, a tracked Baseline image, a tracked Follow-Up image, and a Quality Control (QC) image which was the shape of the tracked Follow-Up image and conveys information regarding tracking quality.

A quality check IAT reviewed the quality of the tracked Follow-Up image and QC image to verify a successful tracking. For visits that received dimple analysis, the IAT also overlaid the QC image over the tracked Follow-Up image to ensure there were no large holes on the QC image in the region of the primary dimple. A hole in the mesh of the QC image indicated a loss of tracking information in the area of drop out. In the event that the IAT deemed tracking unacceptable due to a loss of information within an area for analysis, the tracking seed landmarks were adjusted and the tracking script run again. The IAT noted unavoidable instances of poor surface tracking quality which stemmed from glare, deep shadow, imprints in skin left by clothing, large changes in subject position or large change in skin pigmentation. Holes in the mesh of the QC image were expected in areas of strong bruising.

Primary Dimple Area of Interest (AOI) Delineation:

Following successful surface tracking, a IAT opened the tracked Baseline image and registered Day 1-Post Marking image in VAM. On the Day 1-Post Marking image, the IAT traced the outer border of the primary dimple site marking. This tracing was used to measure surface area of the site marking, the largest axis across the site marking (Max Dimple Length) and the length of its largest perpendicular axis (Max Dimple Width). This tracing was not used to delineate an AOI for the Dimple or Bruise analysis. In cases where a primary dimple was not identified by the site, the IAT traced the dimple he/she perceived to be largest.

The IAT used the Day 1-Post Marking image as a reference to locate the target dimple on the Day 1-Pre-Marking image. The IAT then traced the boundary of the primary dimple on the tracked, pre-marking image. The tracing was adjusted as necessary until it was just outside the ridge at which the dimple became concave and delineated the primary dimple AOI for dimple analysis. (FIG. 15). The dimple tracing on the tracked, pre-marking image was transposed onto the Day 22, Day 43 and Day 71 Follow-Up images based on each Follow-Up image's unique surface tracking relationship to the Baseline (FIG. 16). The IAT noted any instances of poor image quality affecting the transposed AOI.

Bruise Area of Interest (AOI) Delineation:

The IAT created a “Bruised Tissue” AOI, a single continuous tracing around the perimeter of the largest perceptible bruise on the Day 4 image (FIG. 17). The IAT traced the perimeter where he/she could determine a difference in skin pigmentation between bruised skin and unaffected skin. Skin was considered bruised if it exhibited blue/purple, green, or yellow/brown pigmentation distinguishable from the surrounding skin tone. In the case where no bruise could be distinguished on the Day 4 image, the subject was failed for the purposes of the bruise analysis.

Additionally, the IAT created a second tracing, the “Normal Tissue” AOL. The Normal Tissue AOI was created on an area of skin unaffected by skin texture distortions caused by clothing compression lines, abnormal redness, or skin features such as acne or scar tissue. Where possible and applicable, the Normal Tissue AOI attempted to match any shadowing, shine, or glare present across the curvature of the Day 1 Pre-Marking Bruised Tissue AOL. The size and shape of the Normal Tissue AOI varied across subjects depending on the amount of natural appearing skin available. Once traced, the IAT transposed both the Bruised Tissue and Normal Tissue AOI from the Day 4 image to the Day 1-Pre-Marking, Day 8 and Day 15 images based on their surface tracking relation to the Day 4 image. In the event that a Bruised Tissue AOI or Normal Tissue AOI extended beyond the edges of other bruise analysis visits for a particular subject or side of subject, the AOIs were adjusted until consistent across all visits.

The IAT manually traced the white reference label on the Day 1-Pre Marking, Day 4, Day 8 and Day 15 images and labeled the tracing “White.” If the white reference label was placed within the bruised area, the IAT traced the border of the label and subtracted it from the bruise AOI at each affected visit. In the case where no white reference label was present on an image, that image was failed for purposes of the bruise analysis.

Quality Control (QC) Checks:

The QC checks were performed by a IAT independent of the IAT who performed analysis on the images. A separate set of QC checks was performed depending on the analysis procedure for a given visit. Any adjustments made by the QC IAT throughout the QC checks were noted and re-QC'd by another IAT before passing to analysis. Prior to beginning QC, the QC IAT reviewed any notes made by the original IAT regarding image quality issues.
The Day 1—Pre-Marking image was reviewed by the QC IAT for proper registration to the VAM grid. The QC IAT also referenced the Day 1 (Post-Marked) image to ensure the primary dimple has been properly identified and traced on the Day 1 (Pre-Marked) image. The QC IAT reviewed the primary dimple AOI to confirm the perimeter of the AOI fell just outside the ridge at which the dimple becomes concave. The QC IAT then checked for the presence of both a Normal Tissue and Bruised Tissue AOI, ensuring the Normal Tissue AOI was a representative color sample. The QC IAT adjusted the perimeter of each AOI only when necessary, noting any changes that were made. If changes to the AOI or landmark placement were made, the IAT transposed the updated AOI on the appropriate Follow-Up images.
The Day 1—Post Marking image were reviewed by the QC IAT to ensure a proper tracing was made along the outer border of the site dimple marking, adjusting the AOI if an improper selection was made. Improper selections included a tracing on the inner border of a site marking or the tracing of dimple not specified as the primary dimple.
The Day 22, Day 43 and Day 71 images were reviewed by the QC IAT for acceptable tracking quality. The QC IAT referred to the QC Follow-Up image to ensure there were no holes on the QC image within the primary dimple AOL. If a hole fell within the primary dimple AOI, the QC IAT added a “poor tracking quality” note for the visit. In cases where the majority of the primary dimple AOI consisted of surface tracking dropout, the QC IAT recommended the image for failure.
The Day 4, Day 8 and Day 15 Follow-Up images were reviewed for proper bruise delineation first. The QC IAT adjusted the perimeter of the bruise AOI on the Day 4 image in cases where the original perimeter consistently encompassed more of the bruise discoloration or when the perimeter encompassed unbruised skin. The QC IAT noted any adjustments to the Bruised Tissue AOI and also recommended a subject for bruise analysis failure cases where there was no identifiable border between the bruised and normal skin color. The QC IAT then checked for the presence of a Normal Tissue AOI, ensuring proper naming of the AOI and that the Normal Tissue AOI is a consistent and representative color sample. Any abnormal, unavoidable coloration within the base skin AOI was noted by the QC IAT. If any adjustments were made to either the Bruised Tissue or Normal Tissue AOI on the Day 4 image, the IAT transposed the adjusted tracings to the Day 1—Pre Marking, Day 8 and Day 15 images. Finally the IAT ensured the white reference label on each image was traced and properly named “White.” In cases where the reference label was placed within the bruised tissue, the QC IAT ensured the label was subtracted from the Bruised Tissue AOL. Images without a white reference label were failed for bruise analysis.

Analysis Measurements:

After images passed QC, the analysis IAT reviewed notes from the QC IAT. The analysis IAT then ran a scripted analysis procedure. The procedure and analysis end points differed between images depending on the given visit. For dimple volume analysis, an interpolated surface was created across the top of the primary dimple AOL. A volume measurement represented the space between the interpolated surface and base of the dimple.

Surface area was measured as the total surface area of the primary dimple AOI or site tracing AOI in the case of the Day 1-Post Marking image. The Max Dimple Length was measured as the largest point to point axis across the primary dimple AOL. The Max Dimple Width was measured as the largest point-to-point distance perpendicular to the Max Dimple Length Axis.

Bruise analysis consisted of two L*a*b* color measurements. L*a*b* color values were measured within the Bruised Tissue and Normal Tissue AOIs (FIG. 18(A)). The following table summarizes the analyses performed (Table 25).

TABLE 25
Summary of the Bruise Analysis

| Analysis Category | Analysis Name | Analysis Units | Analysis Time Point |
|---|---|---|---|
| Dimple | Volume | cc | Day 1 Pre-Marking, Day 22, |
| | | | Day 43 and Day 71 |
| Dimple | Surface Area | mm² | Day 1 Pre-Marking, Day 1-Post |
| | | | Marking, Day 22, Day 43 and |
| | | | Day 71 |
| Dimple | Max Dimple Length | mm | Day 1 Pre-Marking, Day 1 |
| | (Max Length Straight Line) | | Post-Marking, Day 22, Day |
| | | | 43 and Day 71 |
| Dimple | Max Dimple Width | mm | Day 1 Pre-Marking, Day 1 |
| | (Max Perpendicular Width Straight | | Post-Marking, Day 22, Day |
| | Line) | | 43 and Day 71 |
| Color | Bruised Tissue L* | L* color value | Day 1-Pre Marking, Day 4, |
| | (BruisedL) | | Day 8, Day 15 |
| Color | Bruised Tissue a* | a* color value | Day 1-Pre Marking, Day 4, |
| | (Bruiseda) | | Day 8, Day 15 |
| Color | Bruise Tissue b* | b* color value | Day 1-Pre Marking, Day 4, |
| | (Bruisedb) | | Day 8, Day 15 |
| Color | Normal Tissue L* | L* color value | Day 1-Pre Marking, Day 4, |
| | (Normal L) | | Day 8, Day 15 |
| Color | Normal Tissue a* | a* color value | Day 1-Pre Marking, Day 4, |
| | (Normala) | | Day 8, Day 15 |
| Color | Normal Tissue b* | b* color value | Day 1-Pre Marking, Day 4, |
| | (Normalb) | | Day 8, Day 15 |

Accepting Data:

Once image analysis was complete, the analyzing IAT either accepted the image or failed the image for analysis. Images could be partially failed, e.g., a Day 1 Pre-Marking visit may have failed bruising analysis for no visible bruising at Day 4, but have still been acceptable for dimple analysis. All failures received a mandatory Failure Note detailing why the image model was unacceptable for analysis. Image failure may be due to factors such as poor image quality, poor tracking quality or obstructions within an AOI. Images were recommended for failure by the QC IAT and reviewed by the analyzing IAT. If the analysis IAT agreed with the failure recommendation, the image was failed.

Bruising Analysis.

Bruised tissue and normal tissue were assessed on 3-D photographs using two L*A*B* color measurements at Day 1, 4,8, and 15 (FIG. 18(B)). The greater the L*A*B* color intensity measurement, the worse the bruising. The change in visual perception between two colors of the bruised tissue versus the normal tissue for each treatment area and injection type are presented in Table 26 below.

TABLE 26
Summary of Change in Color Coordinates (Bruised Tissue
vs. Normal Tissue) - Bruise Analysis-Safety Population

ΔE (Bruised Tissue

| vs Normal Tissue) | | Injection Type | | | | |
|---|---|---|---|---|---|---|
| Injection Type | | I | II | III | IV | V |
| Visit | Statistic | (N = 12) | (N = 13) | (N = 12) | (N = 13) | (N = 13) |
| Left Buttock Posterior | | | | | | |
| Day 1 Pre-marking | n | 5 | 6 | 6 | 6 | 7 |
| | Mean | 4.585 | 3.916 | 3.900 | 3.834 | 4.135 |
| | Std | 1.9614 | 1.2366 | 1.1882 | 1.3168 | 1.1533 |
| | Median | 5.277 | 3.937 | 4.059 | 3.692 | 4.323 |
| | Min | 1.47 | 2.35 | 2.36 | 2.49 | 2.39 |
| | Max | 6.46 | 5.29 | 5.31 | 5.65 | 5.86 |
| Day 4 | n | 5 | 6 | 6 | 6 | 7 |
| | Mean | 19.582 | 8.590 | 23.013 | 22.255 | 20.345 |
| | Std | 8.7930 | 6.2551 | 7.5632 | 11.5855 | 6.6155 |
| | Median | 22.083 | 6.813 | 22.022 | 26.319 | 22.082 |
| | Min | 7.69 | 4.10 | 15.84 | 4.55 | 10.59 |
| | Max | 28.21 | 21.04 | 35.32 | 32.64 | 29.07 |
| Day 8 | n | 5 | 6 | 6 | 6 | 7 |
| | Mean | 12.893 | 10.032 | 15.871 | 16.560 | 14.702 |
| | Std | 6.2732 | 9.0029 | 9.8693 | 8.5537 | 7.8751 |
| | Median | 10.415 | 6.179 | 12.666 | 16.839 | 14.454 |
| | Min | 6.09 | 1.92 | 8.85 | 2.02 | 4.15 |
| | Max | 20.11 | 24.84 | 35.63 | 24.97 | 28.67 |
| Day 15 | n | 5 | 6 | 6 | 6 | 6 |
| | Mean | 9.092 | 5.005 | 7.481 | 8.303 | 7.612 |
| | Std | 4.7708 | 2.5981 | 3.8655 | 5.6299 | 4.5750 |
| | Median | 9.443 | 5.032 | 8.089 | 8.129 | 6.121 |
| | Min | 1.61 | 1.37 | 1.65 | 0.88 | 4.16 |
| | Max | 14.84 | 8.80 | 13.29 | 16.86 | 16.59 |
| Right Buttock Posterior | | | | | | |
| Day 1 Pre-marking | n | 5 | 6 | 6 | 6 | 7 |
| | Mean | 3.381 | 3.709 | 4.805 | 3.123 | 4.411 |
| | Std | 1.6830 | 1.1922 | 0.9015 | 1.1024 | 1.9656 |
| | Median | 3.064 | 3.787 | 4.760 | 2.891 | 4.549 |
| | Min | 1.68 | 1.99 | 3.80 | 1.71 | 1.53 |
| | Max | 6.20 | 5.21 | 5.98 | 4.95 | 6.63 |
| Day 4 | n | 5 | 6 | 6 | 6 | 7 |
| | Mean | 18.676 | 10.759 | 25.424 | 20.422 | 18.392 |
| | Std | 7.0131 | 9.0977 | 9.0672 | 8.9211 | 9.0280 |
| | Median | 15.081 | 7.538 | 22.538 | 20.743 | 18.761 |
| | Min | 11.24 | 1.11 | 18.46 | 9.31 | 2.51 |
| | Max | 27.32 | 25.11 | 42.05 | 31.68 | 28.95 |
| Day 8 | n | 5 | 6 | 6 | 6 | 7 |
| | Mean | 11.350 | 12.915 | 15.387 | 14.160 | 13.821 |
| | Std | 6.2875 | 12.8715 | 6.5965 | 8.0371 | 6.7135 |
| | Median | 7.859 | 5.618 | 13.135 | 12.424 | 14.092 |
| | Min | 5.57 | 3.90 | 10.24 | 5.04 | 4.21 |
| | Max | 18.88 | 33.04 | 27.73 | 28.41 | 23.74 |
| Day 15 | n | 5 | 6 | 6 | 6 | 6 |
| | Mean | 6.059 | 7.140 | 8.592 | 9.040 | 9.413 |
| | Std | 3.5688 | 4.8814 | 4.7793 | 2.5947 | 3.8992 |
| | Median | 5.583 | 5.591 | 7.116 | 9.635 | 9.246 |
| | Min | 1.37 | 3.58 | 2.22 | 4.07 | 5.40 |
| | Max | 10.64 | 16.70 | 14.43 | 11.31 | 14.10 |
| Left Thigh | | | | | | |
| Day 1 Pre-marking | n | 5 | 7 | 5 | 7 | 6 |
| | Mean | 4.396 | 3.211 | 4.266 | 3.864 | 3.629 |
| | Std | 0.5627 | 1.1676 | 1.5690 | 0.5736 | 1.6866 |
| | Median | 4.307 | 3.180 | 3.894 | 3.875 | 4.073 |
| | Min | 3.82 | 1.40 | 2.34 | 3.07 | 0.33 |
| | Max | 5.20 | 4.39 | 6.27 | 4.60 | 5.21 |
| Day 4 | n | 4 | 7 | 5 | 7 | 6 |
| | Mean | 24.505 | 15.633 | 13.530 | 14.625 | 10.410 |
| | Std | 5.9563 | 3.5185 | 6.0469 | 7.5387 | 8.9790 |
| | Median | 26.031 | 16.621 | 12.845 | 15.374 | 7.361 |
| | Min | 16.19 | 10.14 | 6.21 | 6.17 | 1.19 |
| | Max | 29.77 | 20.41 | 21.41 | 24.54 | 23.46 |
| Day 8 | n | 5 | 7 | 4 | 6 | 5 |
| | Mean | 13.431 | 11.262 | 11.276 | 10.301 | 9.518 |
| | Std | 6.0507 | 2.5014 | 6.7819 | 4.9684 | 6.5803 |
| | Median | 13.740 | 10.462 | 13.590 | 9.122 | 7.330 |
| | Min | 6.35 | 8.69 | 1.64 | 3.63 | 1.79 |
| | Max | 21.66 | 16.45 | 16.29 | 18.22 | 18.64 |
| Day 15 | n | 5 | 7 | 4 | 7 | 4 |
| | Mean | 4.772 | 5.944 | 5.021 | 6.972 | 5.870 |
| | Std | 3.8296 | 2.1660 | 3.5217 | 2.5803 | 2.0773 |
| | Median | 3.411 | 6.538 | 5.110 | 7.510 | 6.554 |
| | Min | 1.55 | 1.62 | 1.33 | 3.49 | 2.88 |
| | Max | 11.38 | 7.93 | 8.54 | 10.63 | 7.49 |
| Right Thigh | | | | | | |
| Day 1 Pre-marking | n | 5 | 7 | 5 | 7 | 5 |
| | Mean | 3.896 | 3.044 | 3.752 | 2.963 | 3.936 |
| | Std | 1.1756 | 0.8559 | 1.5334 | 0.9839 | 0.9798 |
| | Median | 3.944 | 2.967 | 4.027 | 3.029 | 4.090 |
| | Min | 2.68 | 1.99 | 1.59 | 1.10 | 2.39 |
| | Max | 5.11 | 4.24 | 5.78 | 4.16 | 4.99 |
| Day 4 | n | 4 | 7 | 5 | 7 | 5 |
| | Mean | 22.666 | 14.299 | 18.002 | 14.274 | 12.133 |
| | Std | 5.7515 | 3.3324 | 7.7703 | 4.2206 | 5.5537 |
| | Median | 24.286 | 14.727 | 16.820 | 13.699 | 9.790 |
| | Min | 14.63 | 8.19 | 8.68 | 10.03 | 6.01 |
| | Max | 27.46 | 19.41 | 28.83 | 22.55 | 19.87 |
| Day 8 | n | 5 | 7 | 5 | 7 | 5 |
| | Mean | 10.885 | 10.883 | 14.257 | 9.847 | 7.567 |
| | Std | 3.5219 | 3.5317 | 8.3541 | 3.4280 | 4.5560 |
| | Median | 11.460 | 10.734 | 10.995 | 9.565 | 4.661 |
| | Min | 5.95 | 6.14 | 5.85 | 4.74 | 3.95 |
| | Max | 15.30 | 15.21 | 24.10 | 15.43 | 12.77 |
| Day 15 | n | 5 | 7 | 4 | 7 | 4 |
| | Mean | 4.890 | 6.613 | 6.987 | 6.234 | 4.829 |
| | Std | 2.0320 | 1.3229 | 4.1124 | 2.3766 | 0.6311 |
| | Median | 4.107 | 6.890 | 6.270 | 6.526 | 4.784 |
| | Min | 2.99 | 4.82 | 2.82 | 2.52 | 4.25 |
| | Max | 7.99 | 8.06 | 12.58 | 8.73 | 5.50 |

Note:
NE = Not estimable due to insufficient data.
Δ E (Color difference between-Bruised vs. Normal) = SQRT [(L*_{B} − L*_{N})² + (A*_{B} − A*_{N})² + (B*_{B} − B*_{N}) ²], where L*_{B} = Bruised Tissue L*, L*_{N} = Normal Tissue L*, A*_{B} = Bruised Tissue A*, A*_{N} = Normal Tissue A*, B*_{B} = Bruised Tissue B*, B*_{N} = Normal Tissue B*
Injection type I: Shallow Injection, 3 aliquots; Injection type II: Shallow injection, 1 aliquot; Injection type III: Deep injection, 1 aliquot; Injection type IV: Deep and shallow injections, 5 aliquots; Injection type V: Shallow injection, 4 aliquots.

At Day 1 pre-marking, the color coordinates were similar across all injection types. In the buttocks (left and right), bruising peaked at Day 4 in injection types I, III, IV, and V; and peaked at Day 8 in injection type II. Buttock bruising was the greatest in injection types III and IV. Buttock bruising was substantially reduced at Day 15 in all injection types. In the thigh (left and right), bruising peaked at Day 4 in all injection types. Thigh bruising was the greatest in injection type I. Thigh bruising was substantially reduced at Day 15 in all injection types.

Dimple Volume Analysis.

For each treated buttock and thigh, four dimple parameters including the maximum length (largest straight line distance across the dimple), maximum width (largest straight line distance perpendicular to the maximum length measurement), surface area, and volume (between the base of the dimple and an interpolated surface) of the target dimple were assessed. In each thigh, the maximum length, maximum width, surface area, and volume of the target dimple in the lateral, oblique, and posterior views were measured. A negative change from Day 1 indicates an improvement. The results are summarized in Table 27 below.

TABLE 27
Analysis of Dimple Volume by Treatment Region

| | | Treatment Arm | | | | |
|---|---|---|---|---|---|---|
| Visit | | I | II | III | IV | V |
| Treatment Region | Statistic | (N = 12) | (N = 13) | (N = 11) | (N = 12) | (N = 12) |
| Day 22 | | | | | | |
| Average of Left and Right | | | | | | |
| Buttock | n | 6 | 6 | 6 | 6 | 7 |
| | LS Mean | −0.01 | <−0.001 | −0.01 | −0.01 | −0.04 |
| | 95% CI | (−0.04, 0.01) | (−0.03, 0.02) | (−0.04, 0.01) | (−0.04, 0.01) | (−0.06, −0.01) |
| Thigh | n | 5 | 7 | 5 | 6 | 5 |
| | LS Mean | −0.02 | −0.04 | 0.00 | −0.01 | −0.04 |
| | 95% CI | (−0.07, 0.02) | (−0.08, <−0.0001) | (−0.05, 0.05) | (−0.06, 0.03) | (−0.09, 0.01) |
| Day 43 | | | | | | |
| Average of Left and Right | | | | | | |
| Buttock | n | 6 | 5 | 6 | 6 | 7 |
| | LS Mean | −0.02 | −0.02 | −0.01 | −0.01 | −0.04 |
| | 95% CI | (−0.05, 0.00) | (−0.05, 0.00) | (−0.04, 0.01) | (−0.04, 0.01) | (−0.07, −0.02) |
| Thigh | n | 5 | 7 | 5 | 6 | 5 |
| | LS Mean | −0.03 | −0.04 | 0.01 | −0.02 | −0.06 |
| | 95% CI | (−0.08, 0.02) | (−0.08, 0.00) | (−0.04, 0.06) | (−0.07, 0.02) | (−0.11, −0.01) |
| Day 71 (EOS) | | | | | | |
| Average of Left and Right | | | | | | |
| Buttock | n | 6 | 6 | 6 | 4 | 7 |
| | LS Mean | −0.02 | −0.04 | −0.01 | −0.02 | −0.05 |
| | 95% CI | (−0.05, 0.00) | (−0.06, −0.01) | (−0.04, 0.02) | (−0.05, 0.01) | (−0.07, −0.02) |
| Thigh | n | 6 | 7 | 4 | 5 | 5 |
| | LS Mean | −0.02 | −0.02 | 0.04 | −0.02 | −0.05 |
| | 95% CI | (−0.07, 0.02) | (−0.07, 0.02) | (−0.02, 0.09) | (−0.07, 0.03) | (−0.09, 0.00) |

Note:
EOS = End of Study.
Volume of Dimple Depth Depression from Day 1 pre-marking is analyzed using linear mixed model with treatment arm, study visit, interaction of treatment arm and study visit as fixed effects. This model will be fitted using the measurement for the average of left and right of treatment regions (i.e. average of left buttock posterior and right buttock posterior; average of left thigh lateral, left thigh oblique, left thigh posterior, right thigh lateral, right thigh oblique, and right thigh posterior).
Treatment Arm I: Shallow Injection, 3 aliquots; Treatment Arm II: Shallow injection, 1 aliquot; Treatment Arm III: Deep injection, 1 aliquot; Treatment Arm IV: Deep and shallow injections, 5 aliquots; Treatment Arm V: Shallow injection, 4 aliquots.

In the buttocks, all injection types produced a mean percent improvement from baseline in dimple surface area, with the greatest improvement occurring in injection type V (left buttock) and injection type IV (right buttock). In the thighs, the greatest improvement in surface area occurred with injection type IV (left thigh) and injection type II (right thigh), while injection type III produced no improvement in surface area in the left thigh.

Generally, the left and right buttocks had similar LSMean improvements from baseline in dimple volume for each injection type. Averaging the left and right buttocks, at Day 71, all injection types showed an improvement in dimple volume compared with baseline, with injection types II and V having a significant improvement. In general, the improvement in dimple volume of the buttocks compared with baseline tended to improve from Day 22 to Day 71, except for injection type III.

Generally, the left and right thighs had similar LSMean improvements from baseline in dimple volume for all injection types. Averaging the left and right thighs, at Day 71, injection types I, II, IV, and V showed an improvement in dimple volume compared with baseline. In the thigh, the dimple volume differences between treatment arms were very small, so comparisons between injection types cannot be made. In general, the improvement in dimple volume of the thighs compared with baseline tended to improve from Day 22 to Day 43, except for injection type III.

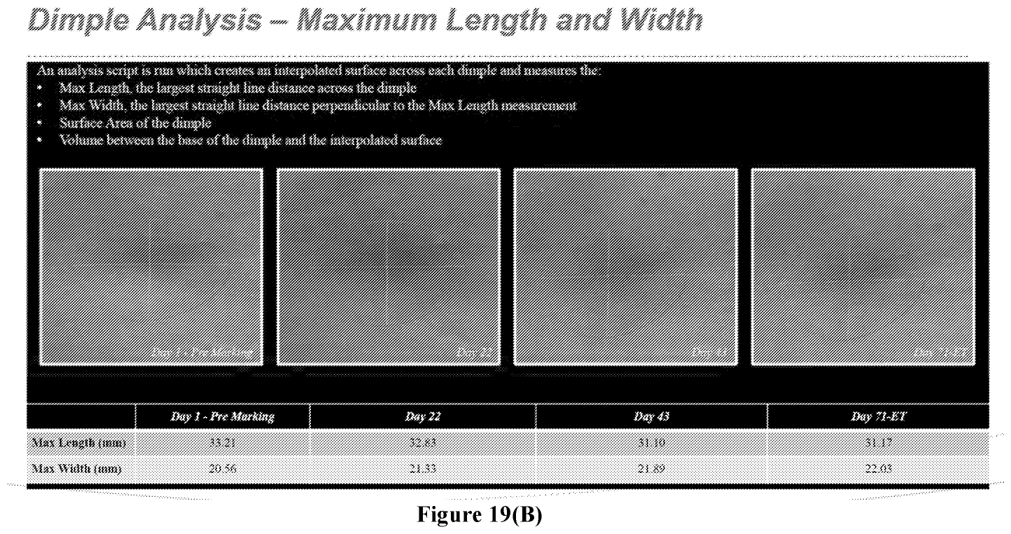

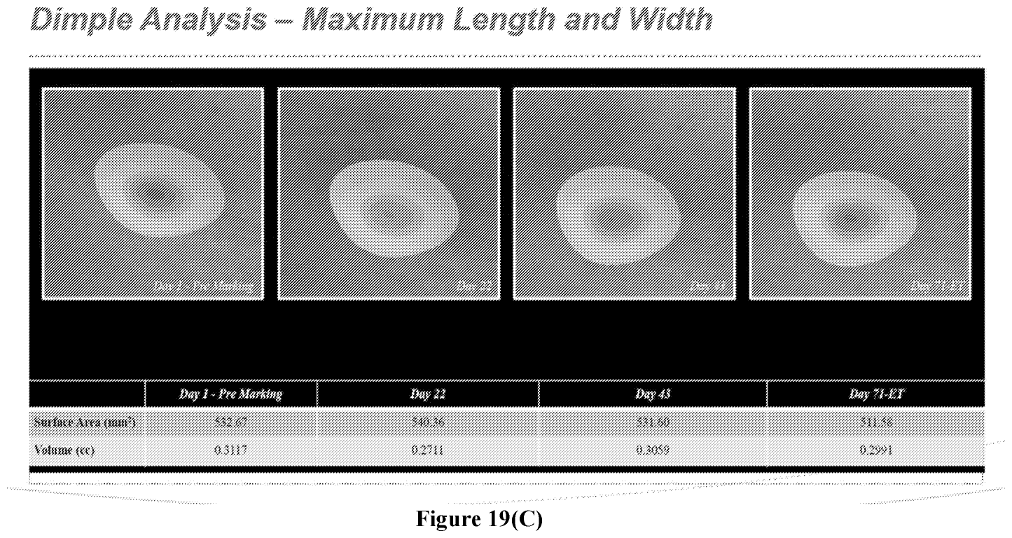

The observed and change from Day 1 pre-marking image in dimple analysis parameters, maximum length, maximum width, surface area, and volume between the dimple base and interpolated surface, are summarized at Day 22, 43, and 71 by treatment area and injection type using descriptive statistics (FIGS. 19(A)-19(C)). In addition, the volume was analyzed using the linear model. A listing of dimple analysis parameters is provided.

Results and Conclusions.

The primary objective of this study was to assess the treatment effects of CCH when administered by different injection techniques in subjects with EFP. This objective was assessed by evaluating the improvement in aesthetic appearance of each treatment using 3-D photography and a Likert score, analyzing the change dimple parameters for each treatment arm using 3-D photography, and evaluating the change in dimple depth depression score with the Hexsel CSS (B) depression depth scale.

Efficacy/Buttock:

Assessing improvement in aesthetic appearance using a 5-point Likert scale and averaging the left and right buttocks, at Day 71 (EOS), all treatments showed an improvement in aesthetic appearance compared with baseline, with injection types I, II, III, and V having a significant improvement.

Analyzing the change in dimple parameters and averaging the left and right buttocks, at Day 71, all treatments showed an improvement in dimple volume compared with baseline, with Treatments II and V having a significant improvement. Also, in the buttocks, all injection types produced a mean percent improvement from baseline in dimple surface area, with injection type 5 producing the greatest improvement.

Evaluating the change in dimple depression depth using the Hexsel CSS (B) depression depth scale, and averaging the left and right buttocks, at Day 71, LSMean depression depth improved in all treatments. However, injection type I had the greatest improvement (−1.17) in the buttock, followed by injection type IV (−0.83), and the improvements were statistically significant. Taken together, in the buttocks, injection types I and V were most efficacious based on each having the greatest benefit on ≥2 efficacy endpoints. Namely, injection type I showed the best efficacy according to improvement in aesthetic appearance (Likert score) and improvement in dimple depression depth (Hexsel CSS (B)), and injection type V showed the best efficacy according to improvement in aesthetic appearance (Likert score) and dimple parameter assessment (surface area and volume).

Efficacy/Thigh:

Assessing improvement in aesthetic appearance using a 5-point Likert scale and averaging the left and right thighs, at Day 71 (EOS), all injection types showed an improvement in aesthetic appearance compared with baseline, with injection type I having a significant improvement. However, it was hypothesized that the Likert scale method of using three pairs to generate one entire thigh score is flawed, so the Likert scores for thigh were reassessed by a blinded central assessor to produce one Likert score per image.

Analyzing the change in dimple parameters and averaging the left and right thighs, at Day 71, injection types I, II, IV, and V showed an improvement in dimple volume compared with baseline. However, the dimple volume differences between treatment arms are small, so comparisons between injection types cannot be made.

Evaluating the change in dimple depression depth using the Hexsel CSS (B) and averaging the left and right thighs, at Day 71, LSMean depression depth improved in all injection types. However, injection type IV had the greatest improvement in the thigh (−1.40), followed by injection type V (−0.80), and the improvements were statistically significant. Taken together, in the thighs, injection types I and V were most efficacious based on each having the greatest benefit on ≥2 efficacy endpoints. Namely, injection type I showed the best efficacy according to improvement in aesthetic appearance (Likert score) and improvement in dimple depression depth (Hexsel CSS [B]), and injection type V showed the best efficacy according to improvement in aesthetic appearance (Likert score) and dimple parameter assessment (surface area and volume).

Safety:

The secondary objective of this study was to assess the safety of CCH when administered using different injection techniques in subjects with EFP. This objective was accomplished by adverse events, injection site reactions/local tolerability, vital signs, ECG, clinical laboratory parameters (including hematology, blood chemistry, and urinalysis), bruising, and immunogenicity (binding antibodies and neutralizing antibodies). For an injection type to be considered safe, it needs to be safe and well tolerated in the overall population irrespective of region.

An evaluation of the adverse events shows that injection site nodule was most frequently reported in injection types II and III (10 [76.9%] subjects and 39 occurrences, and 10 [83.3%] subjects and 26 occurrences; respectively). Injection site discoloration was reported most frequently in injection type II (6 subjects [46.2%], 13 occurrences). Injection type III had the greatest proportion of subjects with severe SOC General Disorders and Administration Site Conditions. Specifically, injection type III had the greatest proportion of subjects with severe injection site bruising (6 subjects, 50%), injection site pain (4 subjects, 33.3%), and injection site nodules (3 subjects, 25.0%). Injection type II had the greatest frequency of treatment-related TEAEs that had a duration of >21 days (31 events, 16.7%) and the greatest median duration of all treatment-related TEAEs (12.5 days, range: 1, 52).

In the buttocks (left and right), bruising peaked at Day 4 in injection types I, III, IV, and V; and peaked at Day 8 in injection type II. In the thigh (left and right), bruising peaked at Day 4 in all injection types. Bruising was substantially reduced at Day 15 in all injection types and regions. The reliability of the outcomes of the bruising analysis (L*A*B* color measurements) was assessed by measuring the change in color coordinates of normal tissue as a comparator. The analysis of normal tissue showed that there was some noise in the bruising measurement, which could be attributed to the technique itself or the environment where the photograph was taken. However, in the treated tissue, the improvement in bruising over time was much greater than the level of noise. Therefore, the bruising analysis can be considered meaningful data. No deaths or treatment-related discontinuations occurred. There were no clinically concerning trends observed in laboratory assessments, vital signs, physical findings, or other observations during the study. There were no clinically concerning trends observed in laboratory assessments, vital signs, physical findings, or other observations during the study.

Considering that injection types II and III were associated with the most safety concerns, these shallow and deep bolus techniques are not recommended in either the buttocks or thighs regardless of efficacy performance. Based on the safety and efficacy data taken together, it is recommended that the buttocks be treated with injection type I (3 shallow injections) or a combination of techniques IV and V (e.g., 4 shallow injections with the fifth injections with the fifth being shallow or deep depending on the dimple depth). The thigh should be treated with injection type IV (a single deep injection plus 4 shallow injections) if the dimple is deep or 5 shallow injections if the dimple is superficial/horizontal.

### Example 2—A Phase 2a, Open-Label Study of CCH in the Treatment of Edematous Fibrosclerotic Panniculopathy of Posterolateral Thighs in Adult Women (Study 212)

Based on the efficacy and safety findings from the Phase 2a and 2b EFP studies, the CCH 0.84 mg dose per treatment area (in two treatment areas; buttocks or thighs 1.68 mg) warranted further investigation in the thighs. Among other objectives, in this study, attempts will be made to visualize the changes in the dimple areas after treatment to evaluate the effect of CCH in tissue and or septae causing the dimple. These data will assessed and compared with histopathology and MRI data to attempt to understand the mechanism of action of CCH. Accordingly, this study is a Phase 2a open-label study of the efficacy and safety of CCH in the treatment of EFP in thighs of adult women.

The expected duration of the study is as follows:
Screening Phase: Up to 28 Days (day −28 to −1) (Duration includes scheduling and conducting MRI Imaging prior to biopsy or dosing)
Titration Phase: N/A
Treatment Phase: 71 Days
Long-term Follow Up: 109 Days (day 72-180) for long-term efficacy assessment to Day 180 (end of study)

All subjects will be screened based on predetermined inclusion and exclusion criteria, e.g., non-pregnant women 18 years of age or older having EFP in the thighs and a body mass index of 20-35 kg/m². Subjects must have a minimum of two (2) well-defined, isolated dimples in each selected treatment area (each thigh) of which 3 will be selected for MRI and biopsy (histopathology) procedure. All subjects will be judged to be in good health based on medical history, a physical examination, and laboratory profiles at screening.

Subjects will receive a treatment course which consists of up to 3 treatment visits (sessions), separated by 21 days (i.e., Days 1, 22 and 43). The volume and dose of injection at each treatment visit will be according to Treatment IV described above. Three treatment visits total 5.04 mg of CCH administered. The CCH formulation is described above.

Selection of dimples to be treated will be at the discretion of the Investigator. Assessments for efficacy will be performed during study visit Days 43, 106, 150 and Day 180. Body-Q assessment will be performed at baseline and Day 71, 120 and Day 180. MRI and histopathology will be performed at the end of Day 71 assessment.

At each treatment visit, the treatment area (selected thigh) of each selected subject will be photographed before and after marking dimples and injection sites while the subject is in a consistent, standardized relaxed standing pose. The Investigator will assess the treated area using an Investigator Global Aesthetic Improvement Scale (I-GAIS) and Hexsel CSS at Days 22, 43, 64, 85, 106, 150 and 180. The subjects' treatment areas will be subjected to Cherry Imaging (www.cherryimaging.com) at visit Day 1, 22, 43, 64, 85, 106, 150 and 180. In addition, the subjects will evaluate the treatment area using Subject Global Aesthetic Improvement Scale (S-GAIS) at Days 22, 43, 64, 85, 106, 150 and 180. The subject assessments will be completed prior to, and independently, of the Investigator assessments at each treatment visit. In addition, the assessments evaluating efficacy will be done before the dimple marking for injection.

The Investigator will conduct live assessments of the treated thigh using the CR-PCSS. Global assessment evaluations will be completed by both the subject (S-GAIS) and the Investigator (I-GAIS). More specifically, the criteria for evaluation are as follows:

1. Efficacy:
a. Investigator Global Aesthetic Improvement Scale (I-GAIS) of the each treated area: 7-level scale ranging from 3 (very much improved) to −3 (very much worse) (measured at Days 43, 106, 150 and 180)
b. Subject Global Aesthetic Improvement Scale (S-GAIS) of each treated area: 7-level scale ranging from 3 (very much improved) to −3 (very much worse) (measured at Days 43, 106 150 and 180)
c. Body-Q will be assessed on Days 106, 150 and 180
d. Hexsel CSS will be assessed at screening and on Days 106, 150 and 180
e. Histopathology (through skin biopsy) will be assessed at baseline, Day 71 and 180 and at baseline, Day 150 and 180.
f. Magnetic Resonance Imaging (MRI) will be assessed on the same subjects for Histopathology at baseline, Day 71 and 180.

2. Safety will be assessed throughout the study through the recording of:
a. Adverse events (AEs) (including those of special interest (AESI); which are adverse events such as bruising, ecchymosis, hematomas, and contusions that occur remote to the site of drug administration, or any hypersensitivity reactions, including anaphylaxis)
b. Vital signs
c. Clinical laboratory tests
d. Immunogenicity assessment (i.e., assessed through the determination of binding and neutralizing anti-AUX-I and anti-AUX-II antibody levels)

The primary endpoint of the study is the proportion of 2-level I-GAIS responders (defined as subjects with +2 or more in the Investigator GAIS assessment) of at least one thigh at Day 180. The primary endpoint of the study is the proportion of 2-level I-GAIS responders (defined as subjects with +2 or more in the Investigator GAIS assessment) of at least one buttock at Day 180.

Secondary endpoints are:
Proportion of subjects at each level of the I-GAIS of treated thigh at Day 22, 43, 64, 85, 106, 150 and 180
Proportion of subjects at each level of the I-GAIS of treated buttock at Day 22, 43, 64, 85, 106, 150 and 180
Proportion of subjects at each level of the S-GAIS of treated thigh at Day 22, 43, 64, 85, 106, 150 and 180
Proportion of subjects at each level of the S-GAIS of treated buttock at Day 22, 43, 64, 85, 106, 150 and 180
Proportion of 1-level I-GAIS responders (defined as subjects with +1 or more in the I-GAIS assessment) of at least one thigh at Days 22, 43, 64, 85, 106, 150 and 180
Proportion of 1-level I-GAIS responders (defined as subjects with +1 or more in the I-GAIS assessment) of at least one buttock at Days 22, 43, 64, 85, 106, 150 and 180
Proportion of 2-level S-GAIS responders (defined as subjects with +2 or more in the S-GAIS assessment) of at least one thigh at Days 22, 43, 64, 85, 106, 150 and 180
Proportion of 2-level S-GAIS responders (defined as subjects with +2 or more in the S-GAIS assessment) of at least one buttock at Days 22, 43, 64, 85, 106, 150 and 180
Proportion of 1-level S-GAIS responders (defined as subjects with +1 or more in the S-GAIS assessment) of at least one thigh at Days 22, 43, 64, 85, 106, 150 and 180
Proportion of 1-level S-GAIS responders (defined as subjects with +1 or more in the S-GAIS assessment) of at least one buttock at Days 22, 43, 64, 85, 106, 150 and 180
Change from baseline in total score of Hexsel CSS to Days 22, 43, 64, 85, 106, 150 and 180 for thigh
Body-Q Appraisal of Cellulite at Day 150 and 180 for thigh

All endpoints will be summarized using appropriate descriptive statistics by time-point or other suitable statistics package. For the histopathology (biopsy) analysis, a pathologist will report on any tissue differences from baseline. For the MRI analysis, images will be acquired and visualized for any tissue changes and any septae morphology differences in pre-treatment and post-treatment.

Applicant expects that this study will establish the efficacy and safety of the CCH according to the endpoints described above.

### Example 3—a Multicenter, Open-Label, Multiple Dose, Phase 3B Study to Assess the Safety and Efficacy of CCH in Adult Women with Mild and Moderate EFP (Study 305)

This is a multicenter, open-label, multiple dose, Phase 3b study to assess the safety and efficacy of CCH in adult women with mild and moderate EFP. Approximately 200 subjects will be screened in order to enroll approximately 150 subjects in 2 cohorts. All subjects will be screened based on predetermined inclusion and exclusion criteria, e.g., non-pregnant women 18-60 years of age having EFP in the thighs and butttocks and a body mass index of 18-30 kg/m². All subjects will be judged to be in good health based on medical history, a physical examination, and laboratory profiles at screening.

Following a screening period of approximately 14 days, qualified subjects will be randomly assigned to 1 of 2 cohorts. Cohort 1 will have approximately 80 subjects each with mild or moderate EFP in the posterolateral thighs; Cohort 2 will have approximately 70 subjects with mild or moderate EFP in the buttocks.

All subjects will receive 0.84 mg of CCH (described above) per treatment area (buttock or thigh) for a total dose of 1.68 mg in both buttocks or both thighs per treatment session during the 3 treatment sessions (Day 1, Day 22 (+7 days), and Day 43 (+14 days). Subjects will return to the site on Days 90 and 180 for endpoints and other study evaluations and follow-up.

For each buttock, a dose of 0.84 mg of CCH in 3.6 mL will be administered as 12 subcutaneous injections (0.3-mL injection administered as three 0.1-mL aliquots per injection) according to Treatment I.

For each thigh, a dose of 0.84 mg of CCH in 18.0 mL will be administered as 12 subcutaneous injections (1.5-mL injection administered as five 0.3-mL aliquots per injection) in each thigh according to Treatment IV, but modified such that the physician can choose an injection depth of ½ inch or 1 inch for the center aliquot. For each thigh, the volume will be 1.5 mL per injection in 5 aliquots of 0.3 mL of CCH and at 0.07 mg/injection for a total of 0.84 mg of CCH in total volume of 18.0 mL. For a two thigh treatment, the total drug will be 1.68 mg in a total volume of 36 mL. Three treatment visits totals to 5.04 mg of CCH. The investigator/physician will be advised to use his/her clinical judgment on the dimple type (based on depth, length, width, etc.) to decide if the central aliquot (aliquot A in FIG. 10) has to be administered deep (1-inch needle at 30 degree angle) or shallow (0.5 inch needle at 30 degree angle). Depending on that decision, the investigator will use either a 1-inch or 0.5 needle to administer the 5-aliquot injection technique on thigh cellulite dimples.

There will be 3 treatment visits of the same dose at intervals of approximately 21-30 days. All completed subjects will have 3 treatment sessions, 21-30 days apart. Total study duration for each subject is approximately 208 days.

The following clinical endpoints will be summarized using appropriate descriptive statistics by time point and cohort:
Change from baseline in I-GAIS at Day 90
Efficacy in buttocks or thighs through the proportion of subjects with improved (+1) or better score on a 7 point I-GAIS scale at Day 90
Efficacy in buttocks or thighs through the proportion of subjects with improved (+1) or better score on a 7 point I-GAIS scale at Day 180
Efficacy in buttocks through mean change from baseline in CR-PCSS at Day 90
Efficacy in buttocks through mean change from baseline in CR-PCSS at Day 180
Patient satisfaction in buttocks or thighs through mean change from baseline in Body Q—Appraisal of Cellulite at Day 90
Patient satisfaction in buttocks or thighs through mean change from baseline in Body Q—Appraisal of Cellulite at Day 180
Proportion of dimples completely/successfully treated in a treatment area.
Mean reduction in total number of dimples from baseline, per treatment visit
Mean dose per cellulite dimple per treatment visit
To assess investigator satisfaction with CCH administration
   Ease of administration (5 pt. Likert Scale)
   Likelihood of incorporating into practice as a treatment for cellulite (5 pt. Likert Scale)
   Overall, rate the ease of use of CCH in treating patients with cellulite?(5 pt. Likert scale)
   Ease of adding on to other aesthetic treatments (other than cellulite) as a same day procedure (5 pt. Likert scale)
To measure dermal thickness of treatment areas via the echogenicity of the dermis (or its individual layers) observed in the ultrasound imaging of the skin
   Mean change in dermal thickness after treatment of cellulite with CCH from baseline to day 90
   Mean change in dermal thickness after treatment of cellulite with CCH from baseline to day 180
To evaluate 3-D images of treatment areas (using Cherry Imaging and 3-D photography)
   Mean volumetric improvement after treatment of cellulite with CCH from baseline to day 90
   Mean volumetric improvement after treatment of cellulite with CCH from baseline to day 180
   Mean change in skin roughness score before and after treatment with CCH at day 90
   Mean change in skin roughness score before and after treatment with CCH at day 180

AEs will be summarized by proportion of subjects reporting each event. Descriptive statistics will be presented for actual and change from baseline at each visit for vital signs and for each clinical laboratory test parameter. Anti-AUX-I and anti-AUX-II antibody levels will be summarized using descriptive statistics for the actual value at the visit.

CR-PCSS (buttock only), Body Q-Appraisal of Cellulite, I-GAIS, 5 point Likert Scale, Cherry Imaging skin roughness scale, Body Dysmorphia Questionnaire (see, e.g., Lifespan.org; bddfoundation.org), and Fitzpatrick skin type will be used for assessment of efficacy. The CR-TCES and PR-TCES scales may also be employed in this example in lieu of or together with the CR-PCSS and PR-PCSS scales.

Applicant expects that this study will establish the efficacy and safety of the CCH according to the endpoints described above.

### Example 4—A Phase 2B, Randomized, Double-Blind, Placebo-Controlled Study of CCH in the Treatment of Edematous Fibrosclerotic Panniculopathy in Thighs (Study 211)

Previous studies have shown improvements in the severity of cellulite in subjects who were subcutaneously treated three times with CCH administered at a dose of 0.84 mg in a single treatment area (e.g., one buttock or one thigh) every 21 days. The majority of adverse events occurred at the site of injection, were mild to moderate in nature, and resolved within several weeks with no recurrence.

In this example, a Phase 2, randomized, double-blind, placebo-controlled study will be conducted of the efficacy and safety of CCH in the treatment of EFP in thighs of adult women. The CCH (1:1 ratio of AUX-I and AUX-II) to be administered is described above. Subjects will be screened for study eligibility within 14 days prior to enrolling in this study. All subjects will be screened based on predetermined inclusion and exclusion criteria, e.g., non-pregnant women 18 years of age or older having EFP in the thighs and a body mass index of 20-35 kg/m². All subjects will be judged to be in good health based on medical history, a physical examination, and laboratory profiles at screening.

Subjects with 2 bilateral left and right posterolateral thighs with moderate or severe level of cellulite as independently assessed by the subject using the PR-TCES scale and by the investigator using the CR-TCES scale and that have thighs with a Hexsel Cellulite Severity Scale (CSS) no greater than 13 will be eligible and a Hexsel CSS depression depth score of 2 or 3. The eligibility of the thighs will be confirmed on Day 1.

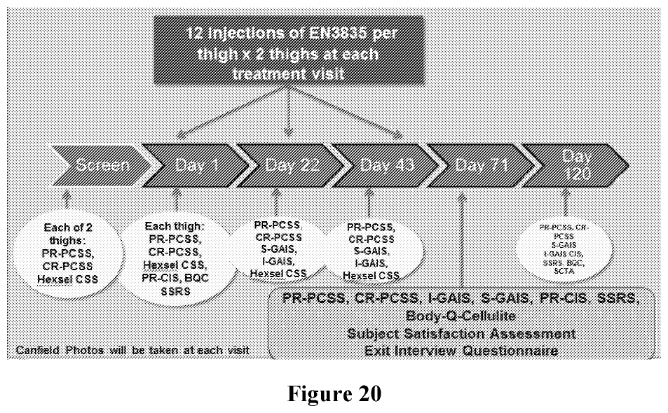

Once the eligibility of the assigned thighs is confirmed, one of the thighs will be randomly assigned as the target thigh; the other thigh will be considered the non-target thigh. The subjects will be randomly assigned to a treatment group (CCH 0.84 mg per thigh or placebo). Both thighs of a subject will be treated with either CCH treatment or placebo treatment. Each subject will receive a treatment course which consists of 3 treatment sessions (i.e., Days 1, 22 and 43). Each treatment session will consist of 12 injections (1.5 mL per injection of CCH 0.07 mg/injection or placebo) in each of the thighs for a total volume of 36 mL (1.68 mg). Selection of dimples to be treated in the assigned thighs will be at the discretion of the Investigator. Primary endpoint analyses will be based on evaluations at Day 71. There will be a follow-up visit at Day 120. Subjects, investigators, site personnel and the sponsor will be blinded to the identification of the target thigh and to the assigned treatment. A flowchart of the study design is illustrated in FIG. 20.

The injection technique that will be used in this study is Treatment IV described above. At each treatment session and at Day 71 and Day 120, each of the thighs will be photographed before and after dimple marking while the subject is in a consistent relaxed standing pose. The subject will assess the digital photographic image (pre-marking) of each assigned thigh using the PR-PCSS to determine the severity of EFP in each of the thighs. The subject will evaluate the thighs using a Subject Global Aesthetic Improvement Scale (S-GAIS), a Patient Reported Cellulite Impact Scale (PR-CIS), Subject Self-Rated Scale (SSRS) a Subject Satisfaction with Cellulite Treatment (SCTA) and the Body Q—Cellulite assessment. Subsequently, the investigator will do live assessments of each of the thighs using the CR-TCES. The subject assessments will be completed prior to and independently of the Investigator assessments at each treatment visit. The investigator will assess each of the treated thighs using an Investigator Global Aesthetic Improvement Scale (I-GAIS). At each visit, all the assessments must be done before the dimple marking. After completion of Day 71 visit assessments and prior to Day 120 visit, a semi-qualitative exit interview on a subset of blinded, randomly selected subjects will be conducted to assess the subjects' evaluation of treatment effect, treatment experience, participation experience, and unintended effects.

Safety will be assessed throughout the study including adverse events of special interest, which include all adverse events such as bruising, ecchymosis, hematomas, and contusions that occur remote to the site of drug administration, or any hypersensitivity reactions, including anaphylaxis, will be recorded and evaluated for seriousness and severity in this study. In addition, local AEs associated with the injection site, including acute (e.g., erythema, bruising, pain, nodules/mass, ulceration, blistering, pruritus, swelling, and/or induration) and/or chronic (e.g., skin thickening, fibrosclerosis, peau d'orange changes) cutaneous adverse events, will be recorded and evaluated for seriousness and severity (as appropriate).

The primary endpoint is the proportion of 2-level composite responders at Day 71 defined as subjects with at least one thigh with:
an improvement in severity from baseline (Day 1 visit) of at least 2 levels of severity in the CR-TCES as assessed live by the Investigator, and
an improvement in severity from baseline of at least 2 levels of severity in the PR-TCES as assessed by the subject while viewing the digital image of the same thigh.

Secondary endpoints are:
The proportion of 2-level composite responders in the target thigh at Day 71 defined as subjects with:
   an improvement in severity from baseline (Day 1 visit) of at least 2 levels of severity in the CR-TCES as assessed live by the Investigator in the target thigh, and
   an improvement in severity from baseline of at least 2 levels of severity in the PR-TCES as assessed by the subject while viewing the digital image of the same thigh.
Proportion of 1-level PR-TCES responders defined as subjects with ≥1-level improvement in PR-TCES severity rating of at least one thigh at Day 71 compared to Day 1
Proportion of 2-level PR-TCES responders defined as subjects with 2-level improvement in PR-TCES severity rating of at least one thigh at Day 71 compared to Day 1
Proportion of 1-level or 2-level S-GAIS responders defined as subjects with ≥1-level improvement (improved, much improved, or very much improved) or ≥2-level improvement (much improved or very much improved) in the S-GAIS assessment of at least one thigh at Day 71
Change from baseline of PR-TCES, CR-TCES, SSRS, PR-CIS total score, Body Q—Cellulite, Hexsel number of dimples, Hexsel depression depth score, Hexsel total score at Day 71

For results, Applicant expects that all of the primary and secondary endpoints will be met, and that the patients will have experienced no unexpected adverse events.

### Example 5—a Phase 1, Open-Label Study to Assess the Safety and Pharmacokinetics of a Single Dose of CCH (3.36 Mg) in Subjects with Edematous Fibrosclerotic Panniculopathy

Previous studies have shown improvements in the severity of cellulite in subjects who were subcutaneously treated three times with CCH administered at a dose of 0.84 mg in a single treatment area (e.g., one buttock or one thigh) every 21 days. The majority of adverse events occurred at the site of injection, were mild to moderate in nature, and resolved within several weeks with no recurrence. Other phase 1 pharmacokinetic (PK) studies involving subcutaneous CCH treatment in one and two treatment areas showed no systemic exposure after treatment. In reality, a physician may desire to administer CCH to more than two treatment areas in a patient during a single session. The CCH safety profile in such a scenario is therefore important.

In this example, a phase 1, open-label PK and safety study was conducted in which subjects were injected with a single 3.36 mg dose, administered as 12 subcutaneous injections in each of the left buttock, right buttock, left posterolateral thigh, and right posterolateral thigh (i.e., 0.84 mg CCH per treatment area).

Following a 21-day screening period, twelve subjects were admitted to the clinical research unit the day before administering CCH (Day −1). The subjects were dosed on Day 1. On Day 2, 24 hours after administration, a PK sample was obtained from each subject, and the subjects were discharged from the clinical research unit. The subjects returned on Days 3, 8, and 22 for outpatient follow-up PK and safety assessments (i.e., 48 hours post-dose, 168 hours post-dose and 504 hours post-dose). All subjects were non-pregnant women 18 years of age or older having EFP in the four treatment areas and a body mass index of 20-35 kg/m². All subjects were judged to be in good health based on medical history, a physical examination, and laboratory profiles at screening.

The administered CCH is described above. Each injection site received a single skin injection of study drug administered as three 0.1 mL aliquots for a total injection volume of 0.3 mL per injection, an injection volume of 3.6 mL per treatment area, and a total injection volume of 14.4 mL. Sixteen syringes (four per treatment area) were prepared for dosing on Day 1. Each syringe contained 0.9 mL of study drug providing for three injections per syringe.

Blood samples were drawn from each subject at the relevant time periods to obtain various PK parameters (e.g., AUC₀₋ₜ, AUC_{0-inf}, Cₘₐₓ, Tₘₐₓ). One subject exhibited comparable pre-dose and post-dose plasma values for AUX-I greater than the limit of quantification (5 ng/mL) at all collection time points, which is consistent with possible interference in the AUX-I ELISAs. In all other subjects, there were no quantifiable levels of AUX-I in the blood plasma at any time point post-dose attributable to the injection of 3.36 mg of the study drug. In addition, there were no quantifiable levels of AUX-II in the blood plasma at any time point post-dose attributable to the injection of 3.36 mg of the study drug. Accordingly, no PK parameters were obtained. This confirms the lack of systemic exposure of CCH following concurrent, subcutaneous administration of 3.36 mg CCH in four treatment areas.

All twelve female subjects received all 48 injections, 12 injections per treatment area, for a total dose of 3.36 mg CCH on Day 1. All subjects experienced at least one treatment-emergent adverse event, none of which led to the discontinuation of treatment or death. There were no serious adverse events. The most frequently reported adverse events were injection site erythema (5 subjects, 41.7%), injection site bruising (2 subjects, 16.7%), injection site nodules (2 subjects, 16.7%). Consequently, no new concerns in the safety profile of CCH were observed with concurrent administration in four treatment areas. The commonly reported events in this study are consistent with the currently known adverse event profile of CCH.

Even though this study was not designed to determine efficacy as an endpoint and the subjects were treated with only one dose of the study drug, CCH demonstrated improvement in EFP based on improvements in the CR-PCSS rating scores, as summarized in Table 28. Negative changes between the CR-PCSS scores at the end of the study and at screening reflects an improvement in cellulite severity. As shown in the table, improvements in cellulite severity were observed in all four treatment areas, as reflected in the negative changes in the means from baseline in all four treatment areas. One subject experienced a two-point reduction in her cellulite severity in both her right buttock and left thigh. Many patients experienced one-point improvements across the four treatment areas. One subject was excluded from the analysis due to missing CR-PCSS scores at the end of the study.

TABLE 28
CR-PCSS Rating and Change from Baseline (Cellulite Severity
Population) in patients administered a single dose of 3.36
mg CCH in four treatment areas (0.84 mg per treatment area)

| | | Left | | Right | | Left | | Right | |
|---|---|---|---|---|---|---|---|---|---|
| | | Buttock | | Buttock | | Thigh | | Thigh | |
| Cellulite Severity | Statistic | (N = 11) | | (N = 11) | | (N = 11) | | (N = 11) | |
| Screening | | | | | | | | | |
| (baseline) | | | | | | | | | |
| None (0) | n (%) | 0 | | 0 | | 0 | | 0 | |
| Almost None (1) | n (%) | 0 | | 0 | | 0 | | 0 | |
| Mild (2) | n (%) | 6 | (54.5) | 4 | (36.4) | 8 | (72.7) | 8 | (72.7) |
| Moderate (3) | n (%) | 4 | (36.4) | 6 | (54.5) | 3 | (27.3) | 3 | (27.3) |
| Severe (4) | n (%) | 1 | (9.1) | 1 | (9.1) | 0 | | 0 | |
| | Mean | 2.5 | (0.69) | 2.7 | (0.65) | 2.3 | (0.47) | 2.3 | (0.47) |
| | (SD) | | | | | | | | |
| Day 22 (end of | | | | | | | | | |
| study) | | | | | | | | | |
| None (0) | n (%) | 0 | | 0 | | 0 | | 0 | |
| Almost None (1) | n (%) | 1 | (9.1) | 2 | (18.2) | 5 | (45.5) | 5 | (45.5) |
| Mild (2) | n (%) | 7 | (63.6) | 6 | (54.5) | 5 | (45.5) | 4 | (36.4) |
| Moderate (3) | n (%) | 3 | (27.3) | 1 | (9.1) | 1 | (9.1) | 2 | (18.2) |
| Severe (4) | n (%) | 0 | | 2 | (18.2) | 0 | | 0 | |
| | Mean | 2.2 | (0.60) | 2.3 | (1.01) | 1.6 | (0.67) | 1.7 | (0.79) |
| | (SD) | | | | | | | | |
| Change from | | | | | | | | | |
| Baseline | | | | | | | | | |
| (Day 22-baseline) | | | | | | | | | |
| −2 | n (%) | 0 | | 1 | (9.1) | 1 | (9.1) | 0 | |
| −1 | n (%) | 5 | (45.5) | 4 | (36.4) | 5 | (45.5) | 7 | (63.6) |
| 0 | n (%) | 5 | (45.5) | 5 | (45.5) | 5 | (45.5) | 3 | (27.3) |
| +1 | n (%) | 1 | (9.1) | 1 | (9.1) | 0 | | 1 | (9.1) |
| +2 | n (%) | 0 | | 0 | | 0 | | 0 | |
| | Mean | −0.4 | (0.67) | −0.5 | (0.82) | −0.6 | (0.67) | −0.5 | (0.69) |
| | (SD) | | | | | | | | |
| Responder Type | | | | | | | | | |
| 2-point Responder | | | | | | | | | |
| Yes | n (%) | 0 | | 1 | (9.1) | 1 | (9.1) | 0 | |
| No | n (%) | 11 | (100) | 10 | (90.9) | 10 | (90.9) | 11 | (100) |
| 1-point Responder | | | | | | | | | |
| Yes | n (%) | 5 | (45.5) | 5 | (45.5) | 6 | (54.5) | 7 | (63.6) |
| No | n (%) | 6 | (54.5) | 6 | (54.5) | 5 | (45.5) | 4 | (36.4) |

The embodiments described herein are intended to be merely exemplary. Persons skilled in the art will understand that variations and modifications may be made without departing from the scope of the invention encompassed by the claims below.

1. A method of reducing the severity of cellulite in a thigh of a human patient, comprising the steps of:
a. providing a collagenase composition having at least two of the following characteristics:
i. Vₘₐₓ (min⁻¹) of about 0.08 to 7.70 (SRC assay), or about 0.3 to 30.5 (GPA assay);
ii. K_{M}, of about 4.1 to 410 nanoMolar (SRC assay), or about 0.03 to 3.1 mM (GPA assay); iii. K_{cat}(sec⁻¹) of about 1.1 to 107 (SRC assay), or about 93 to 9,179 (GPA assay);
iv. 1/K_{cat}, microseconds of about 376 to 37,222 (SRC assay), or about 4 to 428 (GPA assay);
v. K_{cat}/K_{M}, mM⁻¹sec⁻¹ of about 5,140 to 508,814 (SRC assay), or about 60 to 5,934 (GPA assay);
vi. A molecular mass from about 60 kDa to about 130 kDa, or about 70 to about 130 kDa, or about 80 to about 120 kDa, or about 90 to about 120 kDa, or about 100 to about 110 kDa;
vii. A purity by area of at least 80% as measured by reverse phase HPLC (high pressure liquid chromatography);
viii. A potency of about 5,000 to about 30,000 f-SRC units/mg;
ix. A potency of about 175,000 to about 500,000 f-GPA units/mg;
x. A potency of about 5,000 to about 25,000 ABC units/mg;
xi. Less than or equal to 1% by area of an impurity selected from the group consisting of clostripain, gelatinase, and leupeptin;
xii. Less than or equal to 1 cfu/mL bioburden; and
b. injecting a therapeutically effective amount of the collagenase composition according to a treatment regimen selected from the group consisting of Treatments I to V,
wherein an improvement in an appearance of the cellulite is established by a scale or other measurement tools selected from the group consisting of Hexsel Cellulite Severity Scale (Hexsel CSS), Hexsel Depression Depth Score, Likert Scale, Dimple Analysis, Clinician Reported Photonumeric Cellulite Severity Scale (CR-PCSS), Patient Reported Photonumeric Cellulite Severity Scale (PR-PCSS), Investigator Global Aesthetic Improvement Scale (I-GAIS), Subject Global Aesthetic Improvement Scale (S-GAIS), Patient Reported Cellulite Impact Scale (PR-CIS), PR-CIS Abbreviated, Subject Self-Rating Scale (SSRS), Subject Satisfaction with Cellulite Treatment (SSCT), Clinician assessment of cellulite severity (photography or other imagery), Body-Q, Thigh Cellulite Efficacy Scale (PR-TCES; CR-TCES), and a validated photonumeric or other scale used by clinicians and/or patients to assess cellulite severity, improvement, and/or patient satisfaction; or
wherein bruising significantly decreases or resolves in color intensity at between about 3 days and 20 days after a treatment visit.

2. The method of claim 1 wherein the collagenase is injected according to Treatment IV.

3. The method of claim 1 wherein the collagenase composition comprises AUX-I and AUX-II having the following characteristics:
a. AUX-I (SRC assay):
i. Vmax, min⁻¹: About 0.08 to 7.70
ii. K_{M}: About 4.1 to 410 nanoMolar
iii. K_{cat}, sec⁻¹: About 1.1 to 107
iv. 1/K_{cat}, microseconds: About 376 to 37,222
v. K_{cat}/K_{M}, mM⁻¹sec⁻¹: About 5,140 to 508,814
b. AUX-II (GPA assay)
i. Vₘₐₓ, min⁻¹: About 0.3 to 30.5
ii. K_{M}, mM: About 0.03 to 3.1
iii. K_{cat}, sec⁻¹: About 93 to 9,179
iv. 1/K_{cat}, microseconds: About 4 to 428
v. K_{cat}/K_{M}, mM⁻¹sec⁻¹: About 60 to 5,934.

4. (canceled)

5. The method of claim 1 wherein the composition comprises at least 3 of the characteristics.

6. The method of claim 1 wherein the composition comprises at least 4 of the characteristics.

7. The method of claim 1 wherein the composition comprises at least 5 of the characteristics.

8. The method of claim 1 wherein the composition comprises about 1 mg to 20 mg of one or more collagenases.

9. (canceled)

10. (canceled)

11. The method of claim 1 wherein the composition has a potency of about 15,000 ABC units/mg to 20,000 ABC units/mg and the therapeutically effective amount is about 1 mg to 20 mg.

12. The method of claim 1 wherein the therapeutically effective amount is about 1 mg to 10 mg and the composition has a potency of about 20,000 to about 30,000 f-SRC units/mg or about 175,000 to about 300,000 f-GPA units/mg.

13. The method of claim 1 wherein when the treatment is administered to a population of patients, the treatment results in at least 5% of patients maintaining their level of improvement versus pretreatment baseline for at least 71 days after the initial dose.

14. The method of claim 13 wherein at least 10% of patients maintaining their level of improvement versus pretreatment baseline for at least 71 days after the initial dose.

15. The method of claim 13 wherein at least 20% of patients maintaining their level of improvement versus pretreatment baseline for at least 71 days after the initial dose.

16. The method of claim 1 wherein when the treatment is administered to a population of patients, the treatment results in at least 5% of patients demonstrating improvement versus pretreatment baseline and showing an additional increase in improvement over time.

17. The method of claim 1 wherein the treatment results in at least one of the following efficacy endpoints as measured by CR-PCSS and/or PR-PCSS:
a. An improvement in severity at Day 22, 43, 71, 90, 180 or 365 from baseline (pretreatment “Day 1”) of at least 2 levels of severity in the CR-PCSS as assessed live by the clinician of the target thigh;
b. An improvement in severity at Day 22, 43, 71, 90, 180 or 365 from baseline (Day 1) of at least 2 levels of severity in the PR-PCSS as assessed by the patient while viewing the digital image of the target thigh;
c. An improvement demonstrated by a 2-level composite response at Day 22, 43, 71, 90, 180 or 365 defined as a patient with an improvement from baseline of at least 2 levels of severity in the CR-PCSS and an improvement from baseline of at least 2 levels of severity in the PR-PCSS;
d. An improvement in severity at Day 22, 43, 71, 90, 180 or 365 from baseline (Day 1) of at least 1 level of severity in the CR-PCSS as assessed live by the clinician of the target thigh;
e. An improvement in severity at Day 22, 43, 71, 90, 180 or 365 from baseline (Day 1) of at least 1 level of severity in the PR-PCSS as assessed by the patient while viewing the digital image of the target thigh;
f. An improvement demonstrated by a 1-level composite response at Day 22, 43, 71, 90, 180 or 365 defined as a patient with an improvement from baseline of at least 1 level of severity in the CR-PCSS and an improvement from baseline of at least 1 level of severity in the PR-PCSS; and
g. In a population of patients who all had CR-PCSS ratings of moderate or severe, the improvement in at least one treatment area was statistically significant compared to placebo wherein the improvement is one or more of a. to f. above.

18. The method of claim 1 wherein the treatment results in at least one of the following efficacy endpoints as measured by Hexsel Depression Depth Score:
a. An improvement in severity at Day 22, 43, 71, 90, 180 or 365 from baseline (pretreatment “Day 1”) of at least 2 levels of severity in the Hexsel Depression Depth Score as assessed live by the clinician of the target thigh;
b. An improvement in severity at Day 22, 43, 71, 90, 180 or 365 from baseline (Day 1) of at least 2 levels of severity in the Hexsel Depression Depth Score as assessed by the patient while viewing the digital image of the target thigh;
c. An improvement demonstrated by a 2-level composite response at Day 22, 43, 71, 90, 180 or 365 defined as a patient with an improvement from baseline of at least 2 levels of severity in the Hexsel Depression Depth Score by clinician assessment and an improvement from baseline of at least 2 levels of severity in the Hexsel Depression Depth Score by patient assessment;
d. An improvement in severity at Day 22, 43, 71, 90, 180 or 365 from baseline (Day 1) of at least 1 level of severity in the Hexsel Depression Depth Score as assessed live by the clinician of the target thigh;
e. An improvement in severity at Day 22, 43, 71, 90, 180 or 365 from baseline (Day 1) of at least 1 level of severity in the Hexsel Depression Depth Score as assessed by the patient while viewing the digital image of the target thigh;
f. An improvement demonstrated by a 1-level composite response at Day 22, 43, 71, 90, 180 or 365 defined as a patient with an improvement from baseline of at least 1 level of severity in the Hexsel Depression Depth Score by clinician assessment and an improvement from baseline of at least 1 level of severity in the Hexsel Depression Depth Score by patient assessment; and
g. In a population of patients who all had Hexsel CSS ratings of medium or deep depressions, the improvement in at least one treatment area was statistically significant compared to placebo wherein the improvement is one or more of a. to f. above.

19. The method of claim 1 wherein the treatment results in at least one of the following efficacy endpoints as measured by dimple analysis wherein:
a. depth decreases by at least 5%;
b. width decreases by at least 5%;
c. length decreases by at least 5%;
d. overall volume decreases by at least 5%; and
e. surface area decreases by at least 5%.

20.-51. (canceled)

52. The method of claim 1 wherein the treatment results in at least one of the following efficacy endpoints as measured by CR-TCES and/or PR-TCES:
a. An improvement in severity at Day 22, 43, 71, 90, 180 or 365 days from baseline (pretreatment “Day 1”) of at least 2 levels of severity in the CR-TCES as assessed live by the clinician of the target thigh;
b. An improvement in severity at Day 22, 43, 71, 90, 180 or 365 from baseline (Day 1) of at least 2 levels of severity in the PR-TCES as assessed by the patient while viewing the digital image of the target thigh;
c. An improvement demonstrated by a 2-level composite response at Day 22, 43, 71, 90, 180 or 365 defined as a patient with an improvement from baseline of at least 2 levels of severity in the CR-TCES and an improvement from baseline of at least 2 levels of severity in the PR-TCES;
d. An improvement in severity at Day 22, 43, 71, 90, 180 or 365 from baseline (Day 1) of at least 1 level of severity in the CR-TCES as assessed live by the clinician of the target thigh;
e. An improvement in severity at Day 22, 43, 71, 90, 180 or 365 from baseline (Day 1) of at least 1 level of severity in the PR-TCES as assessed by the patient while viewing the digital image of the target thigh;
f. An improvement demonstrated by a 1-level composite response at Day 22, 43, 71, 90, 180 or 365 defined as a patient with an improvement from baseline of at least 1 level of severity in the CR-TCES and an improvement from baseline of at least 1 level of severity in the PR-TCES; or
g. In a population of patients who all had CR-TCES ratings of moderate or severe, the improvement in at least one treatment area was statistically significant compared to placebo wherein the improvement is one or more of a. to f. above.
